(19) United States
(12) Patent Application Publication
(10) Pub. No.: US 2024/0301417 A1
(43) Pub. Date: Sep. 12, 2024

(54) METHODS AND COMPOSITIONS FOR TREATING CHRONIC LIVER DISEASE AND HEPATOCELLULAR CARCINOMA

(71) Applicant: Sanford Burnham Prebys Medical Discovery Institute, La Jolla, CA (US)

(72) Inventors: Cynthia LEBEAUPIN, San Diego, CA (US); Randal J. KAUFMAN, San Diego, CA (US); Clementine DRUELLE, San Diego, CA (US)

(21) Appl. No.: 18/570,892

(22) PCT Filed: Jun. 15, 2022
(86) PCT No.: PCT/US2022/033647

§ 371 Date: Dec. 15, 2023

Related U.S. Application Data
(60) Provisional application No. 63/211,283, filed on Jun. 16, 2021.

(51) Int. Cl. C12N 15/113; A61K 31/421; A61K 31/427; A61K 31/433; A61K 31/4439; A61K 31/444; A61K 31/4545; A61K 31/497; A61K 31/506; A61K 31/5377; A61K 49/00; A61P 35/00

(52) U.S. Cl. CPC C12N 15/113; A61K 31/421; A61K 31/427; A61K 31/433; A61K 31/4439; A61K 31/444; A61K 31/4545; A61K 31/497; A61K 31/506; A61K 31/5377; A61K 49/0008; A61P 35/00; C12N 2310/11; C12N 2310/351

(57) ABSTRACT

Described herein are compositions and methods useful for detecting and treating pathologies associated with ATF in hepatocytes. A method of detecting a condition, a method of inhibiting a protein, a method of inhibiting a gene, and a method of treatment are disclosed herein. The present disclosure includes, but is not limited to, the development and testing of Atf6-specific short, synthetic, single-stranded antisense oligodeoxynucleotides (ASOs) in vitro and in vivo and future screening for ATF6 inhibitors.

### CROSS-REFERENCE

This patent application claims the benefit of U.S. Provisional Patent Application 63/211,283, filed on Jun. 16, 2021, which is hereby incorporated by reference in its entirety.

### BACKGROUND

Fatty liver disease is driven by several causes, including hypernutrition, excessive alcohol consumption and toxicant exposure, all of which can trigger steatosis (or the accumulation of lipids within the liver). A quarter of the world's population suffers from NAFLD that can progress to steatohepatitis (NASH), characterized by inflammation, cell injury and death as well as fibrosis. This chronic and progressive disease risks evolving to end-stage liver disease like cirrhosis and even hepatocellular carcinoma (or HCC), the primary form of liver cancer and an aggressive malignancy. HCC is the 4th leading cause of cancer death worldwide. The emergence of anti-viral therapies and the exponential rise in obesity has led to projections that liver cancer arising from NAFLD/NASH will outnumber cancer arising from hepatitis. The more this disease progresses, the less reversible it becomes, making it urgent to understand the mechanisms involved. Presently, the mainstay of treatment for NAFLD and its progression is lifestyle changes (diet and exercise) or surgery (like gastric bypass in these early stages, or liver transplantation in advanced stages of scarring and damage of the liver). No approved curative treatment exists, and no reliable biomarkers are yet available. Thus, there remains a need to elucidate the role played by the stress-responsive UPR transducer ATF6 in HCC.

### SUMMARY

Disclosed herein, in some embodiments, are methods for preventing and/or treating metabolic diseases and liver cancer by modulating the expression or function of ATF6 in the liver.

ATF6 regulates an adaptive response. Examples disclosed herein demonstrate a novel discovery that ATF6 promotes a detrimental response leading to NASH/HCC and show that ATF6 is sufficient and necessary to drive HCC. Inhibition of ATF6 signaling is a novel therapeutic strategy. Given the processing and activation steps of ATF6 (detailed below), there are many ways to target ATF6. This includes, but is not limited to, sequestering ATF6 as a transmembrane protein in the ER, preventing trafficking of full-length ATF6 from the ER to the Golgi, preventing ATF6 processing by serine proteases in the Golgi, and/or preventing trafficking of the soluble and active ATF6p50 fragment to the nucleus for transcriptional activation of target genes.

The present disclosure disclosed herein provides methods for identifying agents that inhibit ATF6 in hepatocytes as a therapeutic strategy in chronic liver disease and liver cancer. This includes, but is not limited to, the development and testing of Atf6-specific short, synthetic, single-stranded oligodeoxynucleotides (ASOs) in vitro and in vivo and future screening for ATF6 inhibitors.

In an embodiment, a method of treating a hepatic condition comprises administering a pharmaceutical composition comprising a therapeutically effective amount of a synthetic activating transcription factor inhibitor (ATFi), wherein the synthetic ATFi modulates signaling of an activating transcription factor (ATF) in a cell via an interference with a cellular pathway.

In some embodiments the interference with the cellular pathway includes sequestering an ATF protein as a transmembrane protein in the endoplasmic reticulum, preventing trafficking of the ATF protein from the endoplasmic reticulum to the Golgi apparatus, cleaving the ATF protein using proteases, blocking trafficking of a soluble and active ATF fragment to a nucleus of the cell, or a combination thereof. In some embodiments the synthetic ATFi is a synthetic antisense oligonucleotide. In some embodiments, the synthetic antisense oligonucleotide knocks down an endonuclease transcript and reduces levels of a targeted protein via binding to a target mRNA. In some embodiments, the synthetic antisense oligonucleotide is conjugated to a triantennary N-acetyl galactosamine (GalNAc). In some embodiments, the GalNAc targets asialoglycoprotein receptors. In some embodiments, the hepatic condition includes non-alcoholic fatty liver disease (NAFLD), non-alcoholic steatohepatitis (NASH), hepatocyte ballooning, hepatocyte cell death and fibrosis, cirrhosis and hepatocellular carcinoma. In some embodiments, the pharmaceutical composition is administered to the subject via subcutaneous injection, intramuscular injection, or intravenous injection. In some embodiments, the ATF is ATF6.

In an embodiment, a method of detecting a hepatic condition in a subject comprises administering a therapeutically effective amount of a pharmaceutical composition to the subject having the hepatic condition, wherein the pharmaceutical composition comprises an activating transcription factor inhibitor (ATFi) configured to modulate an activating transcription factor (ATF), assessing an efficacy of the ATFi via comparing the levels of the ATF before administering the ATFi to the subject and the levels of the ATF after administering the ATFi to the subject, and measuring a biomarker level associated with the hepatic condition after administering the ATFi to the subject, thereby detecting the hepatic condition.

In some embodiments, the biomarker is screened via digital spatial profiling. In some embodiments, the ATFi is an antisense oligonucleotide. In some embodiments, the ATFi is a small-molecule inhibitor. In some embodiments, assessing the efficacy of the ATFi is performed in vitro. In some embodiments, assessing the efficacy of the ATFi is performed in vivo. In some embodiments, the hepatic condition includes non-alcoholic fatty liver disease (NAFLD), non-alcoholic steatohepatitis (NASH), hepatocyte ballooning, hepatocyte cell death and fibrosis, cirrhosis and hepatocellular carcinoma. In some embodiments, the pharmaceutical composition is administered to the subject via subcutaneous injection, intramuscular injection, or intravenous injection. In some embodiments, the ATF is ATF6.

In an embodiment, a method of inhibiting expression of an activating transcription factor (ATF) gene in a cell comprises administering to a subject a therapeutically effective amount of a pharmaceutical composition comprising a synthetic activating transcription factor inhibitor (ATFi) and a suitable delivery vehicle.

In some embodiments, the synthetic ATFi is a synthetic antisense oligonucleotide. In some embodiments, the synthetic antisense oligonucleotide knocks down a gene encoding an ATF via binding to a target mRNA. In some embodiments, the ATF is ATF6. In some embodiments, the synthetic antisense oligonucleotide is conjugated to a triantennary N-acetyl galactosamine (GalNAc). In some embodiments, the GalNAc targets asialoglycoprotein receptors. In some embodiments, the cell is a hepatocyte. In some embodiments, the hepatic condition includes non-alcoholic fatty liver disease (NAFLD), non-alcoholic steatohepatitis (NASH), hepatocyte ballooning, hepatocyte cell death and fibrosis, cirrhosis and hepatocellular carcinoma. In some embodiments, the pharmaceutical composition is administered to the subject via subcutaneous injection, intramuscular injection, or intravenous injection. In some embodiments, the suitable delivery vehicle is selected from liposomes, nanoparticles, microparticles, microspheres, lipid particles, vesicles, polyaxamer, and polycationic material.

In an embodiment, a method of inhibiting an activating transcription factor in a cell comprises administering to a subject a therapeutically effective amount of a pharmaceutical composition comprising a synthetic activating transcription factor inhibitor (ATFi) and a suitable delivery vehicle, and generating a cellular response, wherein the synthetic ATFi, following administration to the subject, couples to a receptor in the cell, thereby inhibiting the ATF in the cell.

In some embodiments, the synthetic ATFi is a synthetic antisense oligonucleotide. In some embodiments, wherein the synthetic antisense oligonucleotide knocks down an endonuclease transcript and reduces levels of a targeted protein via binding to a target mRNA. In some embodiments, the targeted protein is an ATF6 protein. In some embodiments, the synthetic antisense oligonucleotide is conjugated to a triantennary N-acetyl galactosamine (GalNAc). In some embodiments, the GalNAc targets asialoglycoprotein receptors. In some embodiments, the cell is a hepatocyte. In some embodiments, the subject has a hepatic condition. In some embodiments, the hepatic condition includes non-alcoholic fatty liver disease (NAFLD), non-alcoholic steatohepatitis (NASH), hepatocyte ballooning, hepatocyte cell death and fibrosis, cirrhosis and hepatocellular carcinoma. In some embodiments, the pharmaceutical composition is administered to the subject via subcutaneous injection, intramuscular injection, or intravenous injection. In some embodiments, the suitable delivery vehicle selected from liposomes, nanoparticles, microparticles, microspheres, lipid particles, vesicles, polyaxamer, and polycationic material. In some embodiments, the subject is a human. In some embodiments, the subject can be over 30, 40, 50, 60 70 or 80 years of age, or an age within a range defined by any of the preceding numbers. In some embodiments, the ATFi can be a small molecule inhibitor. In some embodiments, the small molecule inhibitor can be Ceapin A7, or a pharmaceutically acceptable salt thereof. In some embodiments, the small molecule inhibitor comprises a compound of Table 1, Table 2, or Table 3, as disclosed herein, or a pharmaceutically acceptable salt thereof. In some embodiments, the compound of Table 1, Table 2, or Table 3, or a pharmaceutically acceptable salt thereof, can have at least one hydrogen atom with an isotopic distribution in proportional amounts different to those usually found in nature at any position that a hydrogen atom is present. In some embodiments, the compound of Table 1, Table 2, or Table 3, or a pharmaceutically acceptable salt thereof, can have one, two, three, four, five or six hydrogen atoms with deuterium in proportional amounts greater than usually found in nature. In some embodiments, each of the one, two, three, four, five or six hydrogen atoms independently includes at least 52.5% deuterium incorporation, at least 60% deuterium incorporation, at least 67.5% deuterium incorporation, at least 75% deuterium incorporation, at least 82.5% deuterium incorporation, at least 90% deuterium incorporation, at least 95% deuterium incorporation, at least 97% deuterium incorporation, at least 99% deuterium incorporation, or at least 99.5% deuterium incorporation. In some embodiments, each of the one, two, three, four, five or six hydrogen atoms includes less than 99.9% deuterium incorporation.

### INCORPORATION BY REFERENCE

All publications, patents, and patent applications mentioned in this specification are incorporated by reference herein to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the present disclosure are set forth with particularity in the appended claims. A better understanding of the features and advantages of the present present disclosure will be obtained by reference to the following detailed description that sets forth illustrative embodiments, in which the principles of the present disclosure are utilized, and the accompanying drawings of which:

FIG. 1, Panels A-E depicts that 2 week hepatocyte-specific TG-ATF6p50 induction causes hepatomegaly and accumulation of lipids/cholesterol. Panel A depicts a targeting strategy for the transgenic ATF6p50 mice. Cre-mediated recombination is achieved by breeding floxed mice to Cre-specific mice or injecting AAV-Cre and results in expression of the transgenic ATF6p50-HA-tagged allele because of Cre-mediated removal of the STOP cassette. Panel B depicts the experimental protocol as disclosed herein. Transgenic-ATF6p50fl/+ mice aged 8-10 weeks old were injected with AAV8-TBG-eGFP for the wild-type (WT) control group or AAV8-TBG-iCRE for the Transgenic-ATF6p50Hep experimental group (n=7 per group). Mice were intraperitoneally injected with 100% D20 at a volume of 35 μl/g mouse 16 days following GFP or CRE injection. 8% D20 was administered via the drinking water until sacrifice. 6 h prior to sacrifice, serum and liver tissue harvest, mice were fasted for 6h. Panel C depicts that compared to WT mice, Transgenic-ATF6p50Hep mice have greater liver/body weight ratio and thus present signs of hepatomegaly. Panel D depicts that oil red 0 staining revealed stronger neutral lipid accumulation within the liver in Transgenic-ATF6p50Hep mice compared to WT mice. Panel E depicts that heavy water labeling and mass spectrometry revealed a 20% increase in free cholesterol in Transgenic-ATF6p50Hep mice compared to WT mice.

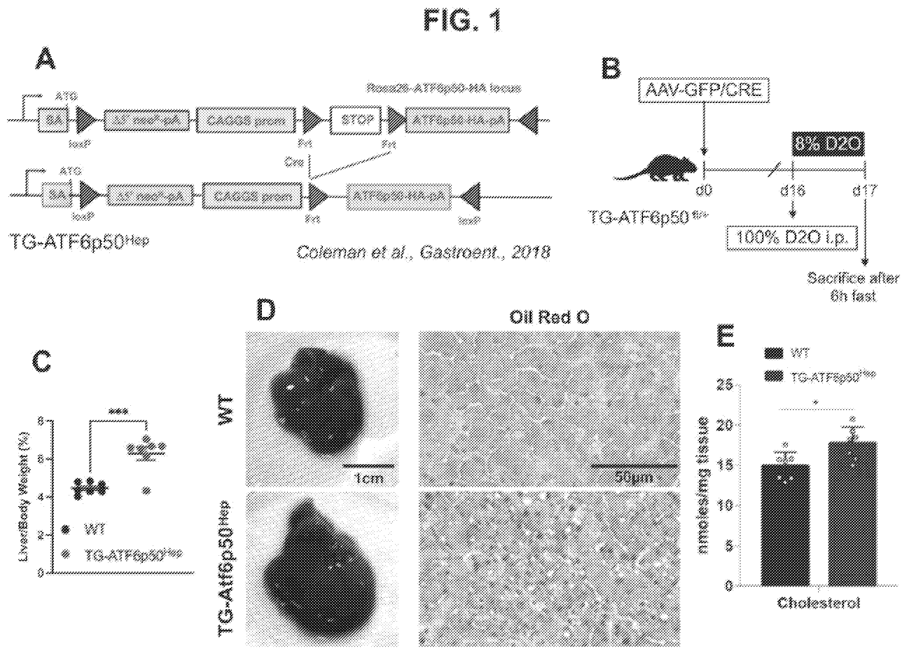

FIG. 2, Panels A-C depicts that 2 week hepatocyte-specific TG-ATF6p50 induction promotes cholesterol synthesis and upregulation of NRF2-induced genes. Panel A depicts western blotting from liver tissue revealed that 2 wk-hepatocyte-specific transgenic-ATF6p50 induction promotes HMGCS1, the first rate-limiting enzyme in cholesterol biogenesis, and STARD1, a mitochondrial cholesterol transporter and a rate-limiting step in bile acid synthesis. There was a down-regulation of fructose 1,6 bisphosphatase (FBP1). Panel B depicts that RT-qPCR from liver tissue revealed similar results at the mRNA level, in addition to a significant increase in NRF2-induced gene expression, notably detoxification and antioxidant transcripts (Cbr3, Gstm1, Gsta1/2, Nqo1, Ho-1). Panel C depicts that ATF6 activation in the liver, and potential FBP1/NRF2 and STARD1 interactions, drive cholesterol synthesis that may be detrimental in the liver, potentially in HCC. Using RNAseq and CHIP-seq data, interactions that link ER stress with cholesterol metabolism to drive HCC progression are identified.

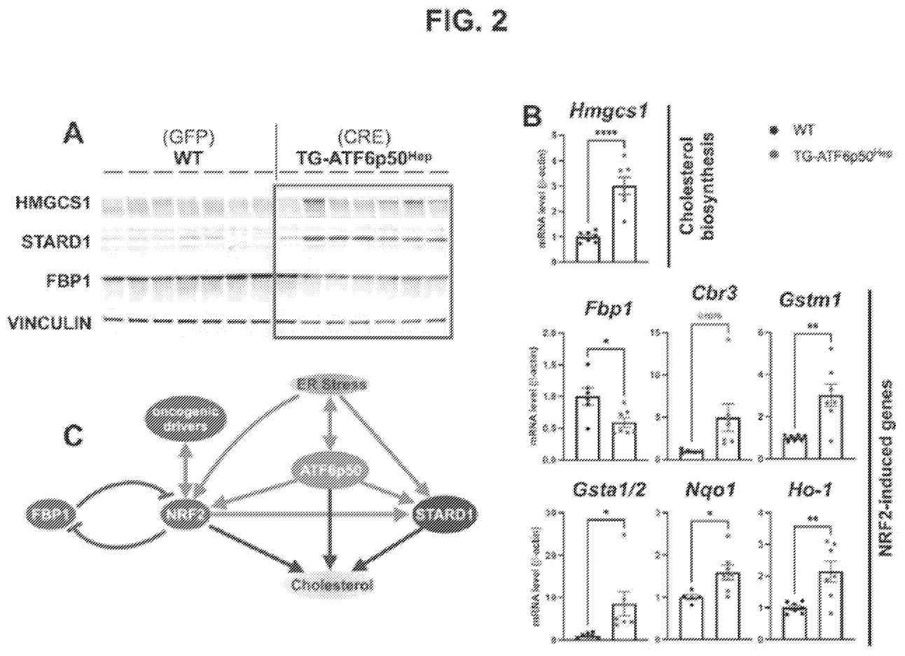

FIG. 3, Panels A-B depict that 2 week hepatocyte-specific TG-ATF6p50 induction activates UPR chaperones (BiP, GRP94) and CHOP. Panel A depicts western blotting from liver tissue revealed that transgenic ATF6p50 induction significantly activates the ER chaperones GRP94 and BiP as well as CHOP. GFP is the positive control for the GFP-injected transgenic floxed mice (WT group), and HA is the second positive control, in addition to the strong p50 activation, for the CRE-injected transgenic floxed mice (Transgenic-ATF6p50Hep group). Panel B depicts RT-qPCR from liver tissue revealed similar results at the mRNA level.

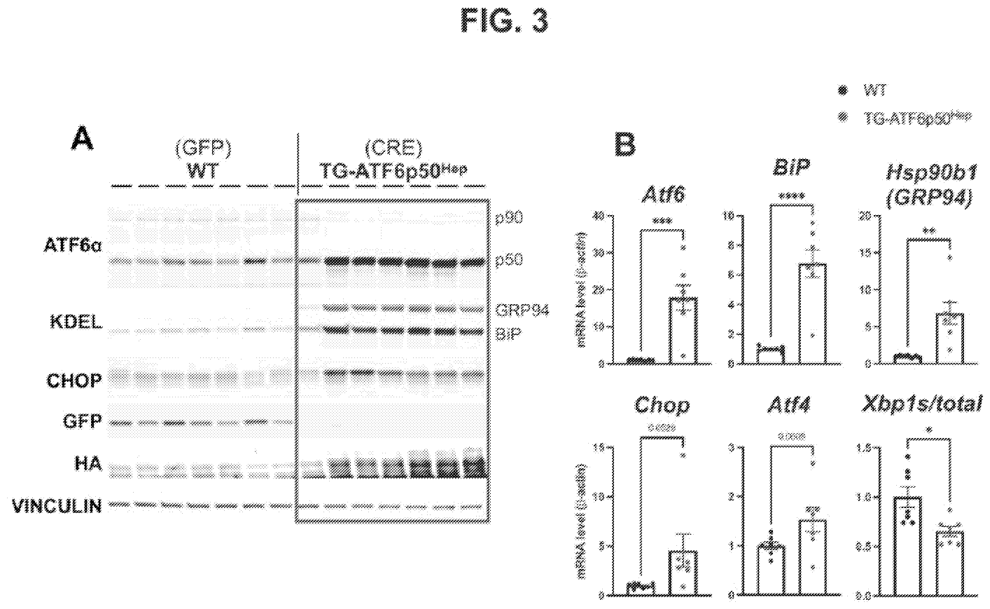

FIG. 4, Panels A-E depicts that TG-ATF6p50 drives DEN+HFD-induced liver cancer. Panel A depicts the experimental protocol disclosed herein. Transgenic-ATF6p50^{fl/+} mice were intraperitoneally injected with DEN (25 mg/kg) at 2 wks old, and subsequently injected with AAV8-TBG-eGFP (n=6 male, n=5 female) or AAV8-TBG-iCRE (n=7 male, n=5 female) at the start of high-fat diet (HFD) feeding at 6 wks old. Mice were sacrificed after 24 wks of diet. Serum and liver tissue were harvested. Additional data (not shown) indicates that TG-ATF6p50 also spontaneously develop HCC within lyr. Panel B depicts quantification of liver/body weight (top) and surface tumor number (bottom) showing an increase in both parameters in Transgenic-ATF6p50^{Hep} mice compared to WT mice. Panel C depicts representative images of harvested liver tissue. Panel D depicts representative H&E staining of liver tissue with tumor nodules encircled. Panel E depicts representative HA-positive staining of harvested liver tissue from Transgenic-ATF6p50Hep mice showing only nuclear localization within tumors compared to the surrounding tissue, which displays both cytoplasmic and nuclear HA-positivity. This may indicate activation of ATF6-dependent transcriptional signaling within HCC tissue.

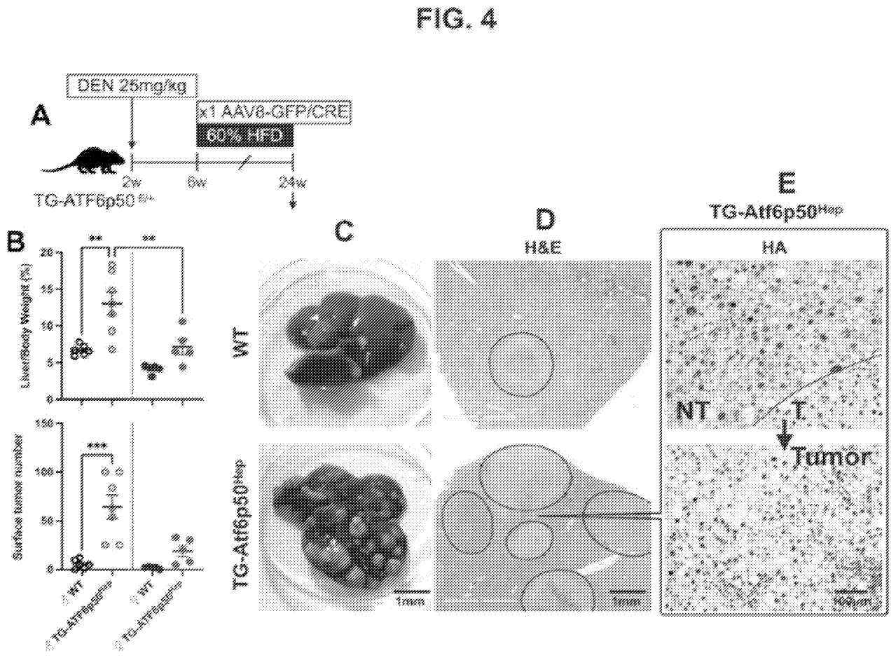

FIG. 5 depicts that TG-ATF6p50 drives DEN+HFD-induced liver injury, bile acid and cholesterol accumulation in the serum. Serum levels of Alanine Aminotransferase (ALT; left), Bile Acids (BA; middle) and Cholesterol were determined using VetScan Mammalian Liver Profile reagent rotors with the VetScan Chemistry Analyzer. Transgenic-ATF6p50Hep mice had consistently elevated ALT levels, indicative of hepatocyte injury, bile acids and cholesterol compared to WT mice over the course of the HFD.

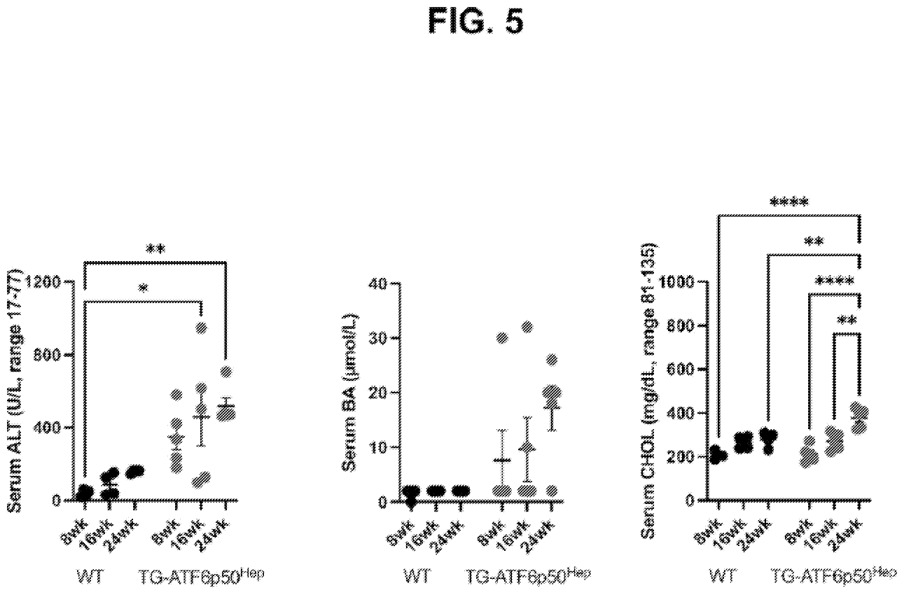

FIG. 6, Panels A-C depicts that TG-ATF6p50 drives DEN+HFD-induced UPR activation and FBP1 downregulation promoting HCC. Western blotting (Panel A) and immunohistochemistry (Panel B) from liver tissue revealed an accumulation of BiP and GRP94 protein, and an increase in mRNA levels (Panel C), in Transgenic-ATF6p50Hep mice, especially in tumor tissue, compared to WT. Although CHOP levels are higher in transgenic nontumoral tissue, they are significantly reduced in tumor tissue of Transgenic-ATF6p50Hep mice.

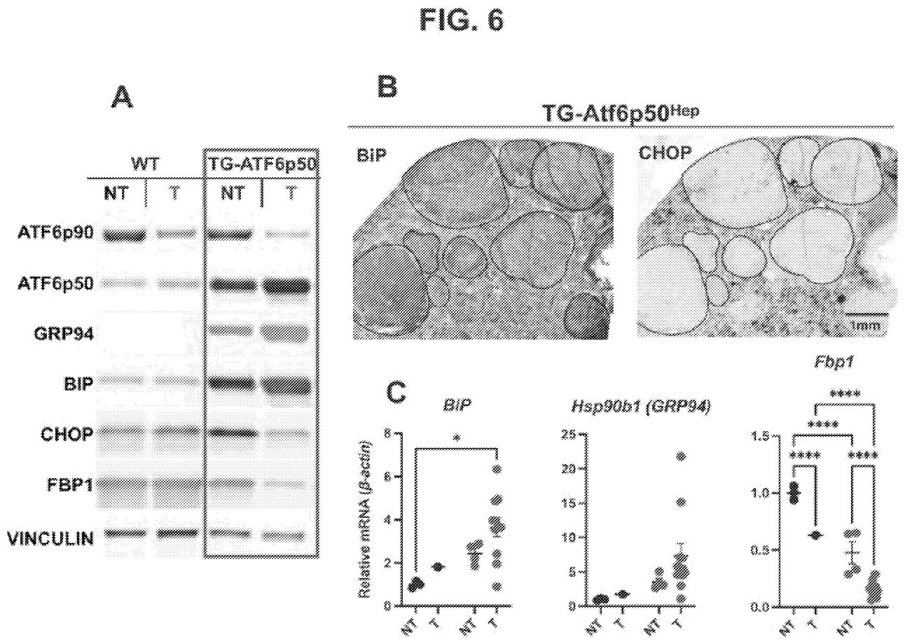

FIG. 7, Panels A-D depicts that full-body Atf6 deletion attenuates HCC development in DEN+HFD model. Panel A depicts the experimental protocol. Atf6+/+ (WT, n=9) and Atf6−/− (KO, n=8) mice were intraperitoneally injected with DEN (25 mg/kg) at 2 wks old, and subsequently fed a high-fat diet HFD starting at 6 wks old. Mice were sacrificed after 32 wks of diet. Serum and liver tissue were harvested. Additional data (not shown) presenting similar findings that Atf6 deletion attenuates HCC development when these mice are crossed with MUP-uPA mice and after long-term western diet feeding in liver-specific Atf6-deleted mice. When crossed with MUP-uPA mice and fed a HFD, full-body Atf6 deletion protects against HCC. Atf6^{ΔHep} livers also do not exhibit liver damage, steatosis or tumors after Western diet feeding. Panel B depicts quantification of liver/body weight (top) and surface tumor number (bottom) showing a reduction in both parameters in Atf6−/− mice compared to Atf6+/+ mice. Panel C depicts representative images of harvested liver tissue. Panel D depicts representative H&E staining of liver tissue with tumor nodules encircled and enlarged.

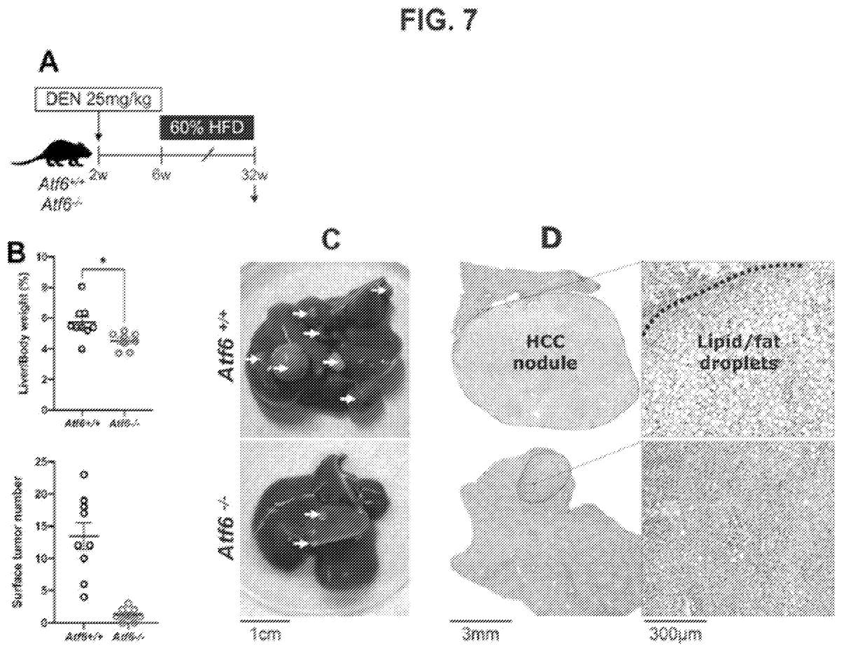

FIG. 8, Panels A-B depicts full-body Atf6 deletion reduces lipid accumulation and ER stress DEN-HFD model. Panel A depicts that quantification (left) and representative transmission electron microscopy images (right) revealed less accumulation of lipid droplets within the tumor tissue of Atf6−/− mice compared to Atf6+/+ mice. Panel B depicts that quantification (left) and representative transmission electron microscopy images (right) revealed less ER stress in tumor tissue of Atf6−/− mice, evident when comparing the dilated appearance of the ER in the Atf6+/+ tumor tissue.

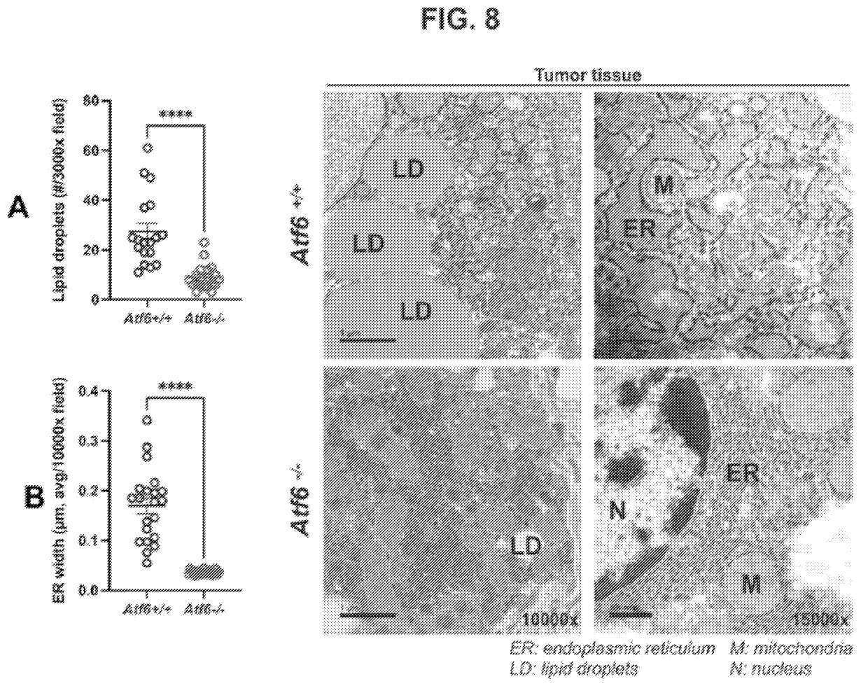

FIG. 9, Panels A-B depict evidence of Atf6-dependent induction of ER stress proteins in tumoral, but not non-tumoral liver tissue. Panel A depicts that western blotting from liver tissue revealed evidence of ER stress protein accumulation in tumor vs. nontumor tissue in Atf6+/+ mice. Panel B depicts representative immunohistochemistry of Atf6++ tumor tissue stained with alpha fetoprotein (AFP, left) and BiP (right).

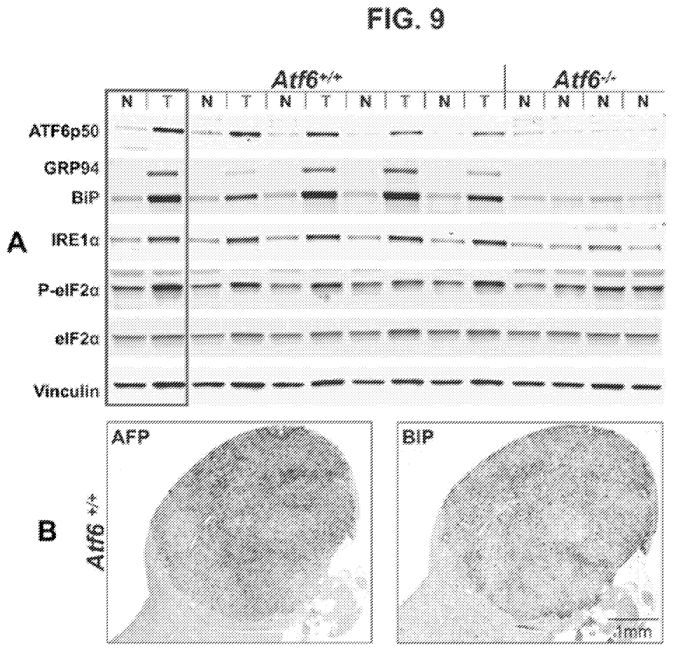

FIG. 10, Panels A-D depicts that hepatocyte-specific Atf6 deletion protects from oncogene-induced HCC. Panel A depicts the experimental protocol. Atf6f/f; AlbCre− (WT, n=8 male, n=4 female) and Atf6f/f; AlbCre+ (KO, n=6 male, n=6 female) mice were hydrodynamically injected with mutant K-RAS and sh-p53 with sleeping beauty transposase for oncogene-induced HCC. Mice were sacrificed and liver tissue was harvested after 30 days. Panel B depicts the survival curve showing improved survival of Atf6−/− vs. Atf6+/+ mice after oncogene injection. Panel C depicts representative images of harvested liver tissue. Panel D depicts that hepatocyte-specific Atf6-deleted mice developed less tumors than WT mice, evident by reduced tumor #per liver and tumor volume, in both males (left) and females (right), showing that Atf6 deletion is truly protective.

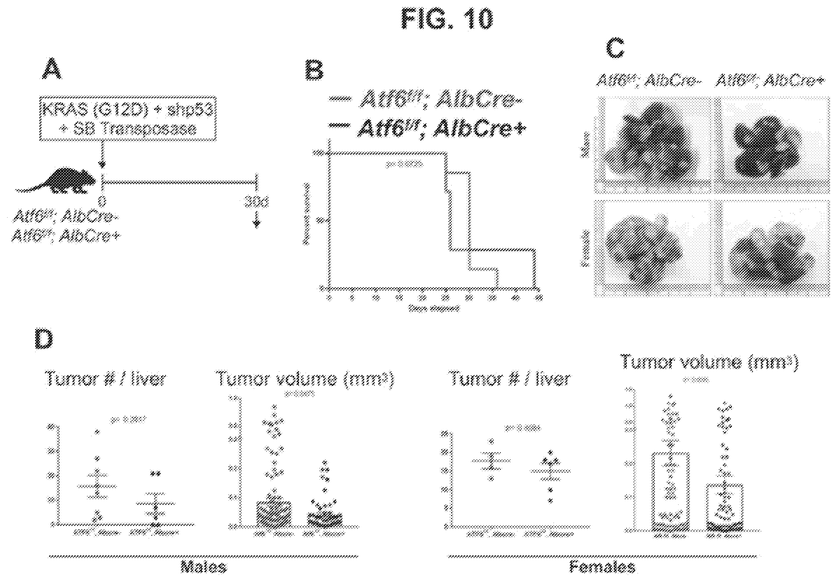

FIG. 11, Panels A-C depicts evidence for ATF6p50 activation in human NAFLD/NASH-driven HCC. Panel A depicts that representative ATF6-positive staining in human normal, NAFLD and HCC tissue from a cohort of patient samples. It was found that ATF6 positivity was cytoplasmic in normal liver, both cytoplasmic and nuclear in NAFLD, and strictly nuclear in HCC. Panel B depicts western blotting from HCC patient samples, showing ATF6p50 expression was indeed increased in tumor vs. nontumor samples, especially in patients with a NASH HCC etiology (red box). Panel C depicts cancer microarray database and data-mining (Oncomine) found an association between increased ATF6 mRNA expression in human HCC vs. normal tissue.

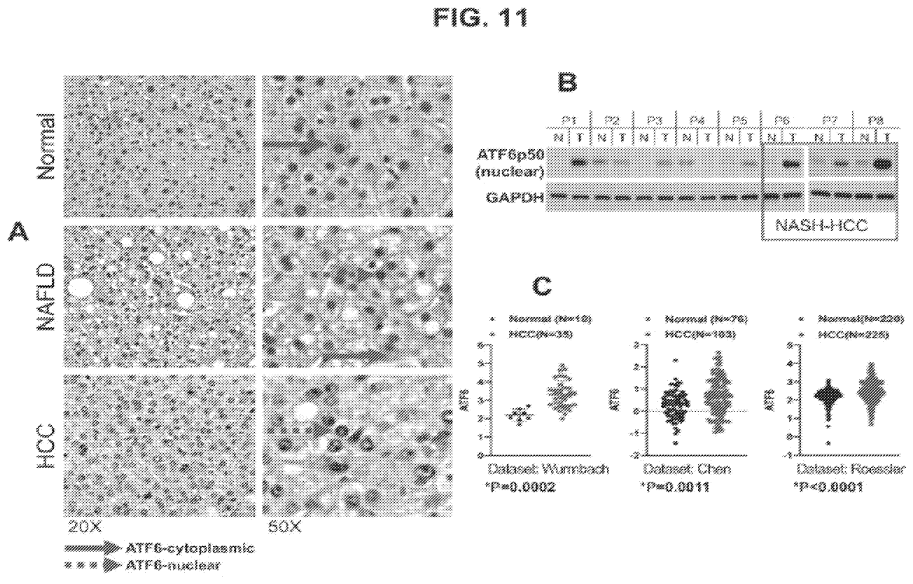

FIG. 12 depicts a model for how the UPR transducer ATF6 drives HCC in response to metabolic stress.

FIG. 13 depicts that MUP-uPA mice crossed with Atf6−/− or TG-ATF6p50 mice develop fewer or more liver tumors, respectively, and treatment of MUP-uPA mice with GalNac-ASO-Atf6 reduces liver damage and induction of UPR markers. Panel A depicts Atf6−/−/MUP-uPA mice fed high-fat diet for 55 wks develop less tumors than UP-uPA mice and Panels B-C depicts mice TG-ATF6p50/MUP-uPA mice fed high-fat diet for 24 wks develop more tumors than wild-type or MUP-uPA mice. Panels D-E depicts 7-week-old mice sacrificed after GalNac-ASO-Atf6 delivery (5 mg/kg s.c. 2/wk, 5×).

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

In this application, the use of the singular includes the plural unless specifically stated otherwise. It must be noted that, as used in the specification and the appended claims, the singular forms “a,” “an,” and “the” include plural referents unless the context clearly dictates otherwise. Furthermore, use of the term “including” as well as other forms, such as “include,” “includes,” and “included,” is not intended to be limited solely to the recited items. The section headings used herein are for organizational purposes only and are not to be construed as limiting the subject matter described.

“Alkyl” as used herein refers to a fully saturated straight or branched hydrocarbon group. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, an alkyl group can be unsubstituted or substituted.

“Alkoxy” refers to the group R—O—, where R is alkyl as defined herein. A non-limiting list of alkoxys includes, byway of example, methoxy, ethoxy, n-propoxy, iso-propoxy, n-butoxy, tert-butoxy, sec-butoxy, n-pentoxy, n-hexyloxy, 1,2-dimethylbutoxy, and the like. The alkoxy group can be designated as “C₁-C₅-alkoxy” or similar designations. In some embodiments, an alkoxy can be unsubstituted or substituted.

“Aryl” or “Ar” as used herein refers to an aromatic ring system containing 6, 10 or 14 carbon atoms that can contain a single ring, two fused rings or three fused rings, such as phenyl, naphthalenyl and phenanthrenyl. In one aspect, aryl group can have 6 or 10 carbon atoms (i.e., C₆ or C₁₀ aryl). In some embodiments, an aryl is phenyl. In some embodiments, an aryl is naphthyl. In some embodiments, an aryl is a C₆ or C₁₀aryl. In some embodiments, an aryl group can be unsubstituted or substituted.

“Cycloalkyl” as used herein refers to a monocyclic or polycyclic hydrocarbon including at least one fully or partially saturated ring (i.e., non-aromatic ring), wherein each of the atoms forming the ring is a carbon atom. In some embodiments, cycloalkyls are spirocyclic or bridged compounds. In some embodiments, cycloalkyls include at least one partially saturated ring fused with an aromatic ring (e.g., 1,2,3,4-tetrahydronaphthalenyl), and the point of attachment is at a carbon of either ring. Cycloalkyl groups include groups having from 3 to 10 ring atoms. The cycloalkyl group can be designated as “C₃-C₁₀-cycloalkyl” or similar designations. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl, bicycle[1.1.1]pentyl, 2,3-dihydro-1H-indene and 1,2,3,4-tetrahydronaphthalenyl. In some embodiments, a cycloalkyl is a C₃-C₁₀-cycloalkyl. In some embodiments, a cycloalkyl is a C₃-C₉-cycloalkyl.

“Halo” or “halogen” refers to a fluoro, chloro, bromo or iodo group. In some embodiments, halogen or halo is fluoro, chloro, or bromo. In some embodiments, halogen or halo is fluoro.

The term “haloalkyl” refers to an alkyl group with one or more halo substituents, or one, two, or three halo substituents. Examples of haloalkyl groups include —CF₃, —(CH₂)F, —CHF₂, —CH₂Br, —CH₂CF₃, and —CH₂CH₂F.

“Heteroaryl” as used herein refers to a monocyclic or fused multicyclic aromatic ring system and having at least one heteroatom in the ring system, including but not limited to nitrogen, oxygen and sulfur. Illustrative examples of heteroaryl groups include monocyclic heteroaryls and bicyclic heteroaryls. Monocyclic heteroaryls include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, 1,3,5-triazinyl, 1,3,4-oxadiazolyl, thiadiazolyl, and 1,2,5-oxadiazolyl. Bicyclic heteroaryls include, but are not limited to, indolizinyl, indolyl, benzofuranyl, benzothiophene (i.e., benzothiofuranyl), indazolyl, benzimidazolyl, purinyl, quinolinyl, isoquinolinyl, cinnolinyl, phthalazinyl, quinazolinyl, quinoxalinyl, 1,8-naphthyridinyl, and pteridinyl. In some embodiments, a heteroaryl contains 1-4 nitrogen atoms in the ring. In some embodiments, a heteroaryl has 5 to 10 ring members or 5 to 9 ring members. The heteroaryl group can be designated as “5-10 membered heteroaryl,” “5-9 membered heteroaryl,” or similar designations. In some embodiments, a heteroaryl can be an optionally substituted C₁-C₁₃ five-, six-, seven, eight-, nine-, ten-, up to 14-membered monocyclic, bicyclic, or tricyclic ring system including 1 to 5 heteroatoms selected from nitrogen, oxygen and sulfur. In some embodiments, a heteroaryl can be an optionally substituted C₁-C₈ five-, six-, seven, eight-, or nine-membered monocyclic, or bicyclic ring system including 1 to 4 heteroatoms selected from nitrogen, oxygen and sulfur. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, the C₁-C₄ 5-membered heteroaryl is furanyl, thienyl, 1,2,4-thiadiazolyl, 1,2,3-thiadiazolyl, isothiazolyl, thiazolyl, imidazolyl, pyrazolyl, isoxazolyl, oxazolyl, pyrrolyl, triazolyl, or tetrazolyl. In some embodiments, the heteroaryl is a C₃-C₅ 6-membered heteroaryl. In some embodiments, the C₃-Cₛ 6-membered heteroaryl is pyridinyl, pyrimidinyl, pyrazinyl, pyridazinyl, or triazinyl. In some embodiments, a heteroaryl can be an optionally substituted C₆-C₉ ten-membered bicyclic ring system including 1 to 4 nitrogen atoms. In some embodiments, a heteroaryl can be an optionally substituted C₄-C₈ eight-, or nine-membered bicyclic ring system including 1 to 4 heteroatoms selected from nitrogen, oxygen and sulfur. In some embodiments, a heteroaryl can be an optionally substituted C₅-C₈ nine-membered bicyclic ring system including 1 to 4 heteroatoms selected from nitrogen, oxygen and sulfur. In some embodiments, a heteroaryl can be an optionally substituted C₅-C₈ nine-membered bicyclic ring system including 1 to 4 nitrogen atoms. In some embodiments, bicyclic heteroaryl is a C₆-C₉heteroaryl. In some embodiments, a heteroaryl group can be unsubstituted or substituted.

“Heterocycle”, “heterocyclic”, or “heterocyclyl” as used herein refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system and optionally containing one or more unsaturated bonds situated in such a way, however, that a fully delocalized pi-electron system does not occur in the monocyclic ring or in at least one ring of the bicyclic or tricyclic ring system. The heteroatom(s) is an element other than carbon including, but not limited to, oxygen, sulfur, and nitrogen. When composed of two or more rings, the rings can be joined together in a fused, bridged, or spiro fashion where the heteroatom(s) can be present in any ring in the ring system. In some embodiments, a heterocycloalkyl can be an C₂-C₁₂ three-, four-, five-, six-, seven-, eight-, nine-, ten-, up to 13-membered monocyclic, bicyclic, or tricyclic ring system including 1 to 5 heteroatoms selected from nitrogen, oxygen and sulfur. In some embodiments, the heterocyclyl can be a C₂-C₆ three-, four-, five-, six-, or seven-membered monocyclic ring including 1 to 5 heteroatoms selected from nitrogen, oxygen and sulfur. In some embodiments, the heterocyclyl can be a C₂-C₁₀ four-, five-, six-, seven-, eight-, nine-, ten- or eleven-membered bicyclic ring system including 1 to 5 heteroatoms independently selected from nitrogen, oxygen and sulfur. In some embodiments, the heterocyclyl can be a C₇-C₁₂ 12- or 13-membered tricyclic ring system including 1 to 5 heteroatoms selected from nitrogen, oxygen and sulfur. In some embodiments, the heteroatom(s) of six membered monocyclic heterocyclyls are independently selected from one to three of oxygen, nitrogen and sulfur, and the heteroatom(s) of five membered monocyclic heterocyclyls are independently selected from oxygen, nitrogen and sulfur. In some embodiments, the heterocycloalkyl is pyrrolidinyl, tetrahydrofuranyl, tetrahydrothienyl, tetrahydropyranyl, tetrahydrothiopyranyl, piperidinyl, morpholinyl, thiomorpholinyl, or piperazinyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1 or 2 nitrogen atoms in the ring. In some embodiments, a heterocycloalkyl can be aziridinyl, azetidinyl, tetrahydrofuranyl, 1,3-dioxinyl, 1,3-dioxanyl, 1,4-dioxanyl, 1,2-dioxolanyl, 1,3-dioxolanyl, 1,3-oxathianyl, 1,4-oxathianyl, 1,3-oxathiolanyl, 1,3-dithiolyl, 1,3-dithiolanyl, 1,4-oxathianyl, tetrahydro-1,4-thiazinyl, imidazolinyl, imidazolidinyl, isoxazolinyl, isoxazolidinyl, isoindolinyl, indolinyl, oxazolinyl, oxazolidinyl, thiazolinyl, thiazolidinyl, morpholinyl, oxiranyl, piperidinyl, piperazinyl, pyrrolidinyl, pyrazolinyl, pyrazolidinyl, tetrahydropyranyl, tetrahydrothiopyranyl, 1,4-diazabicyclo[2.2.2]octane, 1,4-diazabicyclo[3.1.1]heptane, 2-azaspiro[3,3]heptane, 2,6-diazaspiro[3,3]heptane, tetrahydroquinolinyl, 1,2,3,4-tetrahydroisoquinolinyl, 1,2,3,4-tetrahydro-2,6-naphthyridinyl, 1,2,3,4-tetrahydro-2,7-naphthyridinyl, 1,2,3,4-tetrahydro-1,7-naphthyridinyl, 1,2,3,4-tetrahydro-1,6-naphthyridinyl, 5,6,7,8-tetrahydropyrido[2,3-d]pyrimidinyl, 5,6,7,8-tetrahydropyrido[3,4-d]pyrimidinyl, [1,3]dioxolo[4,5-c]pyridinyl, [1,3]dioxolo[4,5-b]pyridinyl, [1,3]dioxolo[4,5-d]pyrimidinyl or 3,4-methylenedioxyphenyl. In some embodiments, a substituted heterocyclyl can be oxazolidinonyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. When one or more substituents are present on the heterocyclyl group, the substituent(s) can be bonded at any available carbon atom and/or heteroatom. In some embodiments, a heterocyclyl group can be unsubstituted or substituted.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “therapeutically effective amount” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival.

As used herein, “treatment” or“treating” refers to an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of an individual.

The terms “about” or “approximately,” as used herein, generally mean within an acceptable error range for the particular value as determined by one of ordinary skill in the art, which will depend in part upon how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” can mean within an order of magnitude, within 5-fold, and more preferably within 2-fold of a value.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### INTRODUCTION

The endoplasmic reticulum (ER) is a ubiquitous organelle responsible for folding and trafficking of proteins destined for the cellular intramembrane system and for secretion, lipid biosynthesis and Ca2+ homeostasis. Changes in the extracellular and intracellular environments cause accumulation of misfolded proteins in the ER (i.e., ER stress), which activates three ER-localized adaptive unfolded protein response (UPR) sensors: 1) PERK to attenuate protein synthesis; 2) IRE1 to initiate splicing of XBP1 mRNA to generate a potent transcription factor, XBP1s; and 3) ATF6 processing by S1P and S2P to release an active transcription factor, ATF6p50, that traffics to the nucleus. If ER protein folding homeostasis is not resolved, cells activate apoptosis, primarily through the transcription factor CHOP, downstream of the PERK and ATF6 UPR pathways. Significantly, the UPR is a negative prognosticator for outcome in solid tumors that is also activated in disease-afflicted mouse and human livers. According to the present disclosure, ER stress is linked to Non-alcoholic Fatty Liver Disease (NAFLD), characterized by abnormal lipid accumulation in the liver that can progress to steatohepatitis (NASH) with inflammation, hepatocyte ballooning, cell death and fibrosis, cirrhosis and hepatocellular carcinoma (HCC), the most common primary liver cancer and a leading cause of cancer-related deaths. It is unknown whether/how ER stress impacts NASH and HCC development. Extensive studies on PERK and IRE1 demonstrate they promote tumor growth, angiogenesis, inflammation, metastasis and resistance to chemotherapy; however, little is known about ATF6 in cancer and how an ER stress modulator might inhibit or alter cellular function in NASH or HCC.

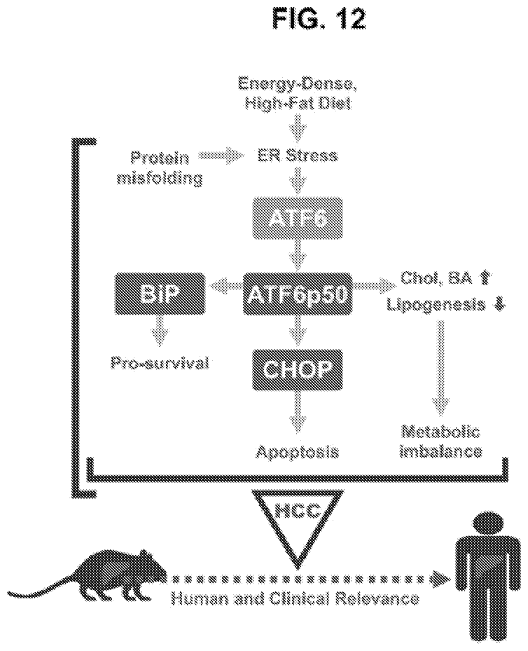

ATF6 accumulation and activation is associated with HCC in humans. In some embodiments, nuclear ATF6p50 is elevated in human HCC tumor compared to non-tumor tissues. According to the present disclosure, ATF6 plays a pivotal role in HCC development: 1) Expression of activated ATF6p50 spontaneously promotes HCC in mice; and 2) Deletion of ATF6 attenuates HCC in carcinogen+high-fat diet (HFD) and MUP-uPA+HFD murine models. (See, e.g., FIG. 12).

In some embodiments, aberrant ATF6 cleavage or activation drives HCC progression through UPR activation and a metabolic shift to alter sterol/lipid metabolism, both promoting hepatocyte death. Data, according to some example embodiments, indicate that ATF5p50 drives Chop mRNA expression and protein accumulation, suggesting that chip deletion can mitigate the oncogenic affect of ATF6p50. In some example embodiments, ATF6p50 promotes cholesterol synthesis and will target cholesterol signaling. In some embodiments, activated NRF2Act-Hep drives the progression of HCC in ATF6-null subjects. Activated NRF2 accelerates HCC progression, similar to ATF6. In some embodiments, inactivation of ATF6 prevents HCC (i.e., HCC and/or NASH-HCC). Hepatocyte-specific inactivation of ATF6 will attenuate HCC. In some cases, the NRF2 and ATF6 act through a common, parallel or partially overlapping pathway via a downstream effector.

In some embodiments, hepatocyte-specific inactivation of ATF6 can attenuate NASH-HCC. In some embodiments, the role of ATF6 in HCC is defined by Atf6 deletion or ATF6p50 expression in HCC cell lines and organoids from MUP-uPA, NRF2Act-Hep/MUP-uPA, ATF6p50Hep/MUP-uPA subjects for deep transcriptome/metabolome phenotyping and intrasplenic injection into HFD-fed wildtype subjects to monitor HCC progression. In some embodiments, ATF6 activation status can alter ER protein misfolding and whether targeting ATF6 prevents HCC. In some cases, activation of ATF6 can be oncogenic and responsible for metabolic switches that promote human HCC progression.

ATF6p50 may impact phenotypes of human NASH-related HCC organoids. Fully-characterized human organoids can be harvested to determine whether targeting ATF6 and/or cholesterol biosynthesis prevents tumor growth in vitro, potentially synergistically. Tumorigenic potential, transcriptomic and metabolomic changes can be assessed by orthotopic injection into Nu/Nu mice. In some embodiments, Nanostring DSP with next generation sequencing (NGS) is performed to probe transcriptome changes associated with ATF6p50 expression and liver disease progression and transformation to HCC in humans. In some embodiments, oncogenic factor ATF6, a key regulator of ER stress, protein homeostasis, cholesterol and/or lipid metabolism, is implicated in NASH-driven HCC upon aberrant activation, which leads to new biomarkers for HCC progression and therapeutic strategies for intervention. In some cases, targeting ATF6 can selectively interfere with growth of hepatocellular precancerous cells.

The present disclosure provides methods and compositions for identifying agents that inhibit ATF6 in hepatocytes as a therapeutic strategy in chronic liver disease and liver cancer. This includes, but is not limited to, the development and testing of Atf6-specific short, synthetic, single-stranded oligodeoxynucleotides (ASOs) in vitro and in vivo and future screening for ATF6 inhibitors.

### Compounds and Compositions

Disclosed herein, in some embodiments, are methods useful for treating a subject with a hepatic condition. In some embodiments, the method includes administering a pharmaceutical composition to a subject for treating a hepatic condition. In some embodiments, the pharmaceutical composition includes a therapeutically effective amount of a synthetic activating transcription factor inhibitor (ATFi). In some embodiments, the synthetic ATFi modulates signaling of an activating transcription factor (ATF) in a cell via an interference with a cellular pathway. In some embodiments, synthetic ATFi enters hepatocytes. In some embodiments, the synthetic ATFi modulates signaling of an activating transcription factor (ATF) in a hepatocyte. In some embodiments, the method includes administering a pharmaceutical composition that modulates a hepatocyte. In some embodiments, the synthetic ATFi modulates the function of the endoplasmic reticulum. In some embodiments, the synthetic ATFi modulates the function of the Golgi apparatus.

In some embodiments, interference with the cellular pathway includes sequestering an ATF protein as a transmembrane protein in an endoplasmic reticulum, preventing trafficking of the ATF protein from the endoplasmic reticulum to a Golgi apparatus, cleaving the ATF protein via one or more proteases, or blocking trafficking of a soluble and active ATF fragment to a nucleus of the cell, or a combination thereof. In some embodiments, interference with the cellular pathway includes sequestering an ATF protein as a transmembrane protein in an endoplasmic reticulum. In some embodiments, interference with the cellular pathway includes preventing trafficking of the ATF protein from the endoplasmic reticulum to a Golgi apparatus. In some embodiments, interference with the cellular pathway includes cleaving the ATF protein via one or more proteases. In some embodiments, interference with the cellular pathway includes blocking trafficking of a soluble and active ATF fragment to a nucleus of the cell.

In some embodiments, the method includes administering a pharmaceutical composition to a subject for treating a hepatic condition, as disclosed herein. In some cases, the hepatic condition is non-alcoholic fatty liver disease (NAFLD). In some cases, the hepatic condition is non-alcoholic steatohepatitis (NASH). In some cases, the hepatic condition is hepatocyte ballooning. In some cases, the hepatic condition is hepatocyte cell death and fibrosis. In some cases, the hepatic condition is cirrhosis and hepatocellular carcinoma.

### Compounds

In an embodiment of the present disclosure, a method of treating a subject having a condition comprises administering a pharmaceutical composition comprising a therapeutically effective amount of a compound. In some embodiments, the compound can be a small-molecule inhibitor. In some embodiments, the compound is a synthetic ATFi. In some embodiments, the method of treating a subject having a condition comprises oral administration. In some embodiments, the method of treating a subject having a condition comprises subcutaneous injection. In some embodiments, the method of treating a subject having a condition comprises intravenous injection. In some embodiments, the method of treating a subject having a condition comprises intramuscular injection. In some embodiments, the activating transcription factor (ATF) comprises ATF1, ATF2, ATF3, ATF4, ATF5, ATF6 or ATF7. ATF's are involved in cell proliferation, apoptosis, differentiation and inflammation-related pathological processes. Chen M, Emerging Roles of Activating Transcription Factor (ATF) family members in tumourigenesis and immunity, Genes & Diseases, 2022, 9(4): 981-999 (available online: 3 Jun. 2021).

In some embodiments, the condition comprises a hepatic condition. In some embodiments, the hepatic condition comprises non-alcoholic acute steatohepatitis (NASH), non-alcoholic fatty liver disease (NAFLD), hepatitis, cirrhosis, alcoholic hepatitis, hemochromatosis, jaundice, enlargement of the liver, ascites fluid in the abdomen, encephalopathy, drug-induced liver disease, hepatocellular carcinoma, cholangicarcinoma, metastatic liver cancer, or liver cancer.

In some embodiments, a method of treating a subject having a hepatic condition comprises administering a pharmaceutical composition comprising a therapeutically effective amount of a compound, wherein the compound has a structure of Formula (I):

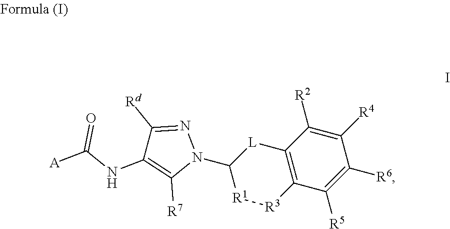

or a pharmaceutically acceptable salt thereof, wherein:
R^{d} is H or C₁-C₆ alkyl;
R¹ is C₁-C₆ alkyl, C₃-C₈ cycloalkyl, or C₁-C₆ haloalkyl;
L is —CH₂— or is absent;
-- between R¹ and R³ is a bond or is absent;
R², R³, R⁴, R⁵, and R⁶ are each independently H, halo, CN, C₁-C₆ alkyl, or C₁-C₆ haloalkyl, wherein at least two of R², R³, R⁴, R⁵, and R⁶ are halo, CN, C₁-C₆ alkyl, or C₁-C₆ haloalkyl, and/or wherein one of R², R³, R⁴, R⁵, and R⁶ is CN; or R², R⁴, R⁵, and R⁶ are each independently H, halo, CN, C₁-C₆ alkyl, or C₁-C₆ haloalkyl and --- between R¹ and R³ is a bond, such that R³ is taken together with R¹ and the atoms to which they are attached to form a 5- or 6-membered carbocyclic ring, wherein the 5- or 6-membered carbocyclic ring is unsubstituted or substituted with one to three groups selected from the group consisting of halo, CN, —OH, C₁-C₆ alkyl, and C₁-C₆ haloalkyl;
A is

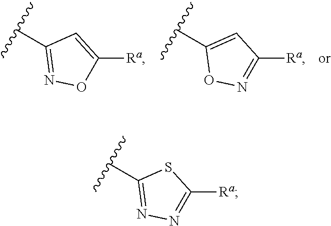

R^{a} is 5- or 6-membered heteroaryl, wherein the 5- or 6-membered heteroaryl is unsubstituted or substituted with one to four groups selected from the group consisting of OH, halo, C₁-C₆ alkyl and C₁-C₆ alkoxy. In some embodiments, A is

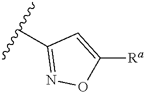

and R^{a} is 2-furyl or 2-thiofuryl,
(i) L is absent, and R¹ is C₁-C₆ alkyl;
(ii) --- is a bond, such that R³ is taken together with R¹ and the atoms to which they are attached to form a 5- or 6-membered carbocyclic ring, provided that when the 5-membered carbocyclic ring is formed, at least one of R², R⁴, R⁵, and R⁶ is halo, CN, C₁-C₆ alkyl, or C₁-C₆ haloalkyl;
(iii) one of R², R³, R⁴, R⁵, and R⁶ is CN;
(iv) R⁴ and R⁵ are each independently Cl, Br, I, CN, C₁-C₆ alkyl, or C₁-C₆ haloalkyl;
(v) R² and R³ are each Cl;
(vi) at least one of R², R³, R⁴, R⁵, and R⁶ is F, Br, I, CN, or C₁-C₆ haloalkyl and R^{a} is 2-thiofuryl;
(vii) R⁷ is H, C₁-C₆ alkyl or C₁-C₆ haloalkyl;
(viii) R^{d} is C₁-C₆ alkyl, R⁷ is H; or
(ix) R⁷ is C₁-C₆ alkyl, and R^{d} is H.

In some embodiments, a method of treating a subject having a hepatic condition comprises administering a pharmaceutical composition comprising a therapeutically effective amount of a compound, wherein the compound has a structure of Formula (II):

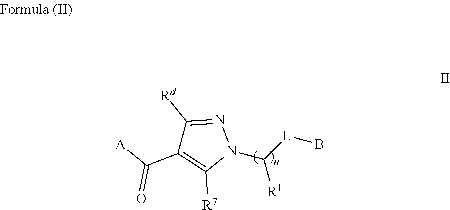

or a pharmaceutically acceptable salt thereof, wherein:
R¹ is H, C₁-C₆ alkyl, C₃-C₈ cycloalkyl, or C₁-C₆ haloalkyl;
n is 0 or 1;
L is —CH₂— or is absent;
B is C₃-C₆ cycloalkyl, 3-6 membered heterocycle, or 5-6 membered heteroaryl, wherein the C₃-C₆ cycloalkyl and 5-6 membered heteroaryl are each independently optionally substituted with one or more groups selected from the group consisting of halo, CN, —OH, —NH₂, C₁-C₆ alkyl, C₁-C₆ alkoxy and C₁-C₆ haloalkyl;
A is

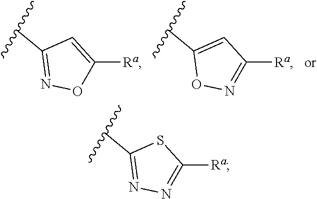

R^{a} is 5- or 6-membered heteroaryl optionally substituted with one to four halo. In some embodiments, A is

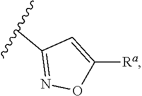

(i) B is C₃-C₆ cycloalkyl, optionally substituted with one or more groups selected from the group consisting of halo, CN, —OH, —NH₂, C₁-C₆ alkyl, C₁-C₆ alkoxy and C₁-C₆ haloalkyl;
(ii) B is 3-6 membered heterocycle having one or more annular heteroatoms, wherein the 3-6 membered heterocycle is optionally substituted with one or more groups selected from the group consisting of halo, CN, —OH, —NH₂, C₁-C₆ alkoxy and C₁-C₆ haloalkyl, and wherein the one or more annular heteroatoms are nitrogen;
(iii) B is

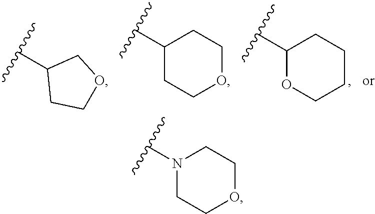

each of which is optionally substituted with one or more groups selected from the group consisting of halo, CN, —OH, —NH₂, C₁-C₆ alkyl, C₁-C₆ alkoxy and C₁-C₆ haloalkyl, n is 1, R¹ is CH₃, and L is absent;
(iv) B is

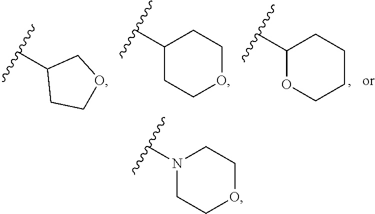

each of which is substituted with one or more groups selected from the group consisting of halo, CN, —OH, —NH₂, C₁-C₆ alkyl, C₁-C₆ alkoxy and C₁-C₆ haloalkyl;
(v) B is

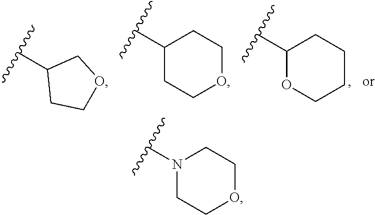

each of which is optionally substituted with one or more groups selected from the group consisting of halo, CN, OH, —NH₂, C₁-C₆ alkyl, C₁-C₆ alkoxy and C₁-C₆ haloalkyl, and R^{a} is

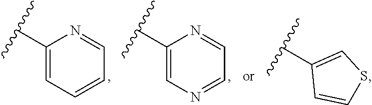

each of which is optionally substituted with one to four halo;
(vi) B is 4-6 membered bridged heterocycle, optionally substituted with one or more groups selected from the group consisting of halo, CN, —OH, —NH₂, C₁-C₆ alkyl, C₁-C₆ alkoxy and C₁-C₆ haloalkyl; or
(vii) B is 5-membered heteroaryl or

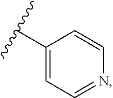 [SMILES: *c1ccncc1]

each of which is optionally substituted with one or more groups selected from the group consisting of halo, CN, —OH, —NH₂,
C₁-C₆ alkyl, C₁-C₆ alkoxy and C₁-C₆ haloalkyl; R⁷ is H, C₁-C₆ alkyl or C₁-C₆ haloalkyl; and R⁸ is H or C₁-C₆ alkyl.

In some embodiments, a method of treating a subject having a hepatic condition comprises administering a pharmaceutical composition comprising a therapeutically effective amount of a compound, wherein the compound has a structure of Formula (III):

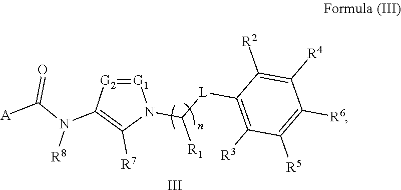

or a pharmaceutically acceptable salt thereof, wherein: one of G₁ and G₂ is N and one of G₁ and G₂ is CR^{d}, wherein R^{d} is H or C₁-C₆ alkyl;
R¹ is H, C₁-C₆ alkyl, or C₃-Cₛ cycloalkyl; or Riis H, C₁-C₆ alkyl, C₃-Cₛ cycloalkyl, or C₁-C₆ haloalkyl;
R⁸ is H or C₁-C₆ alkyl;
n is 0 or 1;
L is CH₂ or is absent;
R², R³, R⁴, R⁵, and R⁶ are each independently H, halo, CN, C₁-C₆ alkyl, or C₁-C₆ haloalkyl;
or R², R⁴, R⁵, and R⁶ are each independently H, halo, CN, C₁-C₆ alkyl, or C₁-C₆ haloalkyl and R³ is taken together with an R¹ and the atoms to which they are attached to form a 5- or 6-membered cycloalkyl ring, wherein the 5- or 6-membered cycloalkyl ring is unsubstituted or substituted with one to three groups each independently selected from the group consisting of halo, CN, OH, C₁-C₆ alkyl, and C₁-C₆ haloalkyl;
   A is -L¹-5-6 membered heteroaryl optionally substituted with one to three substituents each independently selected from R^{a}, R^{b} R^{c} and R^{e};
   L¹ is CH═CH or absent;
R^{a} and R^{b} are each independently H, C₁-C₆ alkyl, —C(O)C₁-C₆ alkyl, 6-membered aryl, or 5- or 6-membered heteroaryl, wherein each C₁-C₆ alkyl, —C(O)C₁-C₆ alkyl, 6-membered aryl, or 5- or 6-membered heteroaryl of R^{a} and R^{b} are unsubstituted or substituted with one to four groups selected from the group consisting of OH, halo, and C₁-C₆ alkyl, or unsubstituted or substituted with one to four groups selected from OH, halo, C₁-C₆ alkyl and C₁-C₆ alkoxy;
R^{c} is C₁-C₆ alkyl, —C(O)C₁-C₆ alkyl, 6-membered aryl, or 5- or 6-membered heteroaryl, wherein each C₁-C₆ alkyl, —C(O)C₁-C₆ alkyl, 6-membered aryl, or 5- or 6-membered heteroaryl of Reis unsubstituted or substituted with one to four groups selected from the group consisting of OH, halo, and C₁-C₆ alkyl;
R^{e} is H, or C₁-C₆ alkyl; and
R⁷ is H, C₁-C₆ alkyl or C₁-C₆ haloalkyl. In some embodiments, R^{d} is C₁-C₆ alkyl, and R⁷ is H. In some embodiments, R⁷ is C₁-C₆alkyl, and R^{d} is H. In some embodiments, R³ is taken together with R¹ and the atoms to which they are attached to form a 5- or 6-membered cycloalkyl ring. In some embodiments, at least two of R², R³, R⁴, R⁵, and R⁶ are other than H. In some embodiments, one of R², R³, R⁴, R⁵, and R⁶ is cyano. In some embodiments, A is

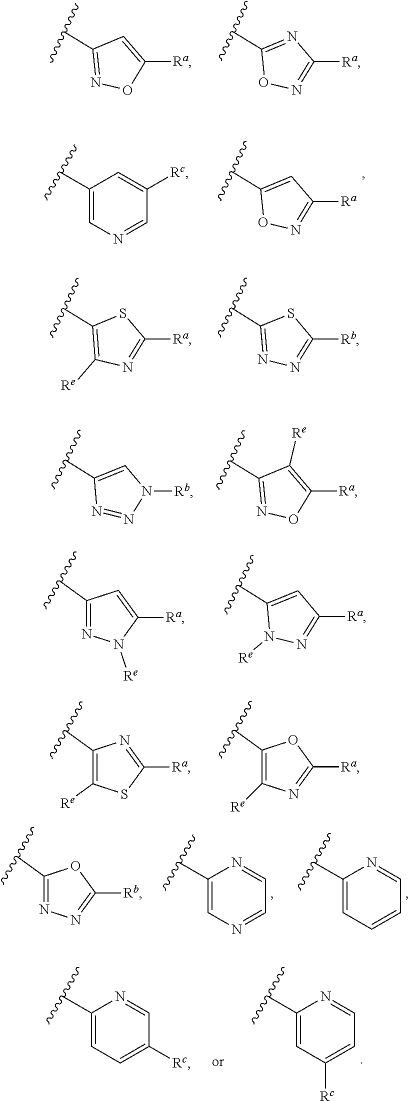

In some embodiments, A is

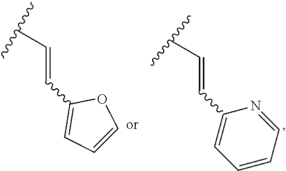 [SMILES: *C=Cc1ccco1 ; *C=Cc1ccccn1]

wherein [symbol] indicates that

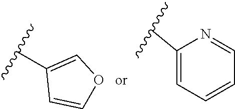

is attached in either an E or Z configuration. In some embodiments, L¹ is CH═CH. In some embodiments, L¹ is absent. In some embodiments, A is

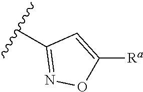

and R^{a} is H, methyl, ethyl, n-Pr, i-Pr, i-Bu, 2-thiofuryl, 2-furyl, unsubstituted phenyl, 2-methoxyphenyl, 3-methoxyphenyl, 3,4-dimethoxyphenyl, 3-hydroxyphenyl, 4-hydroxyphenyl, 4-fluorophenyl, or 2,4-dichlorophenyl,
(i) G₂ is N;
(ii) n is 1, L is absent, and R¹ is C₁-C₆ alkyl;
(iii) n is 0, L is absent, and at least one of R², R³, R⁴, R⁵, and R⁶ is halo, CN, or C₁-C₆ haloalkyl;
(iv) n is 1, and R³ is taken together with R¹ and the atoms to which they are attached to form a 5- or 6-membered cycloalkyl ring;
(v) one of R², R³, R⁴, R⁵, and R⁶ is CN; and
(vi) R⁴ and R⁵ are each independently Cl, Br, I, CN, C₁-C₆ alkyl, or C₁-C₆ haloalkyl;
(vii) one of R^{d} and R⁷ is C₁-C₆ alkyl;
(viii) R² and R³ are each Cl; or
(ix) at least one of R², R³, R⁴, R⁵, and R⁶ is F, Br, I, CN, or C₁-C₆ haloalkyl and R^{a} is H or 2-thiofuryl. In some embodiments, A is

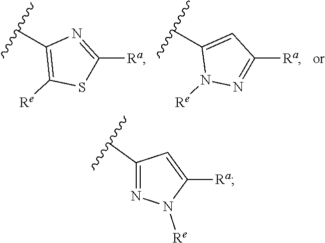

n is 1, and R¹ is other than H. In some embodiments, A is

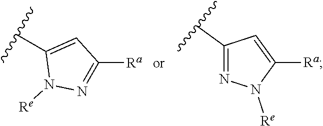

n is 0; L is absent, and R^{a} is other than H, In some embodiments, A is

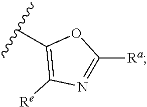

R^{e} is methyl; and R^{a} is other than unsubstituted phenyl. In some embodiments, A is

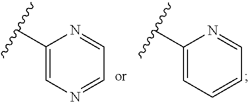 [SMILES: *c1cnccn1 ; *c1ccccn1]

n is 1; L is absent, and R¹ is other than H.

Compounds described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically-labeled compound. Each chemical element as represented in a compound structure may include any isotopic distribution of said element. For example, in a compound structure a hydrogen atom can be explicitly disclosed or understood to be present in the compound. At any position of the compound that a hydrogen atom can be present, the hydrogen atom can be an isotopic distribution of hydrogen, including but not limited to protium (¹H) and deuterium (²H) in proportional amounts to those usually found in nature and in proportional amounts different to those usually found in nature. Thus, reference herein to a compound encompasses all potential isotopic distributions for each atom unless the context clearly dictates otherwise. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays.

In one aspect, substitution with isotopes such as deuterium affords certain therapeutic advantages resulting from greater metabolic stability, such as, for example, increased in vivo half-life or reduced dosage requirements. In some embodiments, an atom in one position of the compound has an isotopic distribution for a chemical element in proportional amounts different to those usually found in nature (remainder atoms having an isotopic distribution for a chemical element in proportional amounts to those usually found in nature). In some embodiments, atoms in at least two positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least three positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least four positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least five positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least six positions of the compound independently have an isotopic distribution for a chemical element in proportional amounts different to those usually found in nature (remainder atoms having an isotopic distribution for a chemical element in proportional amounts to those usually found in nature). The compounds are described in Example 3.

In certain aspects, a compound can be an inhibitor. In some embodiments, the inhibitor can be an ATFi. In some embodiments, the ATFi can be an antisense oligonucleotide.

In some embodiments, the antisense oligonucleotide can be transported in a synthetic lipid nanoparticle (LNP) carrier. Synthetic LNPs prevent degradation of the antisense oligonucleotides from RNases and thereby can facilitate transport into the cytoplasm of a hepatocyte. LNPs also permit the use of minimally modified antisense oligonucleotide backbones that primarily contained native 2′-hydroxyl groups. In some embodiments the antisense oligonucleotide may comprise a 2′-modification. 2′-modifications increase the stability of the antisense oligonucleotide. 2′-modified antisense oligonucleotides are able to conjugate targeting domains to naked antisense oligonucleotides, such as siRNAs, and thereby avoid use of LNPs. In some embodiments, the conjugate can be a trimer of N-acetylgalactosamine (GalNAc).

In some embodiments, the antisense oligonucleotide knocks down an endonuclease transcript and reduces levels of a target protein via binding to a target mRNA. In some embodiments, the synthetic antisense oligonucleotide is conjugated to a triantennary N-acetyl galactosamine (GalNAc). In some embodiments, the GalNAc targets and binds to an asialoglycoprotein receptor, which is highly expressed on hepatocytes, resulting in rapid endocytosis. The mechanism includes introducing sufficient amounts of antisense oligonucleotides into a cytoplasm of a cell to induce target-selective responses in vivo. In some embodiments, the antisense oligonucleotide comprises a short interfering RNA (siRNA) molecule.

In some embodiments, the compound, salt thereof, or composition inhibits the ATF6 pathway, ATF6, or ATF6a with an IC50 of less than about 10 μM, such as less than about 5 μM, 2 μM, 1 μM, 900 nM, 800 nM, 700 nM, or 600 nM. In some embodiments, the compound, salt thereof, or composition inhibits the ATF6 pathway, ATF6, or ATF6a with an IC50 between about 10 nM and 5 μM, such between about 50 nM and 2 μM, 100 nM and 1 μM, or 20 nM and 1 μM. The half maximal inhibitory concentration (IC50) is a measure of the effectiveness of a substance in inhibiting a specific biological or biochemical function. The IC50 is a quantitative measure that indicates how much of an inhibitor is needed to inhibit a given biological process or component of a process such as an enzyme, cell, cell receptor or microorganisms by half. Methods of determining IC50 in vitro and in vivo are known in the art.

The present compounds or salts thereof are believed to be effective for treating a variety of conditions and disorders, such as conditions wherein ATF6-activated transcription targets play a role in the pathogenesis or development of the condition. For example, in some embodiments, the present compounds and compositions may be used to treat viral infection, cancer, a neurodegenerative condition, or a vascular condition, such as a cardiovascular disease. In some embodiments, the condition is viral infection, hereditary cerebellar atrophy and ataxia, or Alzheimer's disease. In some embodiments, the disease is type 2 diabetes mellitus or diabetic nephropathy. In some embodiments, the disease is myocardial atrophy, heart failure, atherosclerosis, or ischemia, such as ischemic heart disease or cerebral ischemia.

In some embodiments, a compound or salt thereof described herein, or a composition described herein may be used in a method as either a stand-alone therapy, or as a conjunctive therapy with other agents that are either palliative (e.g., agents that relieve the symptoms of the disorder to be treated), and/or agents that target the etiology of the disorder. For example, the administration to a subject of a composition that increases the expression of ATF6 may be carried out in conjunction with the administration of L-DOPA, dopamine agonists, monoamine oxidase B inhibitors, or any other composition useful in the treatment of a neurodegenerative disease, such as Parkinson's disease. Overexpression of the active ATF6 transcription factor in the heart improves cardiac performance in mouse models of ischemic heart disease, through a mechanism involving ATF6-dependent regulation of the antioxidant gene, catalase. Similarly, overexpression of the active ATF6 transcription factor in the liver improves insulin sensitivity in obese mice. These results indicate that ATF6 activation offers a unique therapeutic opportunity to ameliorate ER proteo-stasis defects implicated in diverse diseases.

The ATF6a pathway also plays a role in stress-induced lipid accumulation. p50ATF6 interacts with the nuclear form of SREBP-2, thereby antagonizing SREBP-2-regulated transcription of lipogenic genes and lipid accumulation in cultured hepatocytes and kidney cells. Moreover, Atf6a-deleted mice displayed hepatic dysfunction and steatosis much longer than wild-type mice in response to pharmacological induction of ER stress. This could be explained by chronic expression of CHOP and sustained suppression of C/EBPa and/or a failure of ATF6a-mediated induction of genes encoding protein chaperone, trafficking, and ERAD functions. When fed a HFD, Atf6a−/− mice developed hepatic steatosis and glucose intolerance in association with increased expression of SREBP-lc. On the other hand, overexpression of a functionally active nuclear fragment of ATF6 in zebrafish caused fatty liver, suggesting that fine-tuning of ATF6a may be important to prevent liver steatosis.

In some embodiments, a compound or salt thereof described herein or a composition described herein may be used in a method of treating metabolic disorders, such as obesity, type I- and type II diabetes, pancreatitis, dyslipidemia, hyperlipidemia conditions, non-alcoholic fatty liver disease (NAFLD), non-alcoholic steatohepatitis (NASH), insulin resistance, hyperinsulinemia, glucose intolerance, hyperglycemia, metabolic syndrome, acute myocardial infarction, hypertension, cardiovascular diseases, atherosclerosis, peripheral arterial disease, apoplexy, heart failure, coronary artery heart disease, renal disease, diabetic complications, neuropathy, gastroparesis, disorder associated with a serious inactivation mutation in insulin receptor, and other metabolic disorders.

### Compositions

Provided herein are pharmaceutical compositions including any of the ATFi's (e.g., synthetic ATFi's) described herein (e.g., ATFi's configured to target an ATF, as described herein) in a pharmaceutically acceptable carrier. The pharmaceutical compositions described herein are substantially devoid of contaminates, such viral particles, viral capsid proteins, or peptide fragments thereof. In some embodiments, the pharmaceutical compositions provided herein are non-immunogenic. For example, non-immunogenic pharmaceutical compositions may be substantially devoid of pathogen-associated molecular patterns recognizable by cells of the innate immune system. Such pathogen-associated molecular patterns include CpG motifs (e.g., unmethylated CpG motifs or hypomethylated CpG motifs), endotoxins (e.g., lipopolysaccharides (LPS), e.g., bacterial LPS), flagellin, lipoteichoic acid, peptidoglycan, and viral nucleic acids molecules, such as double-stranded RNA. In some embodiments, the pharmaceutical compositions provided herein comprise a compound of Formulas (I)-(III), or a pharmaceutically acceptable salt thereof, in a pharmaceutically acceptable carrier. In some embodiments, the pharmaceutical compositions provided herein comprise a compound of Table 1, 2 or 3, or a pharmaceutically acceptable salt thereof, in a pharmaceutically acceptable carrier.

In some embodiments, a pharmaceutical composition is administered to a subject in need thereof. The pharmaceutical composition contains a therapeutically effective amount of a compound. In some embodiments, the compound comprises a synthetic activating transcription factor inhibitor. In some embodiments, the synthetic activating transcription factor inhibitor includes a synthetic antisense oligonucleotide or a small molecule inhibitor. In certain aspects, a pharmaceutical composition comprises a compound, or a pharmaceutically acceptable salt thereof. In some embodiments, the compound modulates an ATF. In some embodiments, the ATF is an ATF6. In some embodiments the pharmaceutical composition comprises a second agent. The second agent modulates the unfolded protein response (UPR) or the integrated stress response (ISR) in a cell environment. in some embodiments, the second agent modulates a pathway. In some embodiments, the pathway is a IRE1/XBP1 pathway.

In reference to hepatic conditions or other unwanted hepatic diseases, beneficial or desired results include reducing the levels of a targeted protein; decreasing the proliferation of disease-related proteins (such as to suppress production of the protein via modulation of a cellular pathway); reducing the number of proteins associated with a hepatic condition; inhibiting or slowing to some extent and preferably stopping liver cells from producing or expressing the protein associated with a hepatic condition. In some embodiments, the hepatic condition comprises non-alcoholic fatty liver disease (NAFLD), non-alcoholic steatohepatitis (NASH), hepatocyte ballooning, hepatocyte cell death and fibrosis, or cirrhosis and hepatocellular carcinoma.

The pharmaceutical compositions described herein may be assessed for contamination by conventional methods and formulated into a pharmaceutical composition intended for a suitable route of administration. Still, other compositions containing the ATFi may be formulated similarly with a suitable carrier. Such formulation involves the use of a pharmaceutically and/or physiologically acceptable vehicle or carrier, particularly directed for administration to the target cell. In one embodiment, carriers suitable for administration to the target cells include buffered saline, an isotonic sodium chloride solution, or other buffers, e.g., EDTA or HEPES, to maintain pH at appropriate physiological levels, and, optionally, other medicinal agents, pharmaceutical agents, stabilizing agents, buffers, carriers, adjuvants, or diluents. In some embodiments, the carrier is a liquid for injection. Exemplary physiologically acceptable carriers include sterile, pyrogen-free water and sterile, pyrogen-free, phosphate buffered saline. In one embodiment, the carrier is an isotonic sodium chloride solution. In another embodiment, the carrier is balanced salt solution. In one embodiment, the carrier includes tween. If the vector is to be stored long-term, it may be frozen in the presence of glycerol or Tween20.

A “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley—VCH 2002. S. M. Berge, L. D. Bighley, D. C. Monkhouse, J. Pharm. Sci. 1977,66, 1-19. P. H. Stahl and C. G. Wermuth, editors, Handbook of Pharmaceutical Salts: Properties, Selection and Use, Weinheim/Zurich:Wiley-VCH/VHCA, 2002, each of which is incorporated by reference in its entirety. In some embodiments, the therapeutically active agent is a compound of Formulas (I)-(III). In some embodiments, the therapeutically active agent is a compound of Table 1, 2 or 3.

In other embodiments, compositions containing vectors described herein include a surfactant. Useful surfactants, such as Pluronic F68 (Poloxamer 188, also known as LUTROL® F68) may be included as they prevent some carriers from sticking to inert surfaces and thus ensure delivery of the desired dose.

Delivery vehicles such as nucleic acid vectors, liposomes, nanoparticles, microparticles, microspheres, lipid particles, vesicles, and the like, may be used for the introduction of the compositions of the present disclosure into suitable host cells. In particular, DNA vectors may be formulated for delivery by encapsulation in a lipid particle, a liposome, a vesicle, or a nanoparticle. In some embodiments, the DNA vector is complexed with a delivery vehicle such as a poloxamer and/or polycationic material. In some cases, the nucleic acid vector is a viral vector. In some embodiments, the vector is a plasmid vector. In some embodiments, the nucleic acid vector is circular. In some embodiments the nucleic acid vector is linear.

### Carriers and Excipients

The compositions disclosed herein are formulated in any suitable manner for administration. Any suitable technique, carrier, and/or excipient is contemplated for use with the compositions disclosed herein. Non-limiting examples of cosmetic, dermatological, or pharmaceutically acceptable carriers and excipients suitable for formulation can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; Pharmaceutical Dosage Forms and Drug Delivery Systems, Eighth Ed. (Lippincott Williams & Wilkins 2004); and Muller, R. H., et al., Advanced Drug Delivery Reviews 59 (2007) 522-530, each of which is incorporated by reference in its entirety.

In some embodiments, the pharmaceutically acceptable carriers or excipients disclosed herein include, but are not limited to one or more: pH modifying agent (e.g., buffering agents), stabilizing agents, thickening agents, colorant agents, preservative agents, emulsifying agents, solubilizing agents, antioxidant agents, or any combination thereof. Other suitable compounds contemplated herein and within the knowledge of a practitioner skilled in the relevant art are found in the Handbook of Pharmaceutical Excipients, 4th Ed. (2003), the entire content of which is incorporated by reference herein.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or salt thereof, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cata-plasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein, or a salt thereof, can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20th ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shells can be plant oils, wax, fats, semi solid or liquid polyols.

Any compounds disclosed herein can be formulated in a tablet in any dosage form described, such as a compound described herein, or a pharmaceutically acceptable salt thereof, can be formulated as a tablet.

In some embodiments, the carrier may be a native, labeled or synthetic protein, a synthetic chemical polymer of precisely known chemical composition or with a distribution around a mean molecular weight, an oligonucleotide, a phosphorodiamidate morpholino oligomer (PMO), or a foldamer, a lipid, a lipid micelle, or a nanoparticle. In some embodiments, the carrier is a liposome. In some embodiments, the carrier is a lipid particle. In some embodiments, the carrier is a vesicle. In some embodiments, the carrier is a polyaxamer or polycationic material.

In some embodiments, the carrier may be a polyethylene glycol (PEG). PEG has been studied comprehensively as a carrier because it is soluble in both organic and hydrophilic solvents. Unlike many other synthetic polymers, PEG is relatively hydrophilic. Conjugation with PEG increases the solubility of hydrophobic molecules and prolongs the circulation time in the organism. PEG also minimizes the nonspecific absorption of a molecule, such as a drug, provides specific affinity toward the targeted tissue, and increases the drug accumulation in malignant tissue. PEG can be conjugated to other polymers to make them less hydrophobic (i.e., PEGylation). The changes in surface hydrophilicity prevent protein adsorption, thereby enabling cell adhesion and proliferation on biomaterial scaffolds. The PMO backbone is made of morpholino rings with phosphorodiamidate linkage, which protects them from nuclease degradation while still maintaining the complementary base pairing. The potential application of PMO-based antisense technology targeting bacterial pathogens is being explored for the development of a new class of antibacterial drugs. Panchal R G, Peptide Conjugated Phosphorodiamidate Morpholino Oligomers Increase Survival of Mice Challenged with Ames Bacillus anthracis., Nucleic Acid Ther., 2012; 22(5): 316-322.

In some embodiments, the compositions disclosed herein comprise one or more preservatives. The preservative, when utilized, is in an amount sufficient to extend the shelf-life or storage stability, or both, of the topical formulations disclosed herein. Exemplary preservatives include, but are not limited to, tetrasodium ethylene-diamine tetraacetic acid (EDTA), methyl, ethyl, butyl, and propyl parabens, benzophenone-4, methylchloroisothiazolinone, methylisothiazolinone, sodium benzoate, paraoxybenzoic acid esters, chlorobutanol, benzyl alcohol, phenylethylalcohol, dehydroacetic acid, sorbic acid, benzalkonium chloride (BKC), benzethonium chloride, phenol, phenylmercuric nitrate, and thimerosal.

### Methods of Use

In some embodiments, provided herein is a method of inhibiting the ATF6 pathway. In some embodiments, provided herein is a method of inhibiting the ATF6. In some embodiments, the ATF6 is ATF6a. The compounds or salts thereof described herein, and compositions described herein, are believed to be effective for inhibiting the ATF6 pathway, ATF6, and/or ATF6a.

In some embodiments, the method of inhibiting the ATF6 pathway, ATF6, or ATF6a comprises administering or delivering to a cell comprising ATF6 or ATF6a a compound described herein, or a pharmaceutically acceptable salt thereof, or a pharmaceutical composition described herein. In some embodiments, the cell is a diseased cell, such as a cancer cell. In some embodiments, the cell has an activated ATF6 pathway. In some embodiments, the cell has been exposed to an ER stress-inducing condition. Several ER stress-inducing conditions are known in the art, such as glucose deprivation, aberrant Ca2+ regulation, viral infection, hypoxia, and exposure to an ER stress-inducing molecule such as thapsigargin, ionomycin, or tunicamycin.

In some embodiments, the method of inhibiting the ATF6 pathway, ATF6, or ATF6a comprises administering or delivering a compound described herein, or a pharmaceutically acceptable salt thereof, or a pharmaceutical composition described herein to a tumor.

In some embodiments, the inhibition of the ATF6 pathway, ATF6, or ATF6a comprises inhibiting expression of an ATF6 and/or ATF6a target gene. In some embodiments, the inhibition of the ATF6 pathway, ATF6, or ATF6a comprises inhibiting expression of an ATF6a target gene. In some embodiments, the expression of the ATF6 and/or ATF6a target gene is inhibited by at least about 10%, 20%, 30%, 40%, 50%, 60%, 70%, 80%, 90%, 95%, or 98%.

In some embodiments, the ATF6 and/or ATF6a target gene comprises a promoter comprising a ER-stress responsive element (ERSE). In some embodiments, the promoter comprises a sequence that shares at least about 70%, 75%, 80%, 85%, 90%, 95%, 96%, 97%, 98%, or 99% sequence identity with CCAATCGGCGGCGGCCACG (SEQ ID NO. 1). In some embodiments, the promoter comprises SEQ ID NO. 1. In some embodiments, the ATF6 and/or ATF6a target gene is GRP78, HERPUD1, or ERO1B. In some embodiments, the ATF6a target gene is GRP78. Inhibition of expression of an ATF6 and/or ATF6a target gene can be determined by methods known in the art, such as by detection of the mRNA of the target gene using techniques such as PCR, qPCR, or northern blotting, or by detection of polypeptide gene product, such as by immunoblotting, western blotting, or mass spectrometry.

In some embodiments, the compound, salt thereof, or composition inhibits the ATF6 pathway, ATF6, or ATF6a with an IC50 of less than about 10 μM, such as less than about 5 μM, 2 μM, 1 μM, 900 nM, 800 nM, 700 nM, or 600 nM. In some embodiments, the compound, salt thereof, or composition inhibits the ATF6 pathway, ATF6, or ATF6a with an IC50 between about 10 nM and 5 μM, such between about 50 nM and 2 μM, 100 nM and 1 μM, or 20 nM and 1 μM. The half maximal inhibitory concentration (IC50) is a measure of the effectiveness of a substance in inhibiting a specific biological or biochemical function. The IC50 is a quantitative measure that indicates how much of an inhibitor is needed to inhibit a given biological process or component of a process such as an enzyme, cell, cell receptor or microorganism by half.

Methods of determining IC50 in vitro and in vivo are known in the art.

In some embodiments, the compounds or salts thereof described herein, and compositions described herein, are administered in an amount wherein ATF6P activity is not inhibited or is inhibited to a lesser extent. In some embodiments, inhibition of ATF6a is at least or at least about 2-fold greater than inhibition of ATF6P activity, for example at least or at least about 3 fold, 4 fold, 5 fold, 8 fold, 10 fold, 15 fold, 30 fold, 50 fold, 60 fold, 75 fold, or 100 fold greater.

Provided herein is a method of treating a disease in an individual comprising administering an effective amount of a compound of Formulas (I)-(III) or any embodiment, variation or aspect thereof, including any variation or aspect thereof, (collectively, a compound of Formulas (I)-(III) or the present compounds or the compounds detailed or described herein) or a pharmaceutically acceptable salt thereof, to the individual.

In some embodiments, provided herein is a method of treating a disease mediated by the ATF6 pathway in an individual comprising administering an effective amount of a compound of Formulas (I)-(III), or a pharmaceutically acceptable salt thereof, to the individual. In some embodiments, provided herein is a method of treating a disease mediated by the activation of the ATF6 pathway in an individual comprising administering an effective amount of a compound of Formulas (I)-(III), or a pharmaceutically acceptable salt thereof, to the individual. In some embodiments, provided herein is a method of treating a disease mediated by the activation of ATF6 in an individual comprising administering an effective amount of a compound of Formulas (I)-(III), or a pharmaceutically acceptable salt thereof, to the individual. In some embodiments, provided herein is a method of treating a disease mediated by the activation of ATF6a in an individual comprising administering an effective amount of a compound of Formulas (I)-(III), or a pharmaceutically acceptable salt thereof, to the individual.

In some embodiments, provided herein is a method of treating a disease characterized by activation of the ATF6 pathway in an individual comprising administering an effective amount of a compound of Formulas (I)-(III), or a pharmaceutically acceptable salt thereof, to the individual. In some embodiments, provided herein is a method of treating a disease characterized by activation of ATF6 in an individual comprising administering an effective amount of a compound of Formulas (I)-(III), or a pharmaceutically acceptable salt thereof, to the individual. In some embodiments, provided herein is a method of treating a disease characterized by activation of ATF6a in an individual comprising administering an effective amount of a compound of Formulas (I)-(III), or a pharmaceutically acceptable salt thereof, to the individual. In some embodiments, provided herein is a method of treating a disease characterized by increased expression of an ATF6 target gene in an individual comprising administering an effective amount of a compound of Formulas (I)-(III), or a pharmaceutically acceptable salt thereof, to the individual. In some embodiments, provided herein is a method of treating a disease characterized by increased expression of an ATF6a target gene in an individual comprising administering an effective amount of a compound of Formulas (I)-(III), or a pharmaceutically acceptable salt thereof, to the individual. In some embodiments, the increased expression is in comparison to a non-diseased tissue or cell.

The present compounds or salts thereof are believed to be effective for treating a variety of diseases and disorders, such as diseases wherein ATF6-activated transcription targets play a role in the pathogenisis or development of the disease. For example, in some embodiments, the present compounds and compositions may be used to treat viral infection, cancer, a neurodegenerative disease, or a vascular disease, such as a cardiovascular disease. In some embodiments, the disease is viral infection, hereditary cerebellar atrophy and ataxia, or Alzheimer's disease. In some embodiments, the disease is type 2 diabetes mellitus or diabetic nephropathy. In some embodiments, the disease is myocardial atrophy, heart failure, atherosclerosis, or ischemia, such as ischemic heart disease or cerebral ischemia.

It has been demonstrated that ATF6 branch of the UPR is central for viral infection. For example, ATF6 is important for maintaining cell viability and modulating immune responses during West Nile virus infection (Ambrose R J, Virol., 2013 February; 87(4): 2206-2214). Also, African swine fever virus activates ATF6 branch to prevent early apoptosis and ensure viral replication (Galindo I, Cell Death Dis, 2012 July; 3(7): e341). Accordingly, in some embodiments, a compound or salt thereof described herein or a composition described herein may be used in a method of treating or preventing a viral infection. In some embodiments, the viral infection is an African swine fever virus, a dengue virus, an enterovirus, a hepatitis B virus, a hepatitis C virus, influenza virus, a tick-borne encephalitis virus, or a WestNile virus infection. In some embodiments, the viral infection is caused by a virus that activates ATF6 in an infected cell.

In some embodiments, a compound or salt thereof described herein or a composition described herein may be used in a method of treating cancer, such as liver cancer.

The capacity of the UPR signaling arms to distinctly influence ER proteostasis and function suggests that selective activation of these pathways has significant potential to alleviate pathologic imbalances in ER proteostasis associated with etiologically diverse human diseases. In particular, activation of the ATF6 signaling arm has been shown to be useful for ameliorating disease-associated imbalances in ER proteostasis and function. The stress-independent activation of the ATF6 transcription factor using a chemical genetic approach induces protective remodeling of ER proteostasis pathways to selectively reduce secretion and extracellular aggregation of destabilized, amyloid disease-associated proteins, such as transthyretin and immunoglobulin light chain, without significantly impacting the secretion of the endogenous proteome. Accordingly, a compound or salt thereof described herein or a composition described herein may be used in a method for correcting pathologic imbalances in ER proteostasis in cellular and animal models of protein misfolding and aggregation diseases.

### Embodiments

Embodiment 1 comprises a method of treating a subject having a hepatic condition, the method comprising administering a pharmaceutical composition comprising a therapeutically effective amount of a synthetic activating transcription factor inhibitor (ATFi), wherein the synthetic ATFi modulates a signaling of an activating transcription factor (ATF) in a cell via interference with a cellular pathway.

Embodiment 2 comprises the method of embodiment 1, wherein the interference with the cellular pathway comprises sequestering an ATF protein as a transmembrane protein in an endoplasmic reticulum, preventing trafficking of the ATF protein from the endoplasmic reticulum to a Golgi apparatus, cleaving the ATF protein via one or more proteases, or blocking trafficking of a soluble and active ATF fragment to a nucleus of the cell, or a combination thereof.

Embodiment 3 comprises the method of embodiment 1 or embodiment 2, wherein the synthetic ATFi is a synthetic antisense oligonucleotide.

Embodiment 4 comprises the method of embodiment 3, wherein the synthetic antisense oligonucleotide knocks down a nucleic acid transcript, thereby reducing levels of a targeted protein via binding to a target mRNA.

Embodiment 5 comprises the method of embodiment 3 or embodiment 4, wherein the synthetic antisense oligonucleotide is conjugated to a triantennary N-acetyl galactosamine (GalNAc).

Embodiment 6 comprises the method of embodiment 5, wherein the GalNAc targets asialoglycoprotein receptors.

Embodiment 7 comprises the method of any one of embodiments 1-6, wherein the cell comprises a hepatocyte.

Embodiment 8 comprises the method of any one of embodiments 1-7, wherein the hepatic condition is non-alcoholic fatty liver disease (NAFLD), non-alcoholic steatohepatitis (NASH), hepatocyte ballooning, hepatocyte cell death and fibrosis, or cirrhosis and hepatocellular carcinoma.

Embodiment 9 comprises the method of any one of embodiments 1-8, wherein the pharmaceutical composition is administered to the subject via subcutaneous injection, intramuscular injection, or intravenous injection.

Embodiment 10 comprises the method of any one of embodiments 1-9, wherein the ATF is ATF6.

Embodiment 11 comprises a method of detecting a hepatic condition in a subject, the method comprising: administering a therapeutically effective amount of a pharmaceutical composition to the subject suspected of having the hepatic condition, wherein the pharmaceutical composition comprises an activating transcription factor inhibitor (ATFi) configured to modulate an activating transcription factor (ATF); determining an efficacy of the ATFi via comparing a first level of the ATF before administering the ATFi to the subject and a second level of the ATF after administering the ATFi to the subject; and measuring a biomarker level associated with the hepatic condition after administering the ATFi to the subject, thereby detecting a presence or absence of the hepatic condition.

Embodiment 12 comprises the method of embodiment 11, wherein the biomarker is screened via digital spatial profiling.

Embodiment 13 comprises the method of embodiment 11 or embodiment 12, wherein the ATFi is an antisense oligonucleotide.

Embodiment 14 comprises the method of embodiment 11 or embodiment 12, wherein the ATFi is a small-molecule inhibitor.

Embodiment 15 comprises the method of embodiment 14, wherein the small molecule inhibitor is Ceapin A7.

Embodiment 16 comprises the method of embodiment 14, wherein the small molecule inhibitor comprises a compound of Table 1, Table 2, or Table 3.

Embodiment 17 comprises the method of any one of embodiments 11-16, wherein determining the efficacy of the ATFi is performed in vitro.

Embodiment 18 comprises the method of any one of embodiments 11-16, wherein the determining the efficacy of the ATFi is performed in vivo.

Embodiment 19 comprises the method of any one of embodiments 11-18, wherein the hepatic condition is non-alcoholic fatty liver disease (NAFLD), non-alcoholic steatohepatitis (NASH), hepatocyte ballooning, hepatocyte cell death and fibrosis, cirrhosis, hepatocellular carcinoma, cholangicarcinoma, metastatic liver cancer, or any combination thereof.

Embodiment 20 comprises the method of any one of embodiments 11-19, wherein the pharmaceutical composition is administered to the subject via oral administration, subcutaneous injection, intramuscular injection, or intravenous injection.

Embodiment 21 comprises the method of any one of embodiments 11-20, wherein the ATF is ATF6.

Embodiment 22 comprises a method of inhibiting expression of an activating transcription factor (ATF) gene in a cell of a subject, the method comprising administering to the subject a therapeutically effective amount of a pharmaceutical composition comprising a synthetic activating transcription factor inhibitor (ATFi) and a suitable delivery vehicle.

Embodiment 23 comprises the method of embodiment 22, wherein the synthetic ATFi is a synthetic antisense oligonucleotide.

Embodiment 24 comprises the method of embodiment 23, wherein the synthetic antisense oligonucleotide knocks down a gene encoding the ATF via binding to a target mRNA.

Embodiment 25 comprises the method of any one of embodiments 22-24, wherein the ATF is ATF6.

Embodiment 26 comprises the method of any one of embodiments 23-25, wherein the synthetic antisense oligonucleotide is conjugated to a triantennary N-acetyl galactosamine (GalNAc).

Embodiment 27 comprises the method of embodiment 26, wherein the GalNAc targets asialoglycoprotein receptors.

Embodiment 28 comprises the method of any one of embodiments 22-27, wherein the cell is a hepatocyte.

Embodiment 29 comprises the method of embodiment 28, wherein the subject has a hepatic condition.

Embodiment 30 comprises the method of embodiment 28, wherein the hepatic condition is non-alcoholic fatty liver disease (NAFLD), non-alcoholic steatohepatitis (NASH), hepatocyte ballooning, hepatocyte cell death and fibrosis, cirrhosis, or liver cancer, or any combination thereof.

Embodiment 31 comprises the method of any one of embodiments 22-30, wherein the pharmaceutical composition is administered to the subject via subcutaneous injection, intramuscular injection, or intravenous injection.

Embodiment 32 comprises the method of any one of embodiments 22-31, wherein the pharmaceutical composition further comprises a suitable delivery vehicle comprising liposomes, nanoparticles, microparticles, microspheres, lipid particles, vesicles, polyaxamer, or polycationic material.

Embodiment 33 comprises a method of inhibiting an activating transcription factor (ATF) in a cell of a subject, the method comprising: administering to the subject a therapeutically effective amount of a pharmaceutical composition comprising a synthetic activating transcription factor inhibitor (ATFi) and a suitable delivery vehicle, wherein the synthetic ATFi, following administration to the subject, couples to a receptor in the cell, thereby inhibiting the ATF in the cell.

Embodiment 34 comprises the method of embodiment 33, wherein the synthetic ATFi is a synthetic antisense oligonucleotide.

Embodiment 35 comprises the method of embodiment 34, wherein the synthetic antisense oligonucleotide knocks down an endonuclease transcript and reduces levels of a targeted protein via binding to a target mRNA.

Embodiment 36 comprises the method of embodiment 35, wherein the targeted protein is an ATF6 protein.

Embodiment 37 comprises the method of any one of embodiments 34-36, wherein the synthetic antisense oligonucleotide is conjugated to a triantennary N-acetyl galactosamine (GalNAc).

Embodiment 38 comprises the method of embodiment 37, wherein the GalNAc targets asialoglycoprotein receptors.

Embodiment 39 comprises the method of any one of embodiments 33-38, wherein the cell is a hepatocyte.

Embodiment 40 comprises the method of any one of embodiments 33-39, wherein the subject has a hepatic condition.

Embodiment 41 comprises the method of embodiment 40, wherein the hepatic condition is non-alcoholic fatty liver disease (NAFLD), non-alcoholic steatohepatitis (NASH), hepatocyte ballooning, hepatocyte cell death and fibrosis, cirrhosis, or hepatocellular carcinoma.

Embodiment 42 comprises the method of any one of embodiments 33-41, wherein the pharmaceutical composition is administered to the subject via subcutaneous injection, intramuscular injection, or intravenous injection.

Embodiment 43 comprises the method of any one of embodiments 33-42, wherein the suitable delivery vehicle is selected from the group consisting essentially of liposomes, nanoparticles, microparticles, microspheres, lipid particles, vesicles, polyaxamer, native, labeled or synthetic proteins, and polycationic materials.

Embodiment 44 comprises the method of any one of embodiments 1-43, wherein the subject is a human.

Embodiment 45 comprises the method of any one of embodiments 1-44, wherein the subject is over 30, 40, 50, 60, 70 or 80 years of age, or an age within a range defined by of any of the preceding numbers.

Embodiment 46 comprises the method of any one of embodiments 1, 2, 7-12, 17-22, 28-33 and 39-45, wherein the ATFi is a small-molecule inhibitor.

Embodiment 47 comprises the method of embodiment 46, wherein the small molecule inhibitor is Ceapin A7, or a pharmaceutically acceptable salt thereof.

Embodiment 48 comprises method of embodiment 46, wherein the small molecule inhibitor comprises a compound of Table 1, Table 2, or Table 3, or a pharmaceutically acceptable salt thereof.

Embodiment 49 comprises the method of any one of embodiments 3-6, 13, 23-27 and 34-38, further comprising administering to the subject a pharmaceutical composition comprising a synthetic activating transcription factor inhibitor (ATFi) and a suitable delivery vehicle, wherein the ATFi is a small-molecule inhibitor.

Embodiment 50 comprises the method of embodiment 49, wherein the small molecule inhibitor is Ceapin A7, or a pharmaceutically acceptable salt thereof.

Embodiment 51 comprises the method of embodiment 49, wherein the small molecule inhibitor comprises a compound of Table 1, Table 2, or Table 3, or a pharmaceutically acceptable salt thereof.

Embodiment 52 comprises the method of embodiment 51, wherein the compound of Table 1, Table 2, or Table 3, or a pharmaceutically acceptable salt thereof, has at least one hydrogen atom with an isotopic distribution in proportional amounts different to those usually found in nature at any position that a hydrogen atom is present.

Embodiment 53 comprises the method of embodiment 52, wherein the compound of Table 1, Table 2, or Table 3, or a pharmaceutically acceptable salt thereof, has one, two, three, four, five or six hydrogen atoms with deuterium in proportional amounts greater than usually found in nature.

Embodiment 54 comprises the method of embodiment 53, wherein each of the one, two, three, four, five or six hydrogen atoms independently includes at least 52.5% deuterium incorporation, at least 60% deuterium incorporation, at least 67.5% deuterium incorporation, at least 75% deuterium incorporation, at least 82.5% deuterium incorporation, at least 90% deuterium incorporation, at least 95% deuterium incorporation, at least 97% deuterium incorporation, at least 99% deuterium incorporation, or at least 99.5% deuterium incorporation.

Embodiment 55 comprises the method of embodiment 54, wherein each of the one, two, three, four, five or six hydrogen atoms includes less than 99.9% deuterium incorporation.

### Examples

The following examples are illustrative and non-limiting to the scope of the compositions, methods, and formulations described herein.

### Example 1: The UPR Transducer ATF6 Drives Fatty Liver Disease Progression to Carcinoma

A quarter of the world's population suffers from non-alcoholic fatty liver disease (NAFLD) characterized by abnormal lipid accumulation (steatosis) that can progress to steatohepatitis (NASH) with inflammation, hepatocyte ballooning, cell death and fibrosis, cirrhosis and hepatocellular carcinoma (HCC), the most common primary liver cancer and a leading cause of cancer-related deaths. NAFLD has been linked with endoplasmic reticulum (ER) stress and activation of the unfolded protein response (UPR). Activating transcription factor 6 (ATF6) is required to survive acute ER stress and resolve steatosis in hepatocytes, although the role of ATF6 in NASH-related HCC has not been examined. Chronic and aberrant ATF6 activation drives NAFLD progression to NASH and HCC through ER stress, UPR induction and a metabolic shift in hepatocytes favoring cancer development.

Cancer databases were explored for Atf6 mRNA expression and ATF6 protein expression was characterized by immunohistochemistry of liver tissue from normal, NAFLD, NASH and HCC patients. Mice with activated ATF6 (TG-Atf6p50^{Hep}) or Atf6 deletion were used, notably in dietary, oncogenic and MUP-uPA murine models of chronic liver disease.

ATF6 activation is increased in human NASH/HCC. Patient liver IHC revealed increasingly cytoplasmic to nuclear ATF6 expression as chronic liver disease progresses to HCC, implying activation of ATF6-dependent transcriptional signaling. In mice, 2-week hepatocyte-specific ATF6p50 induction causes hepatocellular damage, upregulation of NRF2-induced genes, and accumulation of lipids/cholesterol in the liver. Chronic and aberrant ATF6p50 expression promotes diethylnitrosamine (DEN)+HFD-induced liver cancer, with all mice developing well-differentiated HCC, while WT mice developed no tumoral lesions, adenomas or early HCC nodules. Tumor compared to non-tumor tissues have less ATF6p90 and more ATF6p50 and its target ER chaperones (BiP, GRP94), associated with adaptive UPR and pro-survival signaling. In contrast, CHOP is increased in non-tumoral TG-Atf6p50^{He}P liver tissue compared to WT, potentially promoting cell death and compensatory proliferation in a NAFLD/NASH environment. In contrast, Atf6-deletion attenuates NASH/HCC development after DEN+HFD or western diet feeding. Hepatocyte-specific Atf6 deletion protects from oncogene-induced HCC. Finally, livers from Atf6-deleted mice exhibit downregulated NRF2-induced mRNAs, notably after HFD challenge. Consistent with this notion, FBP1 protein and mRNA are reduced in TG-Atf6p50^{Hep} mice, and to a greater degree in tumor vs. nontumor tissue, possibly stabilizing NRF2, a bonafide HCC oncoprotein. Liver ATF6 ChIP-Seq analysis demonstrated ATF6 binds the promoter regions of Nfe212 (NRF2) and cholesterol biosynthetic genes. The newly discovered oncogenic factor ATF6, a key UPR regulator of ER stress, protein homeostasis and cholesterol metabolism, is implicated in NASH-driven HCC upon aberrant activation, which should lead to new biomarkers for HCC progression and therapeutic strategies for intervention.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

### Example 2. Transgenic ATF6p50^{Hep}/MUP Mice Models

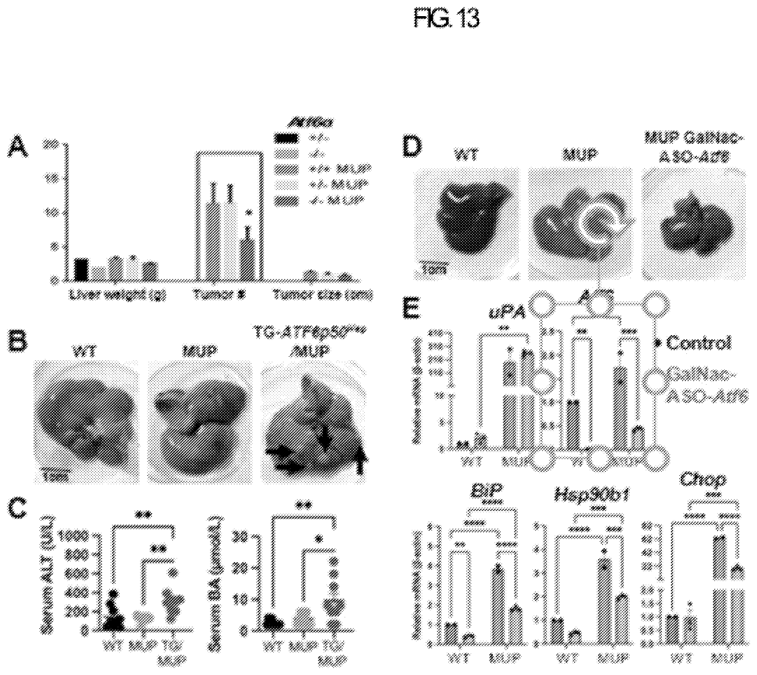

Studies indicate that Atf6 deletion attenuates HCC in MUP-uPA* mice, which develop NASH-driven HCC due to transient ER stress and subsequent high-fat diet (HFD) feeding, as depicted in FIG. 13A. When MUP-uPA mice were crossed with TG-Atf6p50 mice, 24 wks of HFD feeding was sufficient to induce early HCC nodules and a significant increase in serum ALT and BA levels, in contrast to WT or MUP-uPA mice, as reflected in FIGS. 13B-C. In some example embodiments, Atf6 knockdown is performed in vivo using a non-toxic therapeutic strategy, administering GalNac-conjugated ASO targeting Atf6 (GalNac-ASO-Atf6) to MUP-uPA mice. GalNac-ASO-Atf6 treatment in WT mice had no phenotype, other than reduced hepatocyte Atf6 (97%) and BiP (55%) mRNAs. In contrast, treating young MUP-uPA mice led to a 74% knockdown in Atf6 mRNA, causing a significant decrease in UPR transcripts without affecting uPA levels, as depicted in FIGS. 13D-E.

In other example embodiments, a model of NASH-HCC (transgenic UP-uPA mice), where urokinase plasminogen activator (uPA) that is overexpressed in hepatocytes misfolds, caused hepatocyte ER stress at 4-6 wks of age that is attenuated after 10 wks due to extinction of uPA transgene expression (Nakagawa H, ER stress cooperates with hypernutrition to trigger TNF-dependent spontaneous HCC development, Cancer Cell, 2014, 26(3): 331-343). When fed a HFD at 10 wks of age, these mice develop symptoms of human NASH and by 9mon develop HCC. Thus, transient ER stress is sufficient to establish an environment conducive for NASH and HCC development upon subsequent HFD feeding.

### Example 3. Synthetic Compounds

The compounds in Tables 1-3, can be synthesized by one of skill in the art using synthetic procedures as shown in US20190367497A1, WO2021069701A1 and WO2021069721A1, each of which is incorporated by reference in its entirety. Compounds described herein for use in treating a hepatic condition can be found in Table 1-3.

TABLE 1
Compounds 1-1 to 1-26

| Compound # | Compound Structure |
|---|---|
| 1-1 | 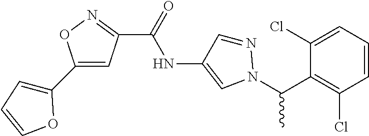 [SMILES: CC(c1c(Cl)cccc1Cl)n1cc(NC(=O)c2cc(-c3ccco3)on2)cn1] |
| 1-2 | 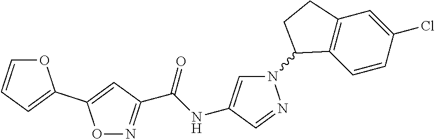 [SMILES: O=C(Nc1cnn(C2CCc3cc(Cl)ccc32)c1)c1cc(-c2ccco2)on1] |
| 1-3 | 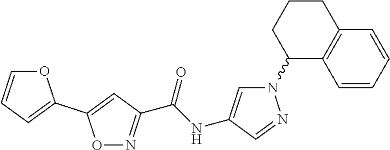 [SMILES: O=C(Nc1cnn(C2CCCc3ccccc32)c1)c1cc(-c2ccco2)on1] |
| 1-4 | 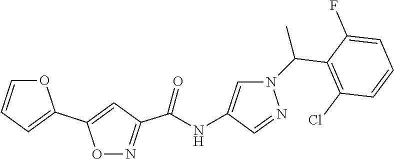 [SMILES: CC(c1c(F)cccc1Cl)n1cc(NC(=O)c2cc(-c3ccco3)on2)cn1] |
| 1-5 | 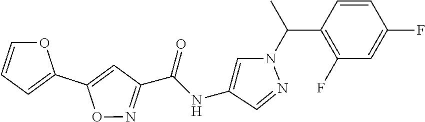 [SMILES: CC(c1ccc(F)cc1F)n1cc(NC(=O)c2cc(-c3ccco3)on2)cn1] |
| 1-6 | 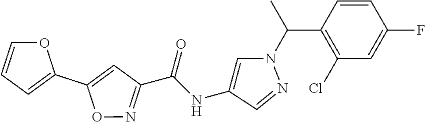 [SMILES: CC(c1ccc(F)cc1Cl)n1cc(NC(=O)c2cc(-c3ccco3)on2)cn1] |
| 1-7 | 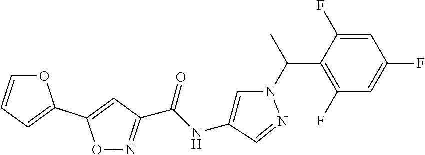 [SMILES: CC(c1c(F)cc(F)cc1F)n1cc(NC(=O)c2cc(-c3ccco3)on2)cn1] |
| 1-8 | 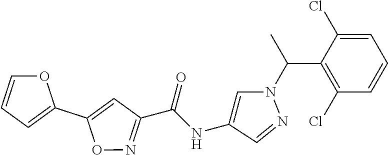 [SMILES: CC(c1c(Cl)cccc1Cl)n1cc(NC(=O)c2cc(-c3ccco3)on2)cn1] |
| 1-9 | 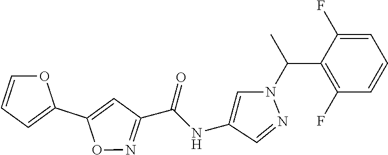 [SMILES: CC(c1c(F)cccc1F)n1cc(NC(=O)c2cc(-c3ccco3)on2)cn1] |
| 1-10 | 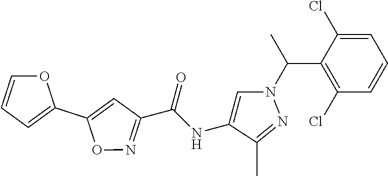 [SMILES: Cc1nn(C(C)c2c(Cl)cccc2Cl)cc1NC(=O)c1cc(-c2ccco2)on1] |
| 1-11 | 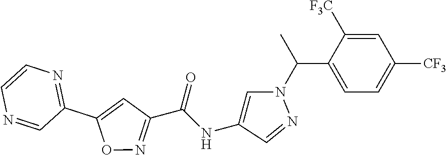 [SMILES: CC(c1ccc(C(F)(F)F)cc1C(F)(F)F)n1cc(NC(=O)c2cc(-c3cnccn3)on2)cn1] |
| 1-12 | 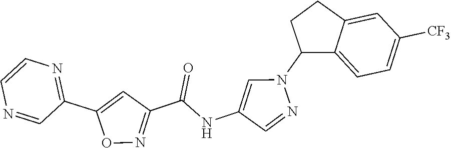 [SMILES: O=C(Nc1cnn(C2CCc3cc(C(F)(F)F)ccc32)c1)c1cc(-c2cnccn2)on1] |
| 1-13 | 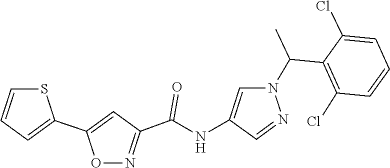 [SMILES: CC(c1c(Cl)cccc1Cl)n1cc(NC(=O)c2cc(-c3cccs3)on2)cn1] |
| 1-14 | 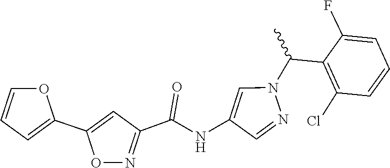 [SMILES: CC(c1c(F)cccc1Cl)n1cc(NC(=O)c2cc(-c3ccco3)on2)cn1] |
| 1-15 | 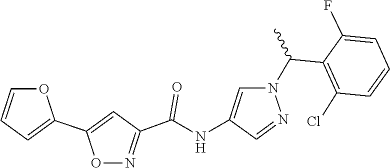 [SMILES: CC(c1c(F)cccc1Cl)n1cc(NC(=O)c2cc(-c3ccco3)on2)cn1] |
| 1-16 | 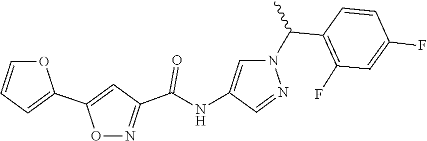 [SMILES: CC(c1ccc(F)cc1F)n1cc(NC(=O)c2cc(-c3ccco3)on2)cn1] |
| 1-17 | 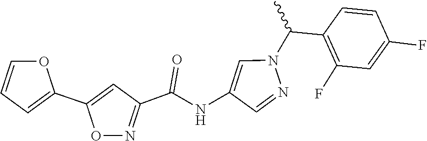 [SMILES: CC(c1ccc(F)cc1F)n1cc(NC(=O)c2cc(-c3ccco3)on2)cn1] |
| 1-18 | 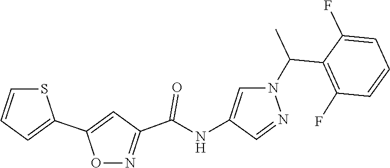 [SMILES: CC(c1c(F)cccc1F)n1cc(NC(=O)c2cc(-c3cccs3)on2)cn1] |
| 1-19 | 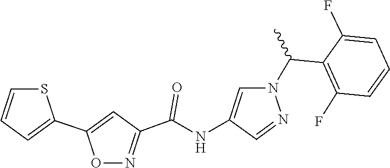 [SMILES: CC(c1c(F)cccc1F)n1cc(NC(=O)c2cc(-c3cccs3)on2)cn1] |
| 1-20 | 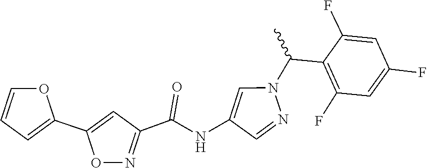 [SMILES: CC(c1c(F)cc(F)cc1F)n1cc(NC(=O)c2cc(-c3ccco3)on2)cn1] |
| 1-21 | 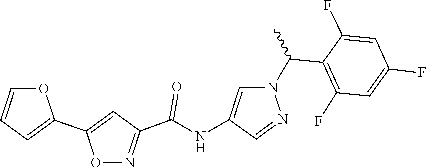 [SMILES: CC(c1c(F)cc(F)cc1F)n1cc(NC(=O)c2cc(-c3ccco3)on2)cn1] |
| 1-22 | 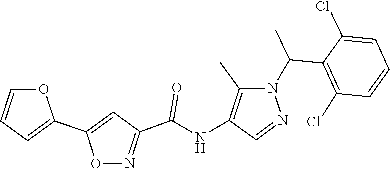 [SMILES: Cc1c(NC(=O)c2cc(-c3ccco3)on2)cnn1C(C)c1c(Cl)cccc1Cl] |
| 1-23 | 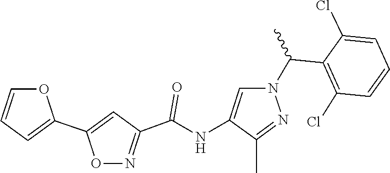 [SMILES: Cc1nn(C(C)c2c(Cl)cccc2Cl)cc1NC(=O)c1cc(-c2ccco2)on1] |
| 1-24 | 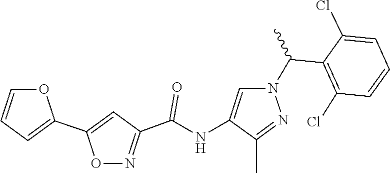 [SMILES: Cc1nn(C(C)c2c(Cl)cccc2Cl)cc1NC(=O)c1cc(-c2ccco2)on1] |
| 1-25 | 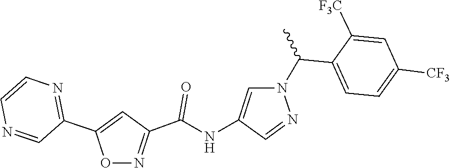 [SMILES: CC(c1ccc(C(F)(F)F)cc1C(F)(F)F)n1cc(NC(=O)c2cc(-c3cnccn3)on2)cn1] |
| 1-26 | 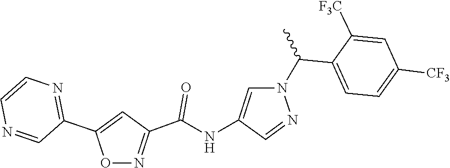 [SMILES: CC(c1ccc(C(F)(F)F)cc1C(F)(F)F)n1cc(NC(=O)c2cc(-c3cnccn3)on2)cn1] |

TABLE 2
Compounds 2-1 to 2-60

| Compound | Structure |
|---|---|
| 2-1 | 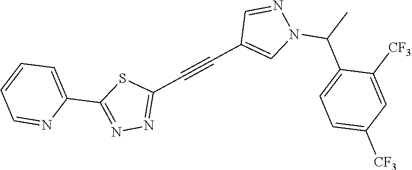 [SMILES: CC(c1ccc(C(F)(F)F)cc1C(F)(F)F)n1cc(C#Cc2nnc(-c3ccccn3)s2)cn1] |
| 2-2 | 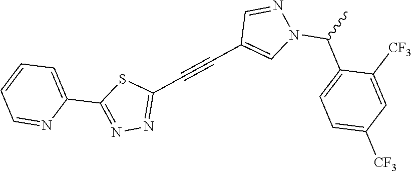 [SMILES: CC(c1ccc(C(F)(F)F)cc1C(F)(F)F)n1cc(C#Cc2nnc(-c3ccccn3)s2)cn1] |
| 2-3 | 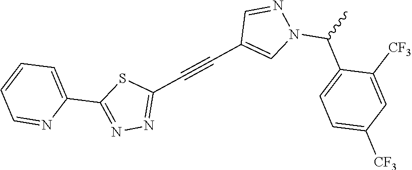 [SMILES: CC(c1ccc(C(F)(F)F)cc1C(F)(F)F)n1cc(C#Cc2nnc(-c3ccccn3)s2)cn1] |
| 2-4 | 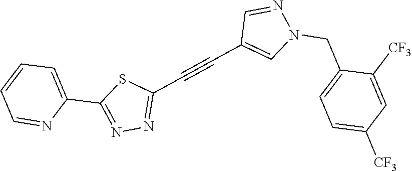 [SMILES: FC(F)(F)c1ccc(Cn2cc(C#Cc3nnc(-c4ccccn4)s3)cn2)c(C(F)(F)F)c1] |
| 2-5 | 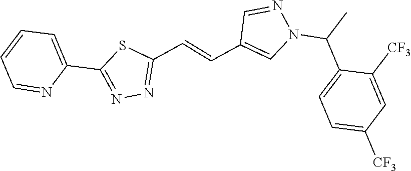 [SMILES: CC(c1ccc(C(F)(F)F)cc1C(F)(F)F)n1cc(/C=C/c2nnc(-c3ccccn3)s2)cn1] |
| 2-6 | 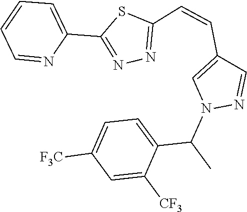 [SMILES: CC(c1ccc(C(F)(F)F)cc1C(F)(F)F)n1cc(/C=C\c2nnc(-c3ccccn3)s2)cn1] |
| 2-7 | 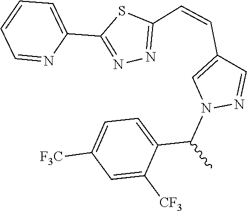 [SMILES: CC(c1ccc(C(F)(F)F)cc1C(F)(F)F)n1cc(/C=C\c2nnc(-c3ccccn3)s2)cn1] |
| 2-8 | 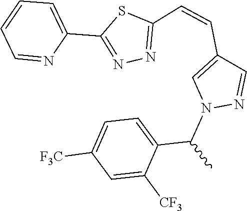 [SMILES: CC(c1ccc(C(F)(F)F)cc1C(F)(F)F)n1cc(/C=C\c2nnc(-c3ccccn3)s2)cn1] |
| 2-9 | 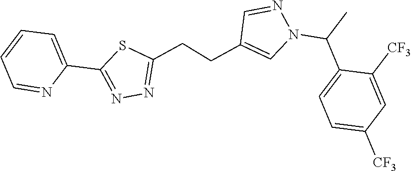 [SMILES: CC(c1ccc(C(F)(F)F)cc1C(F)(F)F)n1cc(CCc2nnc(-c3ccccn3)s2)cn1] |
| 2-10 | 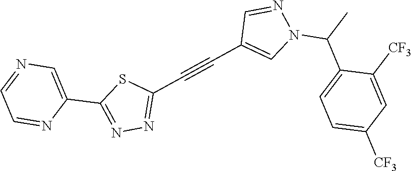 [SMILES: CC(c1ccc(C(F)(F)F)cc1C(F)(F)F)n1cc(C#Cc2nnc(-c3cnccn3)s2)cn1] |
| 2-11 | 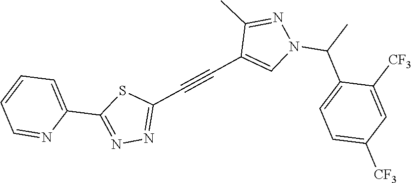 [SMILES: Cc1nn(C(C)c2ccc(C(F)(F)F)cc2C(F)(F)F)cc1C#Cc1nnc(-c2ccccn2)s1] |
| 2-12 | 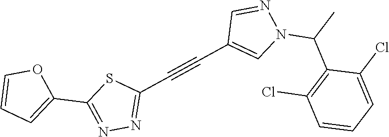 [SMILES: CC(c1c(Cl)cccc1Cl)n1cc(C#Cc2nnc(-c3ccco3)s2)cn1] |
| 2-13 | 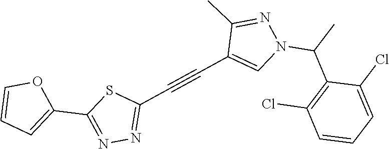 [SMILES: Cc1nn(C(C)c2c(Cl)cccc2Cl)cc1C#Cc1nnc(-c2ccco2)s1] |
| 2-14 | 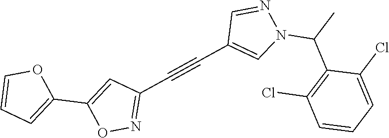 [SMILES: CC(c1c(Cl)cccc1Cl)n1cc(C#Cc2cc(-c3ccco3)on2)cn1] |
| 2-15 | 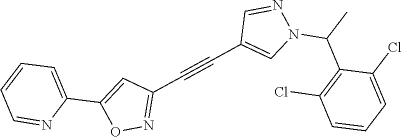 [SMILES: CC(c1c(Cl)cccc1Cl)n1cc(C#Cc2cc(-c3ccccn3)on2)cn1] |
| 2-16 | 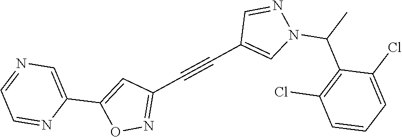 [SMILES: CC(c1c(Cl)cccc1Cl)n1cc(C#Cc2cc(-c3cnccn3)on2)cn1] |
| 2-17 | 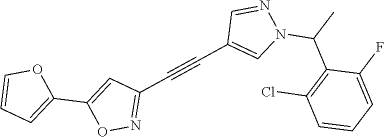 [SMILES: CC(c1c(F)cccc1Cl)n1cc(C#Cc2cc(-c3ccco3)on2)cn1] |
| 2-18 | 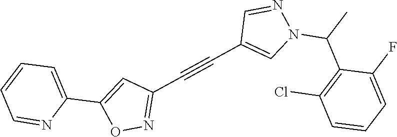 [SMILES: CC(c1c(F)cccc1Cl)n1cc(C#Cc2cc(-c3ccccn3)on2)cn1] |
| 2-19 | 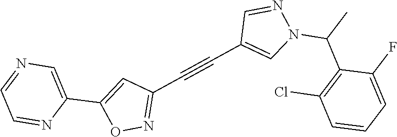 [SMILES: CC(c1c(F)cccc1Cl)n1cc(C#Cc2cc(-c3cnccn3)on2)cn1] |
| 2-20 | 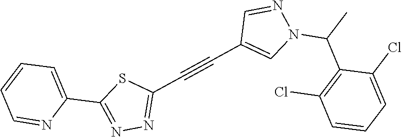 [SMILES: CC(c1c(Cl)cccc1Cl)n1cc(C#Cc2nnc(-c3ccccn3)s2)cn1] |
| 2-21 | 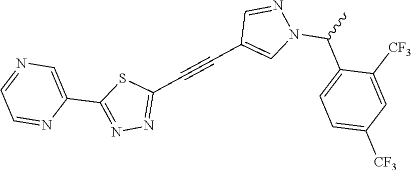 [SMILES: CC(c1ccc(C(F)(F)F)cc1C(F)(F)F)n1cc(C#Cc2nnc(-c3cnccn3)s2)cn1] |
| 2-22 | 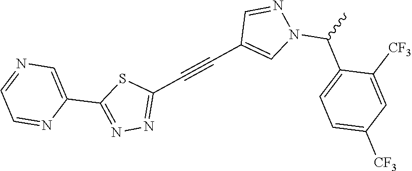 [SMILES: CC(c1ccc(C(F)(F)F)cc1C(F)(F)F)n1cc(C#Cc2nnc(-c3cnccn3)s2)cn1] |
| 2-23 | 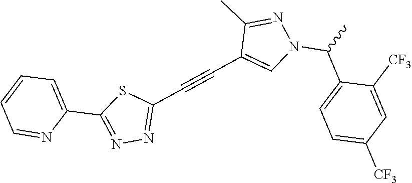 [SMILES: Cc1nn(C(C)c2ccc(C(F)(F)F)cc2C(F)(F)F)cc1C#Cc1nnc(-c2ccccn2)s1] |
| 2-24 | 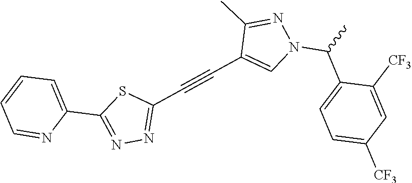 [SMILES: Cc1nn(C(C)c2ccc(C(F)(F)F)cc2C(F)(F)F)cc1C#Cc1nnc(-c2ccccn2)s1] |
| 2-25 | 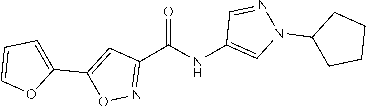 [SMILES: O=C(Nc1cnn(C2CCCC2)c1)c1cc(-c2ccco2)on1] |
| 2-26 | 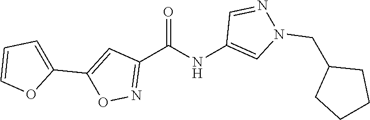 [SMILES: O=C(Nc1cnn(CC2CCCC2)c1)c1cc(-c2ccco2)on1] |
| 2-27 | 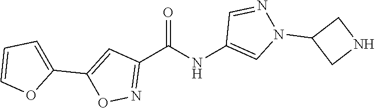 [SMILES: O=C(Nc1cnn(C2CNC2)c1)c1cc(-c2ccco2)on1] |
| 2-28 | 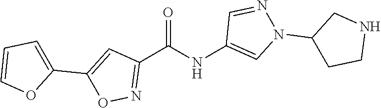 [SMILES: O=C(Nc1cnn(C2CCNC2)c1)c1cc(-c2ccco2)on1] |
| 2-29 | 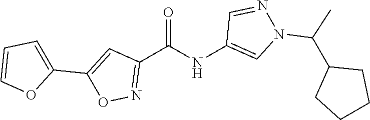 [SMILES: CC(C1CCCC1)n1cc(NC(=O)c2cc(-c3ccco3)on2)cn1] |
| 2-30 | 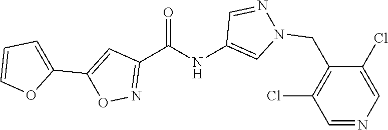 [SMILES: O=C(Nc1cnn(Cc2c(Cl)cncc2Cl)c1)c1cc(-c2ccco2)on1] |
| 2-31 | 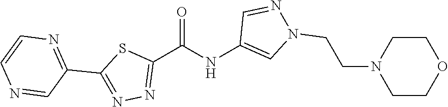 [SMILES: O=C(Nc1cnn(CCN2CCOCC2)c1)c1nnc(-c2cnccn2)s1] |
| 2-32 | 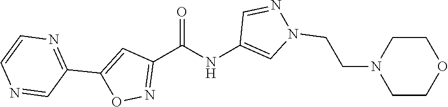 [SMILES: O=C(Nc1cnn(CCN2CCOCC2)c1)c1cc(-c2cnccn2)on1] |
| 2-33 | 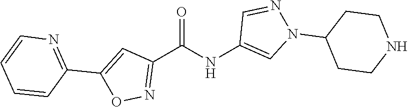 [SMILES: O=C(Nc1cnn(C2CCNCC2)c1)c1cc(-c2ccccn2)on1] |
| 2-34 | 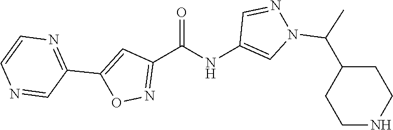 [SMILES: CC(C1CCNCC1)n1cc(NC(=O)c2cc(-c3cnccn3)on2)cn1] |
| 2-35 | 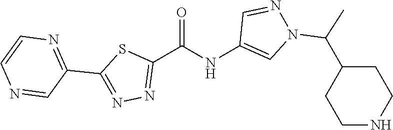 [SMILES: CC(C1CCNCC1)n1cc(NC(=O)c2nnc(-c3cnccn3)s2)cn1] |
| 2-36 | 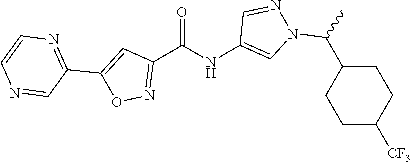 [SMILES: CC(C1CCC(C(F)(F)F)CC1)n1cc(NC(=O)c2cc(-c3cnccn3)on2)cn1] |
| 2-37 | 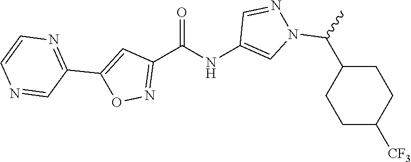 [SMILES: CC(C1CCC(C(F)(F)F)CC1)n1cc(NC(=O)c2cc(-c3cnccn3)on2)cn1] |
| 2-38 | 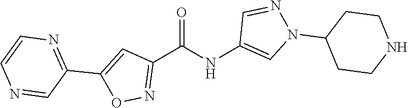 [SMILES: O=C(Nc1cnn(C2CCNCC2)c1)c1cc(-c2cnccn2)on1] |
| 2-39 | 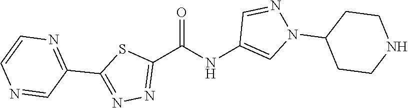 [SMILES: O=C(Nc1cnn(C2CCNCC2)c1)c1nnc(-c2cnccn2)s1] |
| 2-40 | 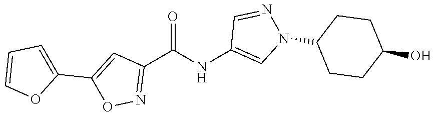 |
| 2-41 | 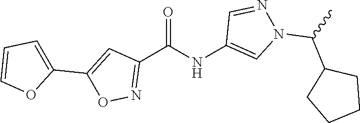 [SMILES: CC(C1CCCC1)n1cc(NC(=O)c2cc(-c3ccco3)on2)cn1] |
| 2-42 | 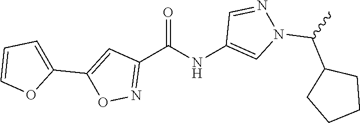 [SMILES: CC(C1CCCC1)n1cc(NC(=O)c2cc(-c3ccco3)on2)cn1] |
| 2-43 | 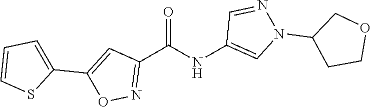 [SMILES: O=C(Nc1cnn(C2CCOC2)c1)c1cc(-c2cccs2)on1] |
| 2-44 | 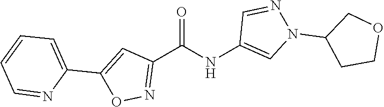 [SMILES: O=C(Nc1cnn(C2CCOC2)c1)c1cc(-c2ccccn2)on1] |
| 2-45 | 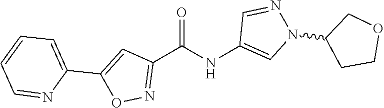 [SMILES: O=C(Nc1cnn(C2CCOC2)c1)c1cc(-c2ccccn2)on1] |
| 2-46 | 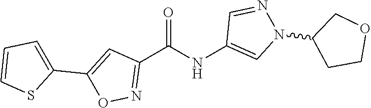 [SMILES: O=C(Nc1cnn(C2CCOC2)c1)c1cc(-c2cccs2)on1] |
| 2-47 | 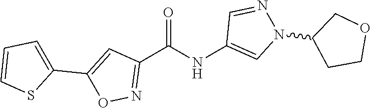 [SMILES: O=C(Nc1cnn(C2CCOC2)c1)c1cc(-c2cccs2)on1] |
| 2-48 | 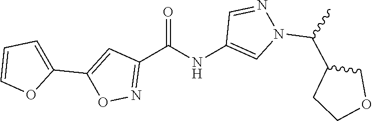 [SMILES: CC(C1CCOC1)n1cc(NC(=O)c2cc(-c3ccco3)on2)cn1] |
| 2-49 | 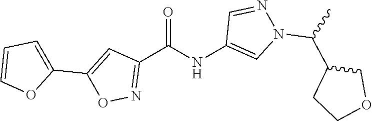 [SMILES: CC(C1CCOC1)n1cc(NC(=O)c2cc(-c3ccco3)on2)cn1] |
| 2-50 | 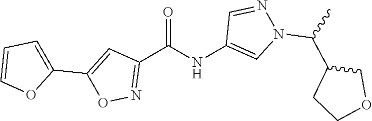 [SMILES: CC(C1CCOC1)n1cc(NC(=O)c2cc(-c3ccco3)on2)cn1] |
| 2-51 | 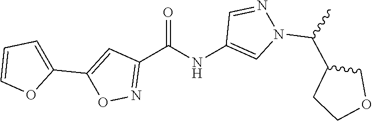 [SMILES: CC(C1CCOC1)n1cc(NC(=O)c2cc(-c3ccco3)on2)cn1] |
| 2-52 | 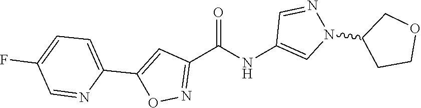 [SMILES: O=C(Nc1cnn(C2CCOC2)c1)c1cc(-c2ccc(F)cn2)on1] |
| 2-53 | 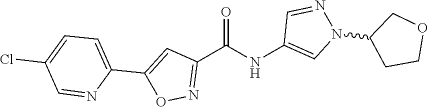 [SMILES: O=C(Nc1cnn(C2CCOC2)c1)c1cc(-c2ccc(Cl)cn2)on1] |
| 2-54 | 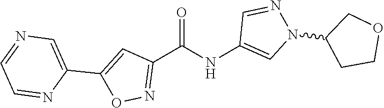 [SMILES: O=C(Nc1cnn(C2CCOC2)c1)c1cc(-c2cnccn2)on1] |
| 2-55 | 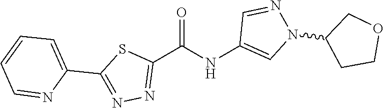 [SMILES: O=C(Nc1cnn(C2CCOC2)c1)c1nnc(-c2ccccn2)s1] |
| 2-562 | 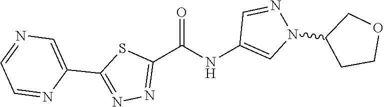 [SMILES: O=C(Nc1cnn(C2CCOC2)c1)c1nnc(-c2cnccn2)s1] |
| 2-57 | 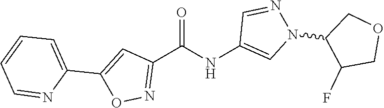 [SMILES: O=C(Nc1cnn(C2COCC2F)c1)c1cc(-c2ccccn2)on1] |
| 2-58 | 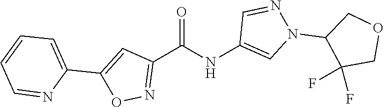 [SMILES: O=C(Nc1cnn(C2COCC2(F)F)c1)c1cc(-c2ccccn2)on1] |
| 2-59 | 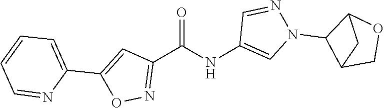 [SMILES: O=C(Nc1cnn(C2C3COC2C3)c1)c1cc(-c2ccccn2)on1] |
| 2-60 | 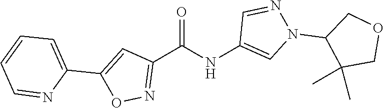 [SMILES: CC1(C)COCC1n1cc(NC(=O)c2cc(-c3ccccn3)on2)cn1] |

TABLE 3
Compounds 3-1 to 3-161

| | |
|---|---|
| 3-1 | 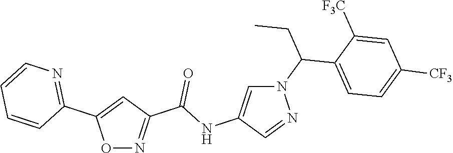 [SMILES: CCC(c1ccc(C(F)(F)F)cc1C(F)(F)F)n1cc(NC(=O)c2cc(-c3ccccn3)on2)cn1] |
| 3-2 | 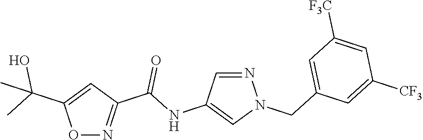 [SMILES: CC(C)(O)c1cc(C(=O)Nc2cnn(Cc3cc(C(F)(F)F)cc(C(F)(F)F)c3)c2)no1] |
| 3-3 | 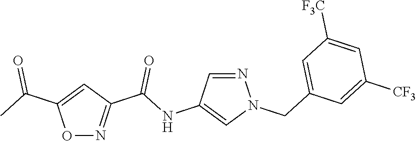 [SMILES: CC(=O)c1cc(C(=O)Nc2cnn(Cc3cc(C(F)(F)F)cc(C(F)(F)F)c3)c2)no1] |
| 3-4 | 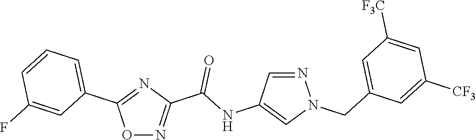 [SMILES: O=C(Nc1cnn(Cc2cc(C(F)(F)F)cc(C(F)(F)F)c2)c1)c1noc(-c2cccc(F)c2)n1] |
| 3-5 | 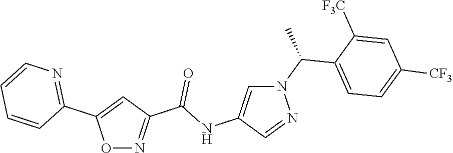 [SMILES: C[C@H](c1ccc(C(F)(F)F)cc1C(F)(F)F)n1cc(NC(=O)c2cc(-c3ccccn3)on2)cn1] |
| 3-6 | 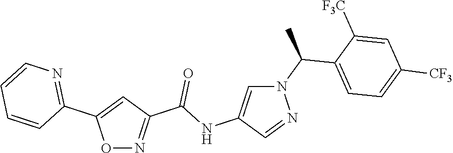 [SMILES: C[C@@H](c1ccc(C(F)(F)F)cc1C(F)(F)F)n1cc(NC(=O)c2cc(-c3ccccn3)on2)cn1] |
| 3-7 | 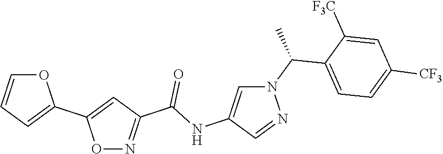 [SMILES: C[C@H](c1ccc(C(F)(F)F)cc1C(F)(F)F)n1cc(NC(=O)c2cc(-c3ccco3)on2)cn1] |
| 3-8 | 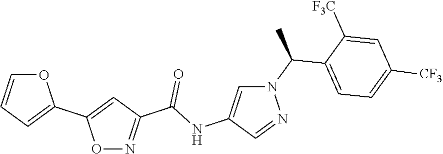 [SMILES: C[C@@H](c1ccc(C(F)(F)F)cc1C(F)(F)F)n1cc(NC(=O)c2cc(-c3ccco3)on2)cn1] |
| 3-9 | 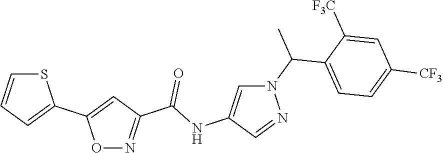 [SMILES: CC(c1ccc(C(F)(F)F)cc1C(F)(F)F)n1cc(NC(=O)c2cc(-c3cccs3)on2)cn1] |
| 3-10 | 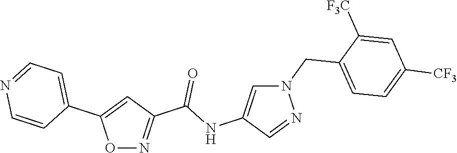 [SMILES: O=C(Nc1cnn(Cc2ccc(C(F)(F)F)cc2C(F)(F)F)c1)c1cc(-c2ccncc2)on1] |
| 3-11 | 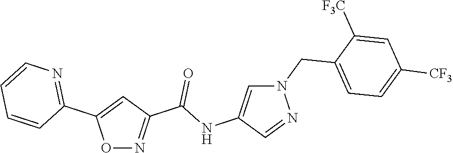 [SMILES: O=C(Nc1cnn(Cc2ccc(C(F)(F)F)cc2C(F)(F)F)c1)c1cc(-c2ccccn2)on1] |
| 3-12 | 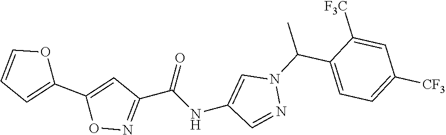 [SMILES: CC(c1ccc(C(F)(F)F)cc1C(F)(F)F)n1cc(NC(=O)c2cc(-c3ccco3)on2)cn1] |
| 3-13 | 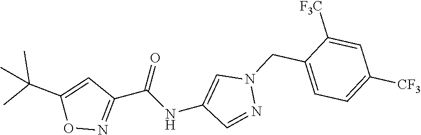 [SMILES: CC(C)(C)c1cc(C(=O)Nc2cnn(Cc3ccc(C(F)(F)F)cc3C(F)(F)F)c2)no1] |
| 3-14 | 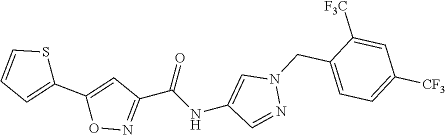 [SMILES: O=C(Nc1cnn(Cc2ccc(C(F)(F)F)cc2C(F)(F)F)c1)c1cc(-c2cccs2)on1] |
| 3-15 | 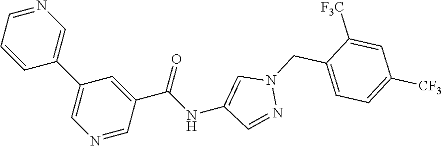 [SMILES: O=C(Nc1cnn(Cc2ccc(C(F)(F)F)cc2C(F)(F)F)c1)c1cncc(-c2cccnc2)c1] |
| 3-16 | 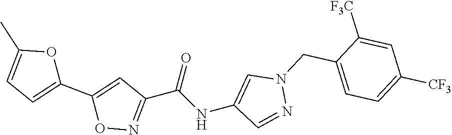 [SMILES: Cc1ccc(-c2cc(C(=O)Nc3cnn(Cc4ccc(C(F)(F)F)cc4C(F)(F)F)c3)no2)o1] |
| 3-17 | 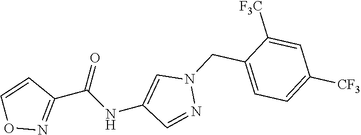 [SMILES: O=C(Nc1cnn(Cc2ccc(C(F)(F)F)cc2C(F)(F)F)c1)c1ccon1] |
| 3-18 | 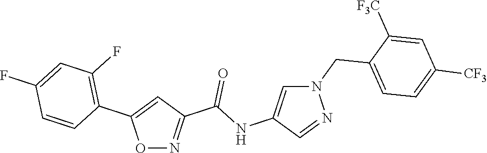 [SMILES: O=C(Nc1cnn(Cc2ccc(C(F)(F)F)cc2C(F)(F)F)c1)c1cc(-c2ccc(F)cc2F)on1] |
| 3-19 (Ceapin A7) | 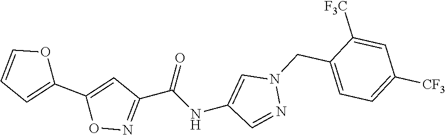 [SMILES: O=C(Nc1cnn(Cc2ccc(C(F)(F)F)cc2C(F)(F)F)c1)c1cc(-c2ccco2)on1] |
| 3-20 | 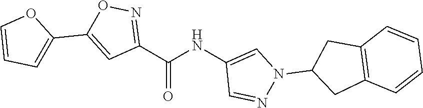 [SMILES: O=C(Nc1cnn(C2Cc3ccccc3C2)c1)c1cc(-c2ccco2)on1] |
| 3-21 | 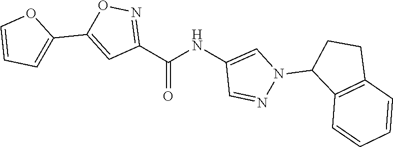 [SMILES: O=C(Nc1cnn(C2CCc3ccccc32)c1)c1cc(-c2ccco2)on1] |
| 3-22 | 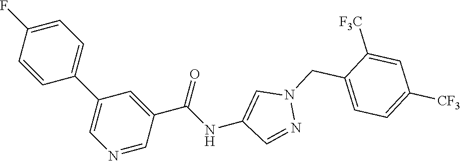 [SMILES: O=C(Nc1cnn(Cc2ccc(C(F)(F)F)cc2C(F)(F)F)c1)c1cncc(-c2ccc(F)cc2)c1] |
| 3-23 | 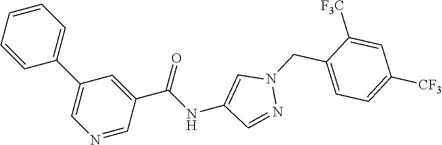 [SMILES: O=C(Nc1cnn(Cc2ccc(C(F)(F)F)cc2C(F)(F)F)c1)c1cncc(-c2ccccc2)c1] |
| 3-24 | 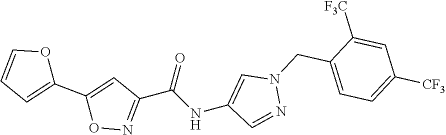 [SMILES: O=C(Nc1cnn(Cc2ccc(C(F)(F)F)cc2C(F)(F)F)c1)c1cc(-c2ccco2)on1] |
| 3-25 | 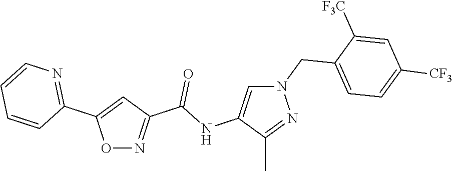 [SMILES: Cc1nn(Cc2ccc(C(F)(F)F)cc2C(F)(F)F)cc1NC(=O)c1cc(-c2ccccn2)on1] |
| 3-26 | 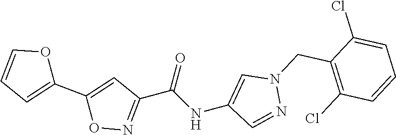 [SMILES: O=C(Nc1cnn(Cc2c(Cl)cccc2Cl)c1)c1cc(-c2ccco2)on1] |
| 3-27 | 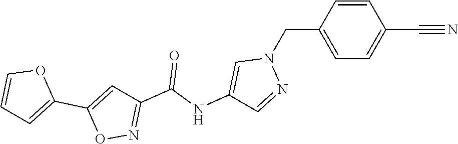 [SMILES: N#Cc1ccc(Cn2cc(NC(=O)c3cc(-c4ccco4)on3)cn2)cc1] |
| 3-28 | 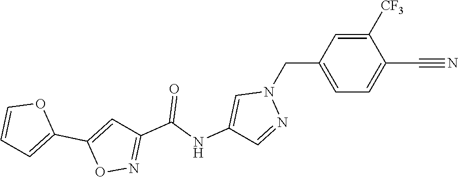 [SMILES: N#Cc1ccc(Cn2cc(NC(=O)c3cc(-c4ccco4)on3)cn2)cc1C(F)(F)F] |
| 3-29 | 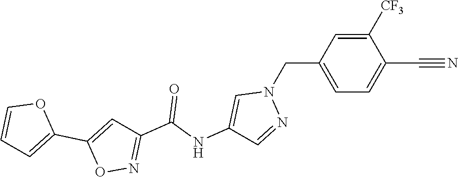 [SMILES: N#Cc1ccc(Cn2cc(NC(=O)c3cc(-c4ccco4)on3)cn2)cc1C(F)(F)F] |
| 3-30 | 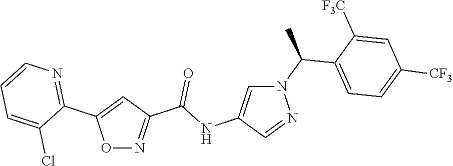 [SMILES: C[C@@H](c1ccc(C(F)(F)F)cc1C(F)(F)F)n1cc(NC(=O)c2cc(-c3ncccc3Cl)on2)cn1] |
| 3-31 | 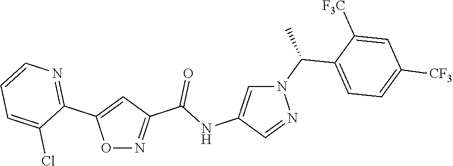 [SMILES: C[C@H](c1ccc(C(F)(F)F)cc1C(F)(F)F)n1cc(NC(=O)c2cc(-c3ncccc3Cl)on2)cn1] |
| 3-32 | 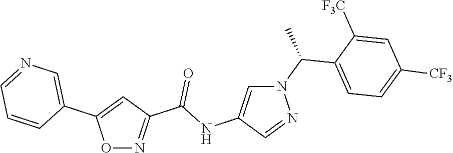 [SMILES: C[C@H](c1ccc(C(F)(F)F)cc1C(F)(F)F)n1cc(NC(=O)c2cc(-c3cccnc3)on2)cn1] |
| 3-33 | 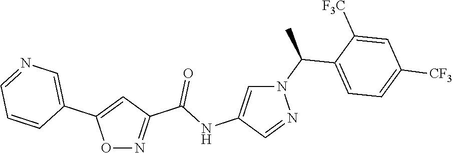 [SMILES: C[C@@H](c1ccc(C(F)(F)F)cc1C(F)(F)F)n1cc(NC(=O)c2cc(-c3cccnc3)on2)cn1] |
| 3-34 | 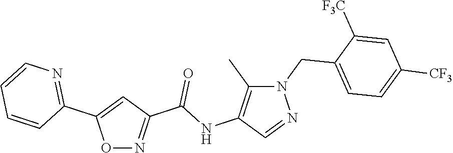 [SMILES: Cc1c(NC(=O)c2cc(-c3ccccn3)on2)cnn1Cc1ccc(C(F)(F)F)cc1C(F)(F)F] |
| 3-35 | 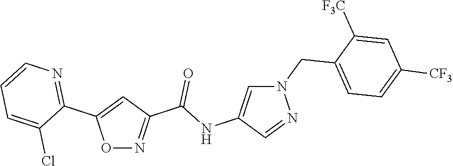 [SMILES: O=C(Nc1cnn(Cc2ccc(C(F)(F)F)cc2C(F)(F)F)c1)c1cc(-c2ncccc2Cl)on1] |
| 3-36 | 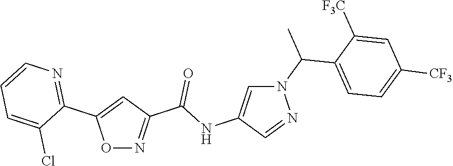 [SMILES: CC(c1ccc(C(F)(F)F)cc1C(F)(F)F)n1cc(NC(=O)c2cc(-c3ncccc3Cl)on2)cn1] |
| 3-37 | 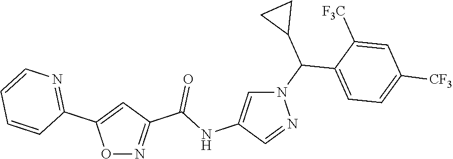 [SMILES: O=C(Nc1cnn(C(c2ccc(C(F)(F)F)cc2C(F)(F)F)C2CC2)c1)c1cc(-c2ccccn2)on1] |
| 3-38 | 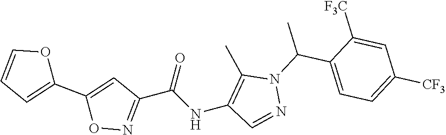 [SMILES: Cc1c(NC(=O)c2cc(-c3ccco3)on2)cnn1C(C)c1ccc(C(F)(F)F)cc1C(F)(F)F] |
| 3-39 | 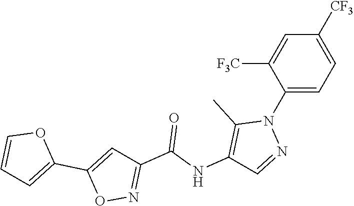 [SMILES: Cc1c(NC(=O)c2cc(-c3ccco3)on2)cnn1-c1ccc(C(F)(F)F)cc1C(F)(F)F] |
| 3-40 | 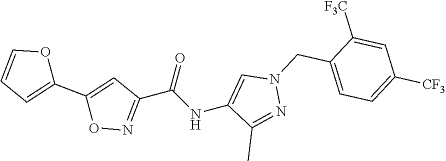 [SMILES: Cc1nn(Cc2ccc(C(F)(F)F)cc2C(F)(F)F)cc1NC(=O)c1cc(-c2ccco2)on1] |
| 3-41 | 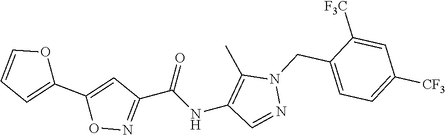 [SMILES: Cc1c(NC(=O)c2cc(-c3ccco3)on2)cnn1Cc1ccc(C(F)(F)F)cc1C(F)(F)F] |
| 3-42 | 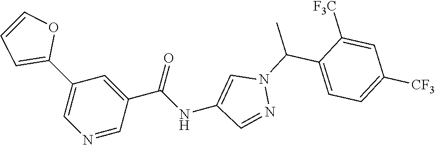 [SMILES: CC(c1ccc(C(F)(F)F)cc1C(F)(F)F)n1cc(NC(=O)c2cncc(-c3ccco3)c2)cn1] |
| 3-43 | 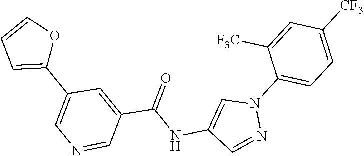 [SMILES: O=C(Nc1cnn(-c2ccc(C(F)(F)F)cc2C(F)(F)F)c1)c1cncc(-c2ccco2)c1] |
| 3-44 | 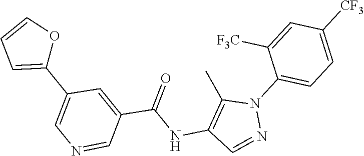 [SMILES: Cc1c(NC(=O)c2cncc(-c3ccco3)c2)cnn1-c1ccc(C(F)(F)F)cc1C(F)(F)F] |
| 3-45 | 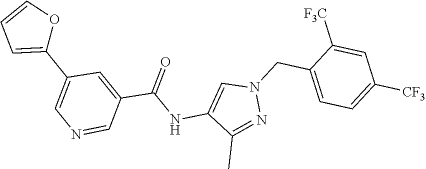 [SMILES: Cc1nn(Cc2ccc(C(F)(F)F)cc2C(F)(F)F)cc1NC(=O)c1cncc(-c2ccco2)c1] |
| 3-46 | 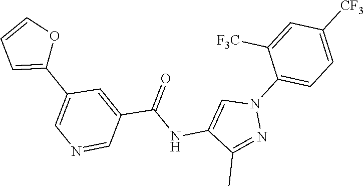 [SMILES: Cc1nn(-c2ccc(C(F)(F)F)cc2C(F)(F)F)cc1NC(=O)c1cncc(-c2ccco2)c1] |
| 3-47 | 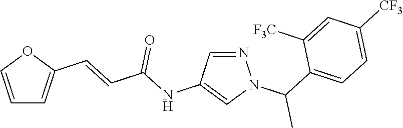 [SMILES: CC(c1ccc(C(F)(F)F)cc1C(F)(F)F)n1cc(NC(=O)/C=C/c2ccco2)cn1] |
| 3-48 | 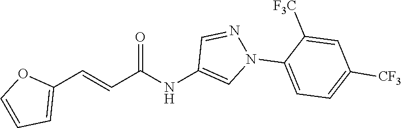 [SMILES: O=C(/C=C/c1ccco1)Nc1cnn(-c2ccc(C(F)(F)F)cc2C(F)(F)F)c1] |
| 3-49 | 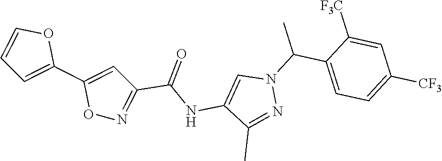 [SMILES: Cc1nn(C(C)c2ccc(C(F)(F)F)cc2C(F)(F)F)cc1NC(=O)c1cc(-c2ccco2)on1] |
| 3-50 | 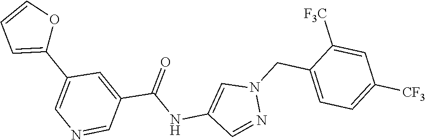 [SMILES: O=C(Nc1cnn(Cc2ccc(C(F)(F)F)cc2C(F)(F)F)c1)c1cncc(-c2ccco2)c1] |
| 3-51 | 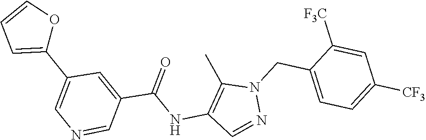 [SMILES: Cc1c(NC(=O)c2cncc(-c3ccco3)c2)cnn1Cc1ccc(C(F)(F)F)cc1C(F)(F)F] |
| 3-52 | 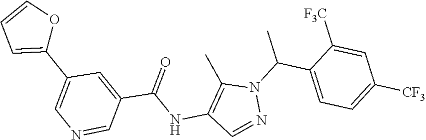 [SMILES: Cc1c(NC(=O)c2cncc(-c3ccco3)c2)cnn1C(C)c1ccc(C(F)(F)F)cc1C(F)(F)F] |
| 3-53 | 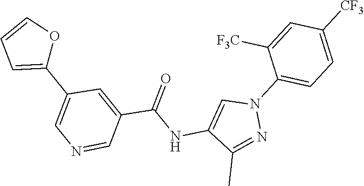 [SMILES: Cc1nn(-c2ccc(C(F)(F)F)cc2C(F)(F)F)cc1NC(=O)c1cncc(-c2ccco2)c1] |
| 3-54 | 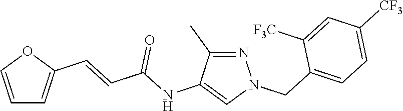 [SMILES: Cc1nn(Cc2ccc(C(F)(F)F)cc2C(F)(F)F)cc1NC(=O)/C=C/c1ccco1] |
| 3-55 | 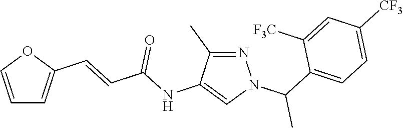 [SMILES: Cc1nn(C(C)c2ccc(C(F)(F)F)cc2C(F)(F)F)cc1NC(=O)/C=C/c1ccco1] |
| 3-56 | 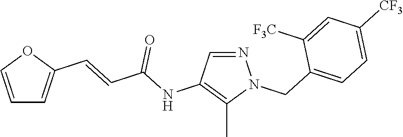 [SMILES: Cc1c(NC(=O)/C=C/c2ccco2)cnn1Cc1ccc(C(F)(F)F)cc1C(F)(F)F] |
| 3-57 | 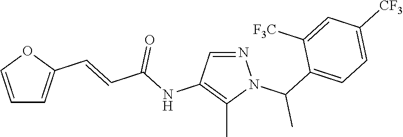 [SMILES: Cc1c(NC(=O)/C=C/c2ccco2)cnn1C(C)c1ccc(C(F)(F)F)cc1C(F)(F)F] |
| 3-58 | 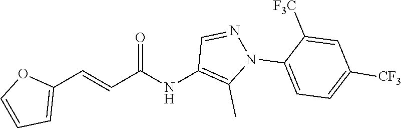 [SMILES: Cc1c(NC(=O)/C=C/c2ccco2)cnn1-c1ccc(C(F)(F)F)cc1C(F)(F)F] |
| 3-59 | 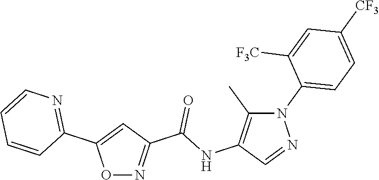 [SMILES: Cc1c(NC(=O)c2cc(-c3ccccn3)on2)cnn1-c1ccc(C(F)(F)F)cc1C(F)(F)F] |
| 3-60 | 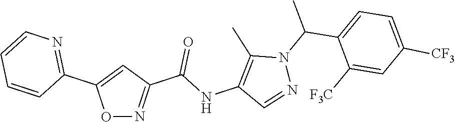 [SMILES: Cc1c(NC(=O)c2cc(-c3ccccn3)on2)cnn1C(C)c1ccc(C(F)(F)F)cc1C(F)(F)F] |
| 3-61 | 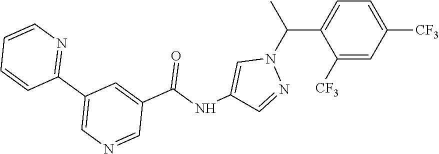 [SMILES: CC(c1ccc(C(F)(F)F)cc1C(F)(F)F)n1cc(NC(=O)c2cncc(-c3ccccn3)c2)cn1] |
| 3-62 | 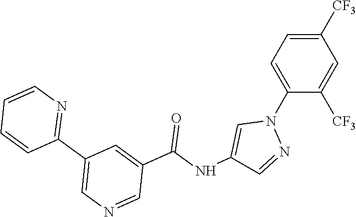 [SMILES: O=C(Nc1cnn(-c2ccc(C(F)(F)F)cc2C(F)(F)F)c1)c1cncc(-c2ccccn2)c1] |
| 3-63 | 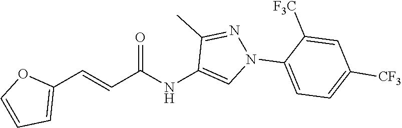 [SMILES: Cc1nn(-c2ccc(C(F)(F)F)cc2C(F)(F)F)cc1NC(=O)/C=C/c1ccco1] |
| 3-64 | 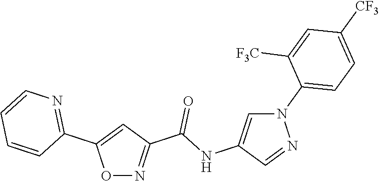 [SMILES: O=C(Nc1cnn(-c2ccc(C(F)(F)F)cc2C(F)(F)F)c1)c1cc(-c2ccccn2)on1] |
| 3-65 | 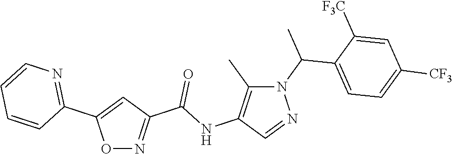 [SMILES: Cc1c(NC(=O)c2cc(-c3ccccn3)on2)cnn1C(C)c1ccc(C(F)(F)F)cc1C(F)(F)F] |
| 3-66 | 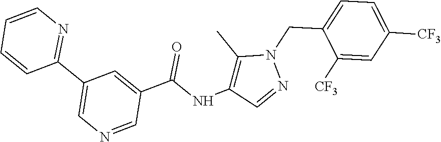 [SMILES: Cc1c(NC(=O)c2cncc(-c3ccccn3)c2)cnn1Cc1ccc(C(F)(F)F)cc1C(F)(F)F] |
| 3-67 | 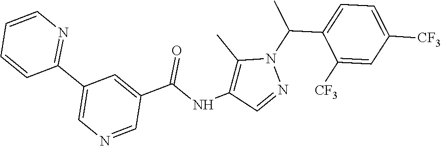 [SMILES: Cc1c(NC(=O)c2cncc(-c3ccccn3)c2)cnn1C(C)c1ccc(C(F)(F)F)cc1C(F)(F)F] |
| 3-68 | 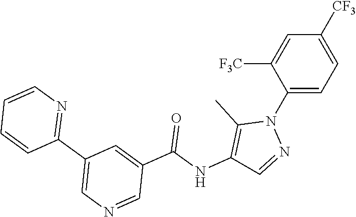 [SMILES: Cc1c(NC(=O)c2cncc(-c3ccccn3)c2)cnn1-c1ccc(C(F)(F)F)cc1C(F)(F)F] |
| 3-69 | 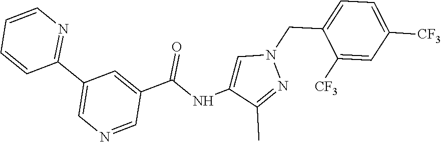 [SMILES: Cc1nn(Cc2ccc(C(F)(F)F)cc2C(F)(F)F)cc1NC(=O)c1cncc(-c2ccccn2)c1] |
| 3-70 | 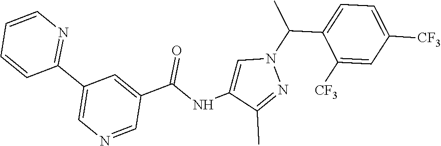 [SMILES: Cc1nn(C(C)c2ccc(C(F)(F)F)cc2C(F)(F)F)cc1NC(=O)c1cncc(-c2ccccn2)c1] |
| 3-71 | 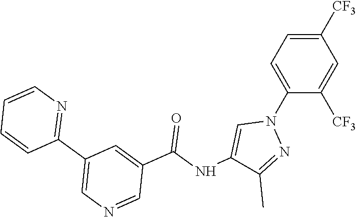 [SMILES: Cc1nn(-c2ccc(C(F)(F)F)cc2C(F)(F)F)cc1NC(=O)c1cncc(-c2ccccn2)c1] |
| 3-72 | 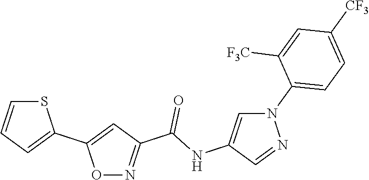 [SMILES: O=C(Nc1cnn(-c2ccc(C(F)(F)F)cc2C(F)(F)F)c1)c1cc(-c2cccs2)on1] |
| 3-73 | 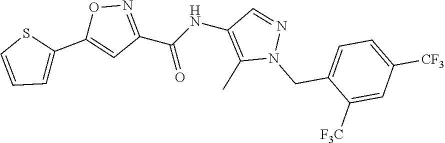 [SMILES: Cc1c(NC(=O)c2cc(-c3cccs3)on2)cnn1Cc1ccc(C(F)(F)F)cc1C(F)(F)F] |
| 3-74 | 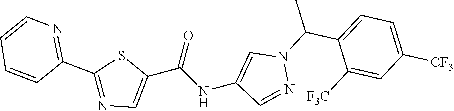 [SMILES: CC(c1ccc(C(F)(F)F)cc1C(F)(F)F)n1cc(NC(=O)c2cnc(-c3ccccn3)s2)cn1] |
| 3-75 | 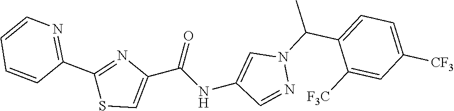 [SMILES: CC(c1ccc(C(F)(F)F)cc1C(F)(F)F)n1cc(NC(=O)c2csc(-c3ccccn3)n2)cn1] |
| 3-76 | 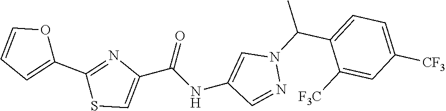 [SMILES: CC(c1ccc(C(F)(F)F)cc1C(F)(F)F)n1cc(NC(=O)c2csc(-c3ccco3)n2)cn1] |
| 3-77 | 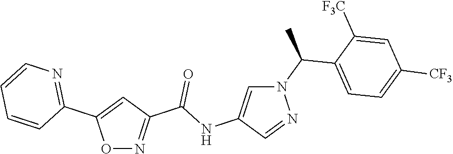 [SMILES: C[C@@H](c1ccc(C(F)(F)F)cc1C(F)(F)F)n1cc(NC(=O)c2cc(-c3ccccn3)on2)cn1] |
| 3-78 | 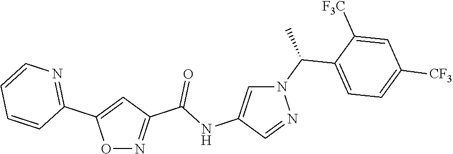 [SMILES: C[C@H](c1ccc(C(F)(F)F)cc1C(F)(F)F)n1cc(NC(=O)c2cc(-c3ccccn3)on2)cn1] |
| 3-79 | 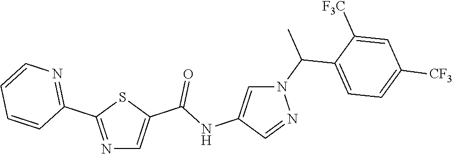 [SMILES: CC(c1ccc(C(F)(F)F)cc1C(F)(F)F)n1cc(NC(=O)c2cnc(-c3ccccn3)s2)cn1] |
| 3-80 | 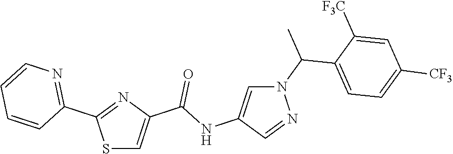 [SMILES: CC(c1ccc(C(F)(F)F)cc1C(F)(F)F)n1cc(NC(=O)c2csc(-c3ccccn3)n2)cn1] |
| 3-81 | 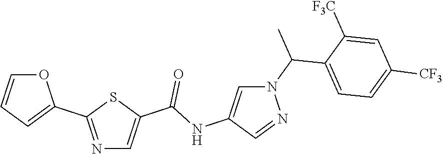 [SMILES: CC(c1ccc(C(F)(F)F)cc1C(F)(F)F)n1cc(NC(=O)c2cnc(-c3ccco3)s2)cn1] |
| 3-82 | 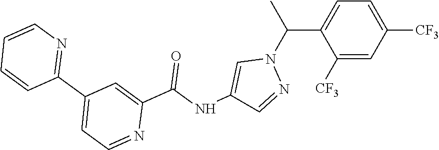 [SMILES: CC(c1ccc(C(F)(F)F)cc1C(F)(F)F)n1cc(NC(=O)c2cc(-c3ccccn3)ccn2)cn1] |
| 3-83 | 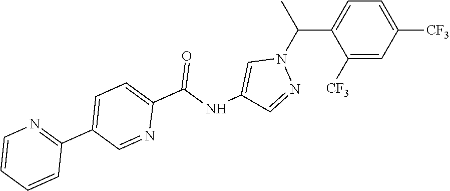 [SMILES: CC(c1ccc(C(F)(F)F)cc1C(F)(F)F)n1cc(NC(=O)c2ccc(-c3ccccn3)cn2)cn1] |
| 3-84 | 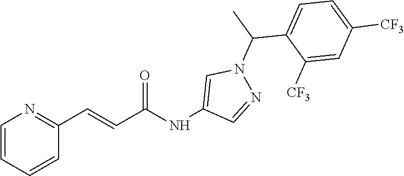 [SMILES: CC(c1ccc(C(F)(F)F)cc1C(F)(F)F)n1cc(NC(=O)/C=C/c2ccccn2)cn1] |
| 3-85 | 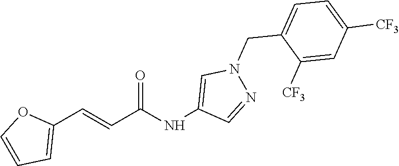 [SMILES: O=C(/C=C/c1ccco1)Nc1cnn(Cc2ccc(C(F)(F)F)cc2C(F)(F)F)c1] |
| 3-86 | 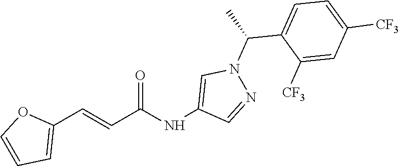 [SMILES: C[C@H](c1ccc(C(F)(F)F)cc1C(F)(F)F)n1cc(NC(=O)/C=C/c2ccco2)cn1] |
| 3-87 | 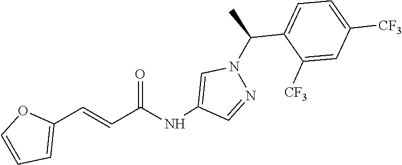 [SMILES: C[C@@H](c1ccc(C(F)(F)F)cc1C(F)(F)F)n1cc(NC(=O)/C=C/c2ccco2)cn1] |
| 3-88 | 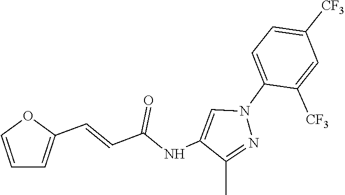 [SMILES: Cc1nn(-c2ccc(C(F)(F)F)cc2C(F)(F)F)cc1NC(=O)/C=C/c1ccco1] |
| 3-89 | 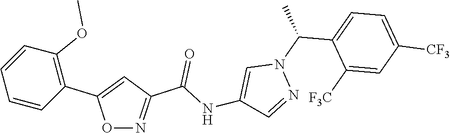 [SMILES: COc1ccccc1-c1cc(C(=O)Nc2cnn([C@H](C)c3ccc(C(F)(F)F)cc3C(F)(F)F)c2)no1] |
| 3-90 | 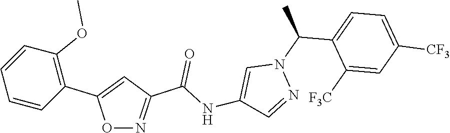 [SMILES: COc1ccccc1-c1cc(C(=O)Nc2cnn([C@@H](C)c3ccc(C(F)(F)F)cc3C(F)(F)F)c2)no1] |
| 3-91 | 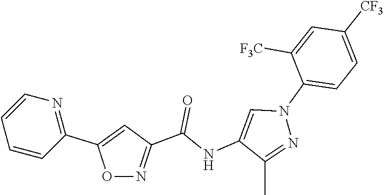 [SMILES: Cc1nn(-c2ccc(C(F)(F)F)cc2C(F)(F)F)cc1NC(=O)c1cc(-c2ccccn2)on1] |
| 3-92 | 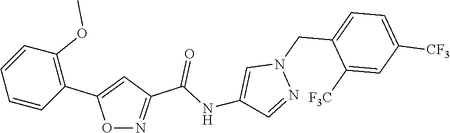 [SMILES: COc1ccccc1-c1cc(C(=O)Nc2cnn(Cc3ccc(C(F)(F)F)cc3C(F)(F)F)c2)no1] |
| 3-93 | 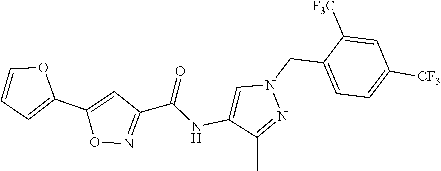 [SMILES: Cc1nn(Cc2ccc(C(F)(F)F)cc2C(F)(F)F)cc1NC(=O)c1cc(-c2ccco2)on1] |
| 3-94 | 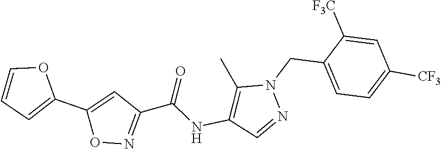 [SMILES: Cc1c(NC(=O)c2cc(-c3ccco3)on2)cnn1Cc1ccc(C(F)(F)F)cc1C(F)(F)F] |
| 3-95 | 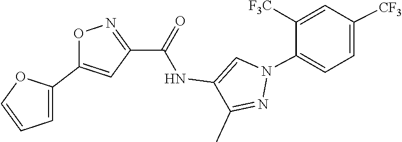 [SMILES: Cc1nn(-c2ccc(C(F)(F)F)cc2C(F)(F)F)cc1NC(=O)c1cc(-c2ccco2)on1] |
| 3-96 | 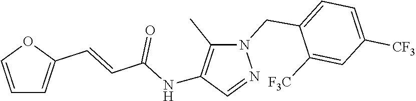 [SMILES: Cc1c(NC(=O)/C=C/c2ccco2)cnn1Cc1ccc(C(F)(F)F)cc1C(F)(F)F] |
| 3-97 | 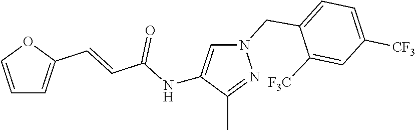 [SMILES: Cc1nn(Cc2ccc(C(F)(F)F)cc2C(F)(F)F)cc1NC(=O)/C=C/c1ccco1] |
| 3-98 | 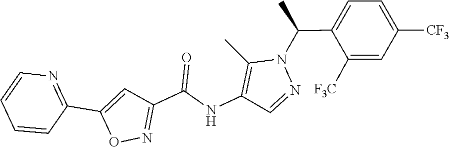 [SMILES: Cc1c(NC(=O)c2cc(-c3ccccn3)on2)cnn1[C@@H](C)c1ccc(C(F)(F)F)cc1C(F)(F)F] |
| 3-99 | 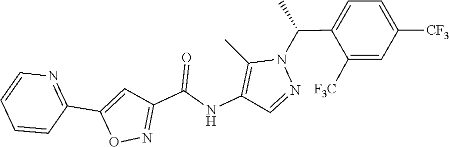 [SMILES: Cc1c(NC(=O)c2cc(-c3ccccn3)on2)cnn1[C@H](C)c1ccc(C(F)(F)F)cc1C(F)(F)F] |
| 3-100 | 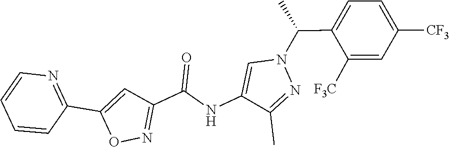 [SMILES: Cc1nn([C@H](C)c2ccc(C(F)(F)F)cc2C(F)(F)F)cc1NC(=O)c1cc(-c2ccccn2)on1] |
| 3-101 | 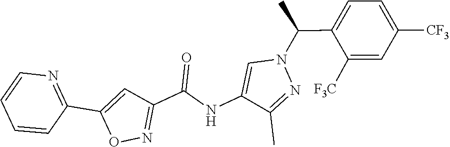 [SMILES: Cc1nn([C@@H](C)c2ccc(C(F)(F)F)cc2C(F)(F)F)cc1NC(=O)c1cc(-c2ccccn2)on1] |
| 3-102 | 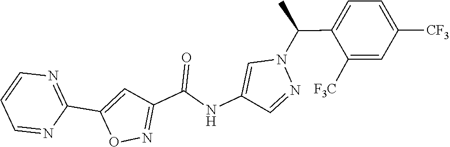 [SMILES: C[C@@H](c1ccc(C(F)(F)F)cc1C(F)(F)F)n1cc(NC(=O)c2cc(-c3ncccn3)on2)cn1] |
| 3-103 | 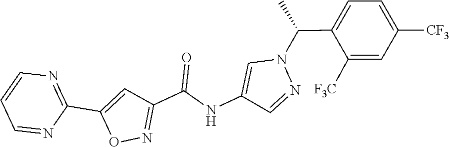 [SMILES: C[C@H](c1ccc(C(F)(F)F)cc1C(F)(F)F)n1cc(NC(=O)c2cc(-c3ncccn3)on2)cn1] |
| 3-104 | 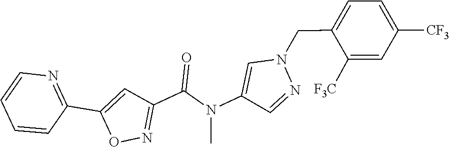 [SMILES: CN(C(=O)c1cc(-c2ccccn2)on1)c1cnn(Cc2ccc(C(F)(F)F)cc2C(F)(F)F)c1] |
| 3-105 | 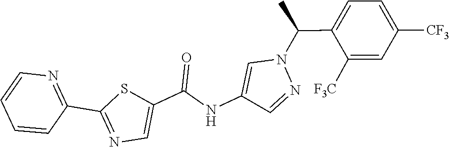 [SMILES: C[C@@H](c1ccc(C(F)(F)F)cc1C(F)(F)F)n1cc(NC(=O)c2cnc(-c3ccccn3)s2)cn1] |
| 3-106 | 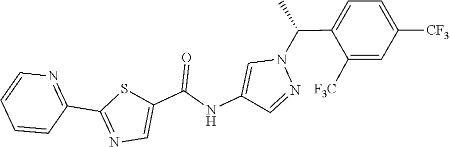 [SMILES: C[C@H](c1ccc(C(F)(F)F)cc1C(F)(F)F)n1cc(NC(=O)c2cnc(-c3ccccn3)s2)cn1] |
| 3-107 | 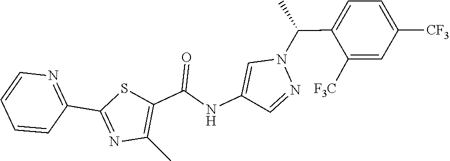 [SMILES: Cc1nc(-c2ccccn2)sc1C(=O)Nc1cnn([C@H](C)c2ccc(C(F)(F)F)cc2C(F)(F)F)c1] |
| 3-108 | 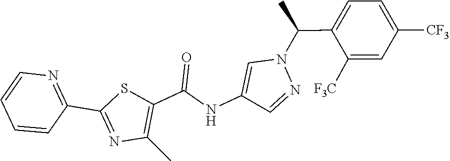 [SMILES: Cc1nc(-c2ccccn2)sc1C(=O)Nc1cnn([C@@H](C)c2ccc(C(F)(F)F)cc2C(F)(F)F)c1] |
| 3-109 | 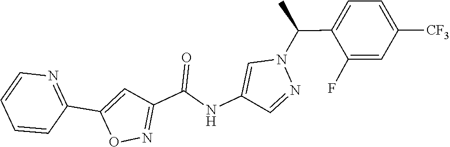 [SMILES: C[C@@H](c1ccc(C(F)(F)F)cc1F)n1cc(NC(=O)c2cc(-c3ccccn3)on2)cn1] |
| 3-110 | 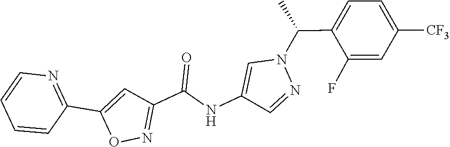 [SMILES: C[C@H](c1ccc(C(F)(F)F)cc1F)n1cc(NC(=O)c2cc(-c3ccccn3)on2)cn1] |
| 3-111 | 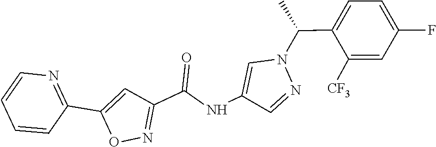 [SMILES: C[C@H](c1ccc(F)cc1C(F)(F)F)n1cc(NC(=O)c2cc(-c3ccccn3)on2)cn1] |
| 3-112 | 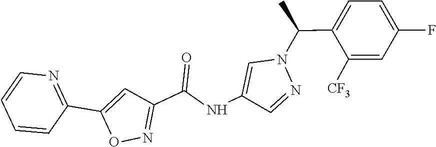 [SMILES: C[C@@H](c1ccc(F)cc1C(F)(F)F)n1cc(NC(=O)c2cc(-c3ccccn3)on2)cn1] |
| 3-113 | 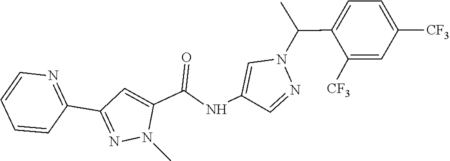 [SMILES: CC(c1ccc(C(F)(F)F)cc1C(F)(F)F)n1cc(NC(=O)c2cc(-c3ccccn3)nn2C)cn1] |
| 3-114 | 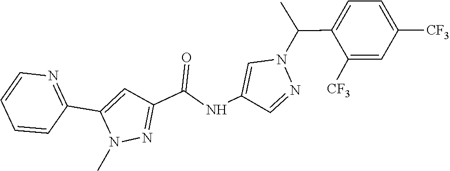 [SMILES: CC(c1ccc(C(F)(F)F)cc1C(F)(F)F)n1cc(NC(=O)c2cc(-c3ccccn3)n(C)n2)cn1] |
| 3-115 | 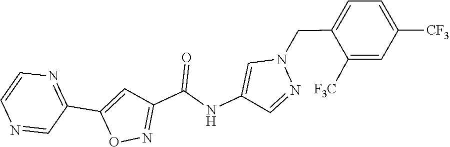 [SMILES: O=C(Nc1cnn(Cc2ccc(C(F)(F)F)cc2C(F)(F)F)c1)c1cc(-c2cnccn2)on1] |
| 3-116 | 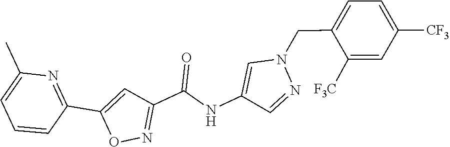 [SMILES: Cc1cccc(-c2cc(C(=O)Nc3cnn(Cc4ccc(C(F)(F)F)cc4C(F)(F)F)c3)no2)n1] |
| 3-117 | 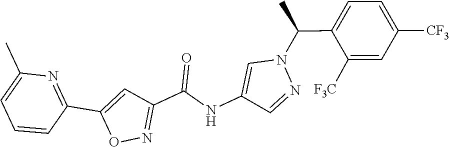 [SMILES: Cc1cccc(-c2cc(C(=O)Nc3cnn([C@@H](C)c4ccc(C(F)(F)F)cc4C(F)(F)F)c3)no2)n1] |
| 3-118 | 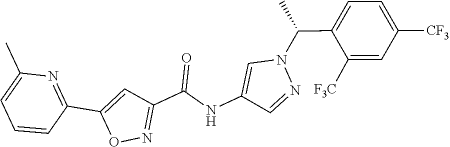 [SMILES: Cc1cccc(-c2cc(C(=O)Nc3cnn([C@H](C)c4ccc(C(F)(F)F)cc4C(F)(F)F)c3)no2)n1] |
| 3-119 | 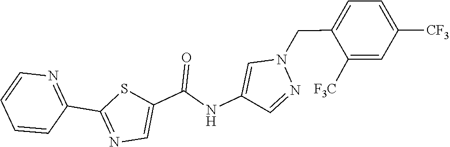 [SMILES: O=C(Nc1cnn(Cc2ccc(C(F)(F)F)cc2C(F)(F)F)c1)c1cnc(-c2ccccn2)s1] |
| 3-120 | 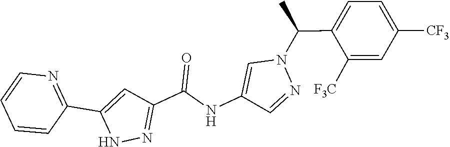 [SMILES: C[C@@H](c1ccc(C(F)(F)F)cc1C(F)(F)F)n1cc(NC(=O)c2cc(-c3ccccn3)[nH]n2)cn1] |
| 3-121 | 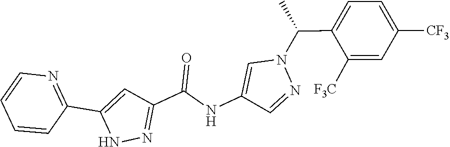 [SMILES: C[C@H](c1ccc(C(F)(F)F)cc1C(F)(F)F)n1cc(NC(=O)c2cc(-c3ccccn3)[nH]n2)cn1] |
| 3-122 | 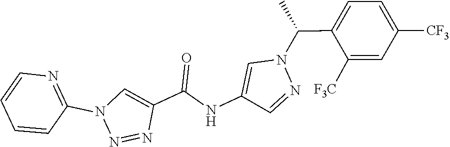 [SMILES: C[C@H](c1ccc(C(F)(F)F)cc1C(F)(F)F)n1cc(NC(=O)c2cn(-c3ccccn3)nn2)cn1] |
| 3-123 | 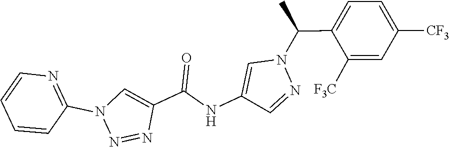 [SMILES: C[C@@H](c1ccc(C(F)(F)F)cc1C(F)(F)F)n1cc(NC(=O)c2cn(-c3ccccn3)nn2)cn1] |
| 3-124 | 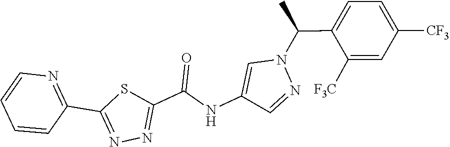 [SMILES: C[C@@H](c1ccc(C(F)(F)F)cc1C(F)(F)F)n1cc(NC(=O)c2nnc(-c3ccccn3)s2)cn1] |
| 3-125 | 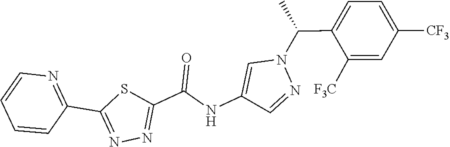 [SMILES: C[C@H](c1ccc(C(F)(F)F)cc1C(F)(F)F)n1cc(NC(=O)c2nnc(-c3ccccn3)s2)cn1] |
| 3-126 | 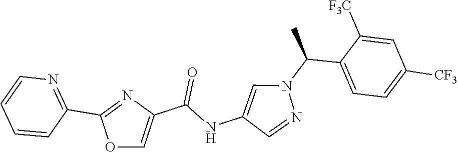 [SMILES: C[C@@H](c1ccc(C(F)(F)F)cc1C(F)(F)F)n1cc(NC(=O)c2coc(-c3ccccn3)n2)cn1] |
| 3-127 | 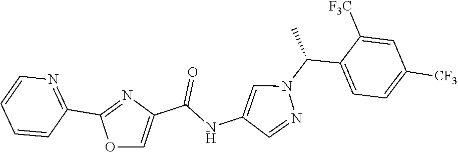 [SMILES: C[C@H](c1ccc(C(F)(F)F)cc1C(F)(F)F)n1cc(NC(=O)c2coc(-c3ccccn3)n2)cn1] |
| 3-128 | 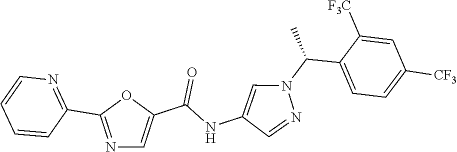 [SMILES: C[C@H](c1ccc(C(F)(F)F)cc1C(F)(F)F)n1cc(NC(=O)c2cnc(-c3ccccn3)o2)cn1] |
| 3-129 | 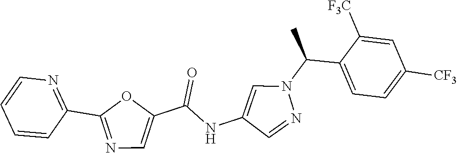 [SMILES: C[C@@H](c1ccc(C(F)(F)F)cc1C(F)(F)F)n1cc(NC(=O)c2cnc(-c3ccccn3)o2)cn1] |
| 3-130 | 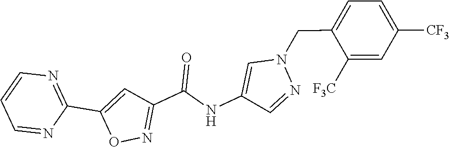 [SMILES: O=C(Nc1cnn(Cc2ccc(C(F)(F)F)cc2C(F)(F)F)c1)c1cc(-c2ncccn2)on1] |
| 3-131 | 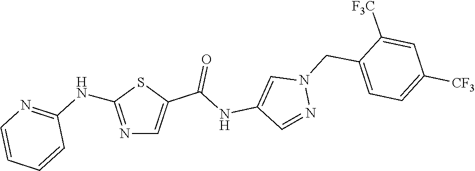 [SMILES: O=C(Nc1cnn(Cc2ccc(C(F)(F)F)cc2C(F)(F)F)c1)c1cnc(Nc2ccccn2)s1] |
| 3-132 | 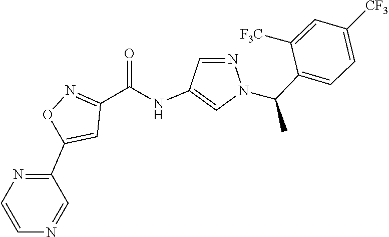 [SMILES: C[C@H](c1ccc(C(F)(F)F)cc1C(F)(F)F)n1cc(NC(=O)c2cc(-c3cnccn3)on2)cn1] |
| 3-133 | 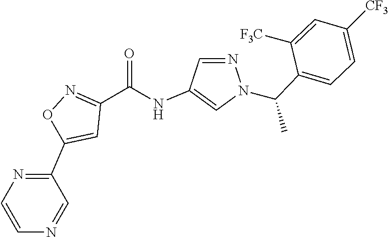 [SMILES: C[C@@H](c1ccc(C(F)(F)F)cc1C(F)(F)F)n1cc(NC(=O)c2cc(-c3cnccn3)on2)cn1] |
| 3-134 | 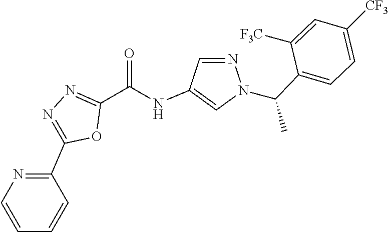 [SMILES: C[C@@H](c1ccc(C(F)(F)F)cc1C(F)(F)F)n1cc(NC(=O)c2nnc(-c3ccccn3)o2)cn1] |
| 3-135 | 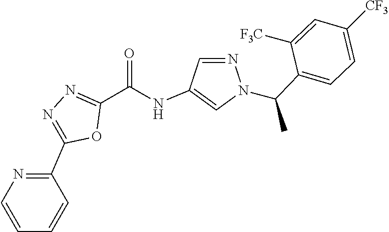 [SMILES: C[C@H](c1ccc(C(F)(F)F)cc1C(F)(F)F)n1cc(NC(=O)c2nnc(-c3ccccn3)o2)cn1] |
| 3-136 | 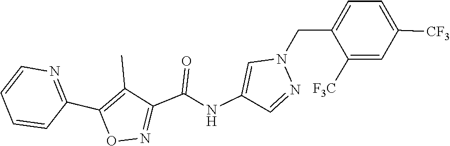 [SMILES: Cc1c(C(=O)Nc2cnn(Cc3ccc(C(F)(F)F)cc3C(F)(F)F)c2)noc1-c1ccccn1] |
| 3-137 | 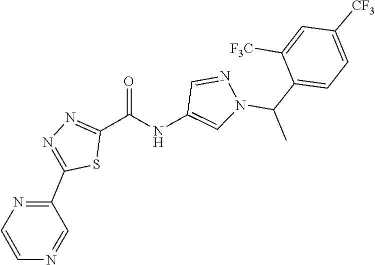 [SMILES: CC(c1ccc(C(F)(F)F)cc1C(F)(F)F)n1cc(NC(=O)c2nnc(-c3cnccn3)s2)cn1] |
| 3-138 | 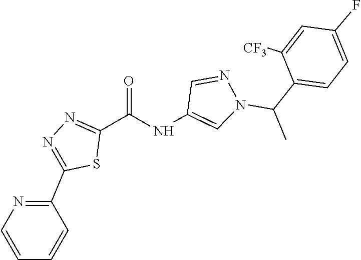 [SMILES: CC(c1ccc(F)cc1C(F)(F)F)n1cc(NC(=O)c2nnc(-c3ccccn3)s2)cn1] |
| 3-139 | 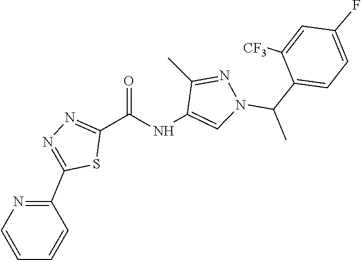 [SMILES: Cc1nn(C(C)c2ccc(F)cc2C(F)(F)F)cc1NC(=O)c1nnc(-c2ccccn2)s1] |
| 3-140 | 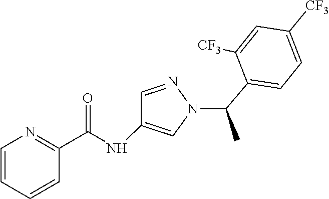 [SMILES: C[C@H](c1ccc(C(F)(F)F)cc1C(F)(F)F)n1cc(NC(=O)c2ccccn2)cn1] |
| 3-141 | 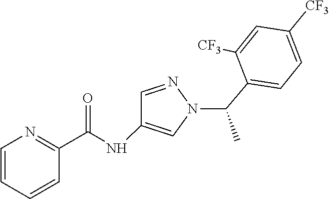 [SMILES: C[C@@H](c1ccc(C(F)(F)F)cc1C(F)(F)F)n1cc(NC(=O)c2ccccn2)cn1] |
| 3-142 | 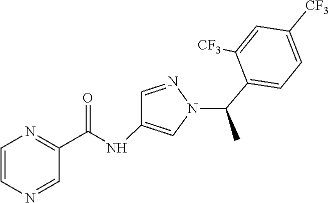 [SMILES: C[C@H](c1ccc(C(F)(F)F)cc1C(F)(F)F)n1cc(NC(=O)c2cnccn2)cn1] |
| 3-143 | 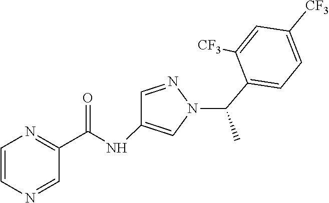 [SMILES: C[C@@H](c1ccc(C(F)(F)F)cc1C(F)(F)F)n1cc(NC(=O)c2cnccn2)cn1] |
| 3-144 | 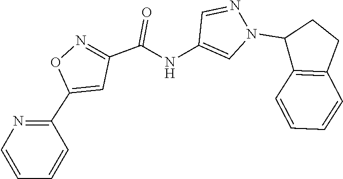 [SMILES: O=C(Nc1cnn(C2CCc3ccccc32)c1)c1cc(-c2ccccn2)on1] |
| 3-145 | 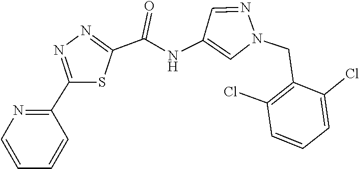 [SMILES: O=C(Nc1cnn(Cc2c(Cl)cccc2Cl)c1)c1nnc(-c2ccccn2)s1] |
| 3-146 | 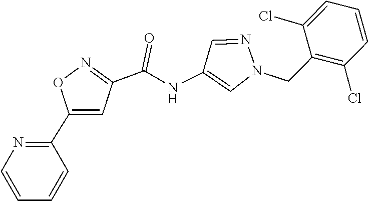 [SMILES: O=C(Nc1cnn(Cc2c(Cl)cccc2Cl)c1)c1cc(-c2ccccn2)on1] |
| 3-147 | 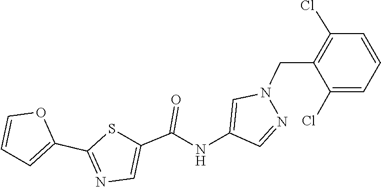 [SMILES: O=C(Nc1cnn(Cc2c(Cl)cccc2Cl)c1)c1cnc(-c2ccco2)s1] |
| 3-148 | 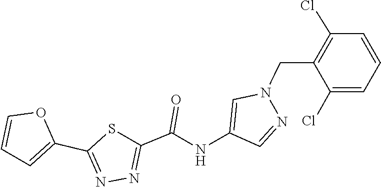 [SMILES: O=C(Nc1cnn(Cc2c(Cl)cccc2Cl)c1)c1nnc(-c2ccco2)s1] |
| 3-149 | 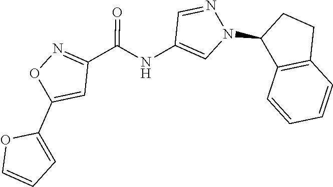 [SMILES: O=C(Nc1cnn([C@H]2CCc3ccccc32)c1)c1cc(-c2ccco2)on1] |
| 3-150 | 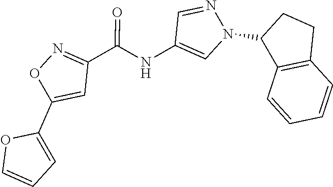 |
| 3-151 | 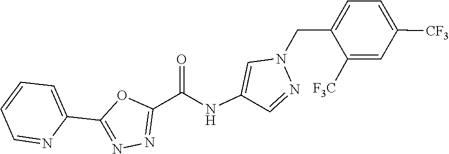 [SMILES: O=C(Nc1cnn(Cc2ccc(C(F)(F)F)cc2C(F)(F)F)c1)c1nnc(-c2ccccn2)o1] |
| 3-152 | 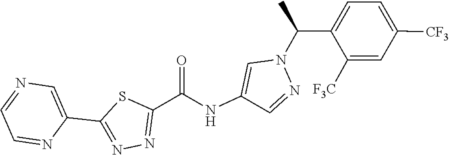 [SMILES: C[C@@H](c1ccc(C(F)(F)F)cc1C(F)(F)F)n1cc(NC(=O)c2nnc(-c3cnccn3)s2)cn1] |
| 3-153 | 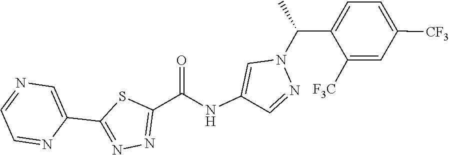 [SMILES: C[C@H](c1ccc(C(F)(F)F)cc1C(F)(F)F)n1cc(NC(=O)c2nnc(-c3cnccn3)s2)cn1] |
| 3-154 | 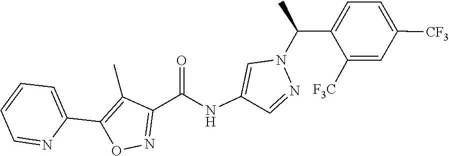 [SMILES: Cc1c(C(=O)Nc2cnn([C@@H](C)c3ccc(C(F)(F)F)cc3C(F)(F)F)c2)noc1-c1ccccn1] |
| 3-155 | 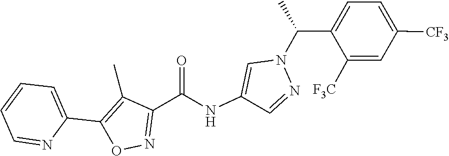 [SMILES: Cc1c(C(=O)Nc2cnn([C@H](C)c3ccc(C(F)(F)F)cc3C(F)(F)F)c2)noc1-c1ccccn1] |
| 3-156 | 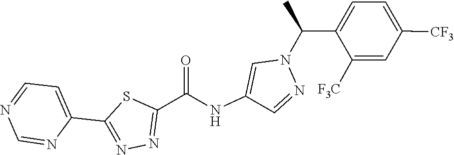 [SMILES: C[C@@H](c1ccc(C(F)(F)F)cc1C(F)(F)F)n1cc(NC(=O)c2nnc(-c3ccncn3)s2)cn1] |
| 3-157 | 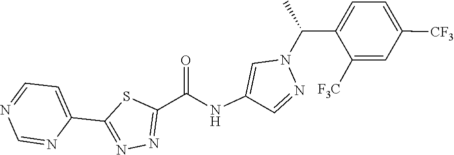 [SMILES: C[C@H](c1ccc(C(F)(F)F)cc1C(F)(F)F)n1cc(NC(=O)c2nnc(-c3ccncn3)s2)cn1] |
| 3-158 | 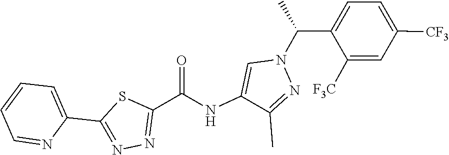 [SMILES: Cc1nn([C@H](C)c2ccc(C(F)(F)F)cc2C(F)(F)F)cc1NC(=O)c1nnc(-c2ccccn2)s1] |
| 3-159 | 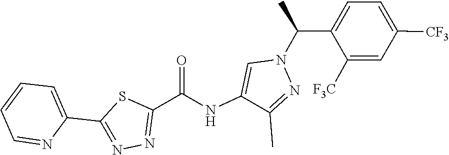 [SMILES: Cc1nn([C@@H](C)c2ccc(C(F)(F)F)cc2C(F)(F)F)cc1NC(=O)c1nnc(-c2ccccn2)s1] |
| 3-160 | 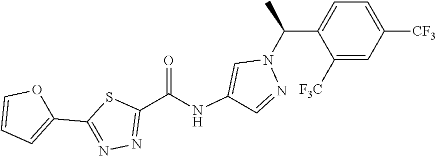 [SMILES: C[C@@H](c1ccc(C(F)(F)F)cc1C(F)(F)F)n1cc(NC(=O)c2nnc(-c3ccco3)s2)cn1] |
| 3-161 | 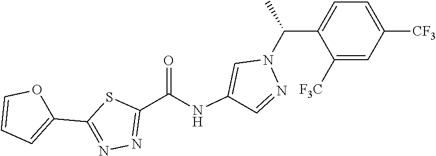 [SMILES: C[C@H](c1ccc(C(F)(F)F)cc1C(F)(F)F)n1cc(NC(=O)c2nnc(-c3ccco3)s2)cn1] |

What is claimed is:

1. A method of treating a subject having a hepatic condition, the method comprising:
administering a pharmaceutical composition comprising a therapeutically effective amount of a synthetic activating transcription factor inhibitor (ATFi), wherein the synthetic ATFi modulates a signaling of an activating transcription factor (ATF) in a cell via interference with a cellular pathway.

2. The method of claim 1, wherein the interference with the cellular pathway comprises sequestering an ATF protein as a transmembrane protein in an endoplasmic reticulum, preventing trafficking of the ATF protein from the endoplasmic reticulum to a Golgi apparatus, cleaving the ATF protein via one or more proteases, or blocking trafficking of a soluble and active ATF fragment to a nucleus of the cell, or a combination thereof.

3. The method of claim 1 or claim 2, wherein the synthetic ATFi is a synthetic antisense oligonucleotide.

4. The method of claim 3, wherein the synthetic antisense oligonucleotide knocks down a nucleic acid transcript, thereby reducing levels of a targeted protein via binding to a target mRNA.

5. The method of claim 3 or claim 4, wherein the synthetic antisense oligonucleotide is conjugated to a triantennary N-acetyl galactosamine (GalNAc).

6. The method of claim 5, wherein the GalNAc targets asialoglycoprotein receptors.

7. The method of any one of claims 1-6, wherein the cell comprises a hepatocyte.

8. The method of any one of claims 1-7, wherein the hepatic condition is non-alcoholic fatty liver disease (NAFLD), non-alcoholic steatohepatitis (NASH), hepatocyte ballooning, hepatocyte cell death and fibrosis, or cirrhosis and hepatocellular carcinoma.

9. The method of any one of claims 1-8, wherein the pharmaceutical composition is administered to the subject via subcutaneous injection, intramuscular injection, or intravenous injection.

10. The method of any one of claims 1-9, wherein the ATF is ATF6.

11. A method of detecting a hepatic condition in a subject, the method comprising:
administering a therapeutically effective amount of a pharmaceutical composition to the subject suspected of having the hepatic condition, wherein the pharmaceutical composition comprises an activating transcription factor inhibitor (ATFi) configured to modulate an activating transcription factor (ATF);
determining an efficacy of the ATFi via comparing a first level of the ATF before administering the ATFi to the subject and a second level of the ATF after administering the ATFi to the subject; and
measuring a biomarker level associated with the hepatic condition after administering the ATFi to the subject,
thereby detecting a presence or absence of the hepatic condition.

12. The method of claim 11, wherein the biomarker is screened via digital spatial profiling.

13. The method of claim 11 or claim 12, wherein the ATFi is an antisense oligonucleotide.

14. The method of claim 11 or claim 12, wherein the ATFi is a small-molecule inhibitor.

15. The method of claim 14, wherein the small molecule inhibitor is Ceapin A7, or a pharmaceutically acceptable salt thereof.

16. The method of claim 14, wherein the small molecule inhibitor comprises a compound of Table 1, Table 2, or Table 3, or a pharmaceutically acceptable salt thereof.

17. The method of any one of claims 11-16, wherein determining the efficacy of the ATFi is performed in vitro.

18. The method of any one of claims 11-16, wherein the determining the efficacy of the ATFi is performed in vivo.

19. The method of any one of claims 11-18, wherein the hepatic condition is non-alcoholic fatty liver disease (NAFLD), non-alcoholic steatohepatitis (NASH), hepatocyte ballooning, hepatocyte cell death and fibrosis, cirrhosis, hepatocellular carcinoma, cholangiocarcinoma, metastatic liver cancer, or any combination thereof.

20. The method of any one of claims 11-19, wherein the pharmaceutical composition is administered to the subject via oral administration, subcutaneous injection, intramuscular injection, or intravenous injection.

21. The method of any one of claims 11-20, wherein the ATF is ATF6.

22. A method of inhibiting expression of an activating transcription factor (ATF) gene in a cell of a subject, the method comprising administering to the subject a therapeutically effective amount of a pharmaceutical composition comprising a synthetic activating transcription factor inhibitor (ATFi) and a suitable delivery vehicle.

23. The method of claim 22, wherein the synthetic ATFi is a synthetic antisense oligonucleotide.

24. The method of claim 23, wherein the synthetic antisense oligonucleotide knocks down a gene encoding the ATF via binding to a target mRNA.

25. The method of any one of claims 22-24, wherein the ATF is ATF6.

26. The method of any one of claims 23-25, wherein the synthetic antisense oligonucleotide is conjugated to a triantennary N-acetyl galactosamine (GalNAc).

27. The method of claim 26, wherein the GalNAc targets asialoglycoprotein receptors.

28. The method of any one of claims 22-27, wherein the cell is a hepatocyte.

29. The method of claim 28, wherein the subject has a hepatic condition.

30. The method of claim 28, wherein the hepatic condition is non-alcoholic fatty liver disease (NAFLD), non-alcoholic steatohepatitis (NASH), hepatocyte ballooning, hepatocyte cell death and fibrosis, cirrhosis, or liver cancer, or any combination thereof.

31. The method of any one of claims 22-30, wherein the pharmaceutical composition is administered to the subject via subcutaneous injection, intramuscular injection, or intravenous injection.

32. The method of any one of claims 22-31, wherein the pharmaceutical composition further comprises a suitable delivery vehicle comprising liposomes, nanoparticles, microparticles, microspheres, lipid particles, vesicles, polyaxamer, or polycationic material.

33. A method of inhibiting an activating transcription factor (ATF) in a cell of a subject, the method comprising:
administering to the subject a therapeutically effective amount of a pharmaceutical composition comprising a synthetic activating transcription factor inhibitor (ATFi) and a suitable delivery vehicle, wherein the synthetic ATFi, following administration to the subject, couples to a receptor in the cell,
thereby inhibiting the ATF in the cell.

34. The method of claim 33, wherein the synthetic ATFi is a synthetic antisense oligonucleotide.

35. The method of claim 34, wherein the synthetic antisense oligonucleotide knocks down an endonuclease transcript and reduces levels of a targeted protein via binding to a target mRNA.

36. The method of claim 35, wherein the targeted protein is an ATF6 protein.

37. The method of any one of claims 34-36, wherein the synthetic antisense oligonucleotide is conjugated to a triantennary N-acetyl galactosamine (GalNAc).

38. The method of claim 37, wherein the GalNAc targets asialoglycoprotein receptors.

39. The method of any one of claims 33-38, wherein the cell is a hepatocyte.

40. The method of any one of claims 33-39, wherein the subject has a hepatic condition.

41. The method of claim 40, wherein the hepatic condition is non-alcoholic fatty liver disease (NAFLD), non-alcoholic steatohepatitis (NASH), hepatocyte ballooning, hepatocyte cell death and fibrosis, cirrhosis, or hepatocellular carcinoma.

42. The method of any one of claims 33-41, wherein the pharmaceutical composition is administered to the subject via subcutaneous injection, intramuscular injection, or intravenous injection.

43. The method of any one of claims 33-42, wherein the suitable delivery vehicle is selected from the group consisting essentially of liposomes, nanoparticles, microparticles, microspheres, lipid particles, vesicles, polyaxamer, native, labeled or synthetic proteins, and polycationic materials.

44. The method of any one of claims 1-43, wherein the subject is a human.

45. The method of any one of claims 1-44, wherein the subject is over 30, 40, 50, 60, 70 or 80 years of age, or an age within a range defined by of any of the preceding numbers.

46. The method of any one of claims 1, 2, 7-12, 17-22, 28-33 and 39-45, wherein the ATFi is a small-molecule inhibitor.

47. The method of claim 46, wherein the small molecule inhibitor is Ceapin A7, or a pharmaceutically acceptable salt thereof.

48. The method of claim 46, wherein the small molecule inhibitor comprises a compound of Table 1, Table 2, or Table 3, or a pharmaceutically acceptable salt thereof.

49. The method of any one of claims 3-6, 13,23-27 and 34-38, further comprising administering to the subject a pharmaceutical composition comprising a synthetic activating transcription factor inhibitor (ATFi) and a suitable delivery vehicle, wherein the ATFi is a small-molecule inhibitor.

50. The method of claim 49, wherein the small molecule inhibitor is Ceapin A7, or a pharmaceutically acceptable salt thereof.

51. The method of claim 49, wherein the small molecule inhibitor comprises a compound of Table 1, Table 2, or Table 3, or a pharmaceutically acceptable salt thereof.

52. The method of claim 51, wherein the compound of Table 1, Table 2, or Table 3, or a pharmaceutically acceptable salt thereof, has at least one hydrogen atom with an isotopic distribution in proportional amounts different to those usually found in nature at any position that a hydrogen atom is present.

53. The method of claim 52, wherein the compound of Table 1, Table 2, or Table 3, or a pharmaceutically acceptable salt thereof, has one, two, three, four, five or six hydrogen atoms with deuterium in proportional amounts greater than usually found in nature.

54. The method of claim 53, wherein each of the one, two, three, four, five or six hydrogen atoms independently includes at least 52.5% deuterium incorporation, at least 60% deuterium incorporation, at least 67.5% deuterium incorporation, at least 75% deuterium incorporation, at least 82.5% deuterium incorporation, at least 90% deuterium incorporation, at least 95% deuterium incorporation, at least 97% deuterium incorporation, at least 99% deuterium incorporation, or at least 99.5% deuterium incorporation.

55. The method of claim 54, wherein each of the one, two, three, four, five or six hydrogen atoms includes less than 99.9% deuterium incorporation.
